(19) United States
(12) Patent Application Publication
(10) Pub. No.: US 2025/0118394 A1
(43) Pub. Date: Apr. 10, 2025

(54) AUTONOMOUS EXPLORATION

(71) Applicant: The University Court of the University of Glasgow, Glasgow (GB)

(72) Inventor: Leroy CRONIN, Glasgow (GB)

(21) Appl. No.: 18/727,969

(22) PCT Filed: Jan. 10, 2023
(86) PCT No.: PCT/EP2023/050487

§ 371 Date: Jul. 10, 2024

(30) Foreign Application Priority Data
Jan. 10, 2022 (GB) 2200261.2

(51) Int. Cl. G16C 20/10; C40B 40/00; C40B 50/00; C40B 60/02

(52) U.S. Cl. CPC G16C 20/10; C40B 40/00; C40B 50/00; C40B 60/02

(57) ABSTRACT

The invention provides a method for the exploration of chemical space through a multigenerational series of synthetic stages. In a first stage, a series of reactions is performed. The products of the first series are analysed, and a first product from the series of reactions is selected. The first product is used as a chemical input for each of the reactions in a second series of reactions. The products of the second series are analysed. The first and second stages are performed autonomously. The selection of a product in the first stage comprises the comparison of the products from the first series of reactions against a fitness function, where the selected product has a superior fitness compared with one or more other products in the first series. Each reaction in the first series differs in one or more chemical and/or physical inputs. Each reaction in the second series differs in one or more chemical and/or physical inputs. The invention also provides an automated exploration apparatus for performing the method, and the apparatus comprises a controller, and a chemical synthesiser and an analytical unit which are operable by the controller, as described herein.

### RELATED APPLICATION

The present case claims priority to, and the benefit of GB 2200261.2 filed on 10 Jan. 2022 (10.01.2022), the contents of which are hereby incorporated by reference in their entirety.

### FIELD OF THE INVENTION

The invention provides apparatus and methods for use in the autonomous exploration of chemical space, such as for use in the discovery and optimisation of nanomaterials.

### BACKGROUND

Nanomaterials possess unique size- and shape-controlled physical and chemical properties with applications in areas of medicine¹, electronics², catalysis³ and quantum technologies⁴. Controlling the morphology of nanomaterials is crucial for tuning their unique characteristics such as optical⁵, electrical⁶ and magnetic⁷ properties, but the synthesis often suffers from irreproducibility⁸, low yield⁹, and polydispersity¹⁰. Various bottom-up fabrication methods including electrochemical¹¹, photochemical¹², bio-templated¹³ and seed-mediated¹⁴ synthesis have been developed to create nanomaterials with desired properties. Despite the availability of various synthetic routes, finding optimal conditions for a target nanostructure with high shape yield and monodispersity is a huge challenge. This is due to the high dimensionality and sensitivity to the synthetic conditions such as reagent concentrations¹⁵, order of reagent addition¹⁶, temperature¹⁷ and mixing rate¹⁸. Despite this sensitivity, a standard, robust and unique digital signature that originates from both the synthetic procedure and validation of the synthesis is still lacking. These problems become more pronounced when multistep synthesis¹⁹ is required to achieve the targeted nanostructure.

The development of autonomous precision robotic architectures capable of parallel experiments in a closed-loop approach guided by machine learning (ML) algorithms can provide a viable path to address high dimensionality and sensitivity to synthetic conditions. Recently, various autonomous platforms have been developed for chemical synthesis^{20,21}, product separation^{22,23}, and in-line characterisation²⁴. It has also been demonstrated to accelerate material discovery by combining the autonomous platforms with customized ML algorithms²³⁻³⁰. However, recent automation of nanomaterials synthesis³¹ focuses on user-defined target-specific optimisation^{30,32} without exploration, and can still require manual steps³³. A system that unbiasedly performs open-ended exploration and searches a diversified set of high-performance products is still lacking. Quality Diversity (QD) algorithms³⁴ like Novelty Search with Local Competition³⁵ or Multi-dimensional Archive of Phenotypic Elites (MAP-Elites)³⁶ have been applied in several areas including real-time decision making³⁷, adaptive robotic control³⁸, de novo drug molecule discovery³⁹, and novel protocell behaviour search⁴⁰. In contrast to the classic optimisation algorithms that target a single highest-performance solution, QD algorithms can find solutions with both diversified behaviour and high performance, thus suitable to explore the chemical space and facilitate the diversity of products.

A crucial requirement for a closed-loop autonomous system is the selection of appropriate characterisation techniques³¹. Various characterisation techniques such as atomic force microscopy⁴¹, scanning electron microscopy⁴², transmission electron microscopy⁴³, dynamic light scattering⁴⁴ and small-angle X-ray scattering⁴⁵ are widely applied to investigate the morphology of nanomaterials. Although electron microscopy can provide detailed information on nanostructures, it is still impractical to implement it in the closed loop because of its cost and complexity. Considering the strong dependence of electromagnetic properties of metallic nanoparticles on the morphology and composition, in-line optical spectroscopy such as UV-Vis and infrared (IR) are optimal and practical characterisation techniques, and can be used as structural indicators. For open-ended exploration, increasing the diversity of spectral patterns could lead to the discovery of nanomaterials with distinct morphologies. The spectroscopic features such as peak prominence and broadness can be further utilised to search for synthetic conditions with higher yield and better monodispersity.

The present invention has been devised in the light of the above considerations.

### SUMMARY OF THE INVENTION

In a general aspect the invention provides apparatus and methods for use in the autonomous exploration of chemical space, such as for use in the discovery and optimisation of nanomaterials, as well as crystal materials and new compounds.

Accordingly, in a first aspect of the invention there is provided a method for the exploration of chemical space, the method comprising a multigenerational series of synthetic stages, such that the method comprises:
(i) a first stage, where a first series of reactions is performed, and an analysis of the reaction product for each reaction in the first series is performed, and a first product from the first series of reactions is selected; and
(ii) a second stage, where a second series of reactions is performed, where the selected product from the first stage is provided as a chemical input to each of the reactions in the second series, and an analysis of the reaction product for each reaction in the second series is performed, and a product from the second series of reactions is optionally selected,
wherein the first and second stages are performed autonomously, and the selection of a product in the first stage comprises the comparison of the products from the first series of reactions against a fitness function, where the selected product has a superior fitness compared with one or more other products in the first series, and
each reaction in the first series differs in one or more chemical and/or physical inputs, and each reaction in the second series differs in one or more chemical and/or physical inputs.

A selected product may be referred to as a seed for use in a next generation series of reactions. The selected product may be a template for the subsequent synthesis steps.

The methods of the invention are particularly suited to the preparation of nanomaterial products, such as nanoparticles. The methods of the invention may also be used more generally to prepare other materials, and may be used in the preparation of new compounds.

In the first stage more than one product may be selected, and each of the selected products has a superior fitness compared with one or more other products in the first series. Here, one of the selected product is used in a subsequent series of reactions, and each of the other selected products is used in separate and subsequent series.

The first and second stage are performed autonomously, for example using a robotic chemical synthesiser combined with a suitable controller and analytical unit, such as an automated exploration apparatus of the second aspect of the invention. The performance of the first and second stages autonomously includes the autonomous performance of the reactions using chemical and physical inputs supplied autonomously, the subsequent isolation of products autonomously, the selection of one or more products autonomously, and the supply of a selected product to a subsequent stage as a common reagent in a series of reactions. Thus, no user interference is needed other than the provision of the inputs to the chemical synthesiser and providing the appropriate controller, programmed to search for the products having the requisite fitness functions, and providing appropriate analytical units.

Thus, in one embodiment:
(i) in the first stage, a first series of reactions is performed, and an analysis of the reaction product for each reaction in the first series is performed, and a first and second product from the first series of reactions is selected; and
(ii) in the second stage, a second series of reactions is performed, where the first selected product from the first stage is provided as a chemical input to each of the reactions in the second series, and an analysis of the reaction product for each reaction in the second series is performed, and a product from the second series of reactions is optionally selected, and
the method further comprising:
(iii) a third stage, where a third series of reactions is performed, where the second selected product from the first stage is provided as a chemical input to each of the reactions in the third series, and an analysis of the reaction product for each reaction in the third series is performed, and a product from the third series of reactions is optionally selected,
wherein the first, second and third stages are performed autonomously, and the selection of the first and second products in the first stage comprises the comparison of the products from the first series of reactions against a fitness function, where the selected products have a superior fitness compared with one or more other products in the first series, and
each reaction in the first series differs in one or more chemical and/or physical inputs, each reaction in the second series differs in one or more chemical and/or physical inputs, and each reaction in the third series differs in one or more chemical and/or physical inputs.

The methods of the invention may include additional analytical steps to assess structural elements of the products, and to identify significant structural and chemical differences between reaction products in a reaction series.

In the methods of the invention, the selection of first and second products from the first reaction stage may comprise selecting first and second products having structural or chemical dissimilarity.

In the present method, the purpose of the first, second and third steps together is to provide for an extensive exploration of the chemical space. Subsequent steps, where present, may also be intended to provide for an extensive exploration of the chemical space. The selection of a plurality of products for use in a subsequent series of reactions may allow for the most rapid expansion and exploration of the available chemical space.

Following an initial expansion and exploration of an available chemical space it is expected that various lead products will be identified. These lead products are products having the most promising characteristics, scoring highly against the fitness function. These leads are then selected for optimisation. Here, there is no need for the selection of multiple products to expand into the chemical space. Rather a product may be selected for exploration of the space immediately around it.

Thus, whilst the initial exploration stage provides for expansion, the optimisation stages provide for fine control.

The method of the first aspect of the invention may comprise one or more additional optimisation stages, performed after the second stage, and optionally the third stage. The method further comprises:
(iv) an additional optimisation stage, where a series of reactions is performed, where the selected product from an earlier stage, such as a selected product from the second stage, or a selected product from the third stage, where present, is provided as a chemical input to each of the reactions in the additional series, and an analysis of the reaction product for each reaction in the additional series is performed, and a product from the additional series of reactions is optionally selected,
wherein the additional stage is performed autonomously, and the selection of a product in the additional stage comprises the comparison of the products from the additional series of reactions against a fitness function, where a selected product has a superior fitness compared with one or more other products in the first series, and
each reaction in the additional series differs in one or more chemical and/or physical inputs.

The method of the invention may comprise two or more, such as three or more, additional optimisation stages in generation, wherein each subsequent additional optimisation stage uses the selected product from the previous optimisation stage. Here, the method may be used to provide products that are inherited from two or more generations previous.

In an embodiment of the invention, a single product is selected from the series of products, and this product may be an elite product. An elite product may be a product having the highest fitness function amongst the series of products.

In a further embodiment, two or more, such as two, products are selected from the series of products. One of those products may be an elite product. Any other products selected may be those products having the highest fitness functions after the elite product.

In one embodiment, the fitness function of a product is determined by the optical, such as spectroscopic properties, of the product.

The spectroscopic properties of the products may be UV-vis or IR spectroscopic properties. The spectroscopic properties may include absorption properties, such as absorption minima and maxima.

The fitness function may also be determined with reference to the mass-spectral properties of a product, as determined by mass spectroscopy, the retention time of a product as determined by a chromatographic technique, such as HPLC, or the nuclear magnetic resonance properties of a product as determined by as determined by NMR spectroscopy.

The method of the invention operates autonomously, with a suitably programmed controller operating a synthesiser in conjunction with an analytical unit. Thus, the method steps of the invention require no user input.

It is preferred that a product is, or is selected as, a solid product. This solid product may be a crystal. A product may be a particle, and is preferably a nanoparticle or nanorod. In an alternative embodiment, the product may be a liquid, such as liquid product that is immiscible with the other components of the reaction mixture.

The methods of the invention include those where the method proceeds continuously within each stage. Thus, the method is performed without halt until at least the final product in the series is prepared. This has the benefit of reducing downtime in the methods of the invention.

The methods of the invention include those where the method proceeds continuously from one stage to a following stage. Thus, the method is performed without a halt between the stages. This has the benefit of reducing downtime in the methods of the invention.

The methods of the invention include those where a part of a later series of reactions may be performed coincident with the performance of a part of an earlier series of reactions. Thus, a subsequent series of reactions may be initiated for a later stage whilst the method is still working to finish an earlier series of reactions within an earlier stage. In this situation a first product may be selected from the earlier series of reactions before that series of reactions is complete, and that selected first product may then be used as a chemical input in a subsequent series of reactions. Here, during the performance of a first series of reactions, a first product having an excellent fitness may be identified early in the series of reactions, and that first product may then be selected for use in subsequent stages. The earlier series of reaction is nevertheless completed, as further products having desirable fitness functions may still be produced, and they may be selected for use in alternative subsequent stages.

In a second aspect the present invention provides an automated exploration apparatus for performing a method for the exploration of chemical space, and the apparatus comprises a controller, and a chemical synthesiser and an analytical unit which are operable by the controller, wherein:
(i) the chemical synthesiser having a plurality of reaction spaces; a supply of chemical inputs, optionally together with physical inputs for supplying to the chemical inputs, optionally together with physical inputs to a reaction space; a product separator for at least partial separation of a reaction product from a reaction product mixture in a reaction space, and for supply of the reaction product to a separate reaction space;
(ii) the analytical unit for analysing a product of a reaction performed in a reaction space of a chemical synthesiser; and
(iii) the controller is suitably programmed to operate the chemical synthesiser and the analytical unit; to receive analytical information from the analytical unit; and to compare the analytical information for reaction products against a fitness function; to make a selection of a reaction product for the product separator to supply to a separate reaction space; and to make a selection of chemical inputs, optionally together with physical inputs, to the reaction spaces
and the controller is adapted to operate the chemical synthesiser and the analytical unit autonomously.

In one embodiment the plurality of reaction spaces is the plurality of reaction spaces with a Geneva wheel.

The analytical unit preferably includes a spectrometer, such as a UV-vis spectrometer or an IR spectrometer.

The analytical unit may analyse a product within a reaction product mixture, or it may analyse a product following its at least partial separation from the reaction product mixture. Here, the analytical unit cooperates with the product separator to make the product available for analysis.

The product separator is for collecting a product from a reaction mixture. The collection of the product may include the at least partial purification of the product from other components of the reaction product mixture, such as one or more of a solvent, unreacted reagents, catalysts, and by-products. The product may be collected from a work-up of the reaction mixture. The work-up may include filtration phase separation, concentration and drying amongst others.

The product separator may make available a product for the analytical unit to analyse.

The product separator is under instruction from the controller. Under such instruction, the product separator may provide a product, which may be a selected product, to a reaction space for use as a reagent in a series of reactions. Thus, the product separator delivers the seed for a subsequent generation of reactions.

In one embodiment the product separator is provided with a storage unit for storing a reaction product. A reaction product is deliverable to the storage unit, for example after at least partial purification from a reaction product mixture, and is then made available from the storage unit to a reaction space as needed.

The product separator is capable of supplying a reaction product to multiple reaction spaces, such as multiple reaction spaces in series.

Where needed, a product may be formulated for appropriate delivery to a reaction space. Here, the controller may operate the chemical and physical inputs to the reaction spaces in combination with the reaction product, to give the appropriate formulation of the reaction product for the reaction space.

The automated exploration apparatus may be suitable for use with the method of the first aspect of the invention.

Within the automated exploration apparatus, the controller is suitable programmed with an AI algorithm to analyse analytical data and to select products for use in a series of reactions, and to select future chemical inputs, optionally together with physical inputs, for use with the selected product a series of reactions.

The invention also provides a library, which library is a collection of products, such as nanomaterial products, produced by the method of the first aspect of the invention. A library may be provided with an electronic instruction set for one or more, such as each, product, which instruction set is an experimental description of a method for obtaining the nanomaterial product using an automated chemical synthesiser.

A library may be a collection of the selected products obtained or obtainable from each stage in the methods of the invention.

These and other aspects and the embodiments of the invention are described in further detail herein.

### SUMMARY OF THE FIGURES

The present invention is described with reference to the figures listed below.

FIG. 1—The closed-loop approach towards exploration and optimisation in the seed-mediated synthesis of nanoparticles. (a) A pictorial representation of Au NPs from hierarchically-linked chemical synthetic spaces in the seed-mediated synthesis. (b) The closed-loop approach for exploration ((i), blue cycle) and optimisation ((ii), red cycle), respectively. In the exploration, the UV-Vis features of samples are extracted to evaluate their behaviour and performance respectively. New experiments are designed to increase the spectral diversity and performance of samples. After exploration, transmission electron microscopy (TEM) is used to reveal the morphologies of the high-performance samples, which offers target spectrum for optimisation through a scattering simulation engine. In the optimisation, the UV-Vis spectra of samples are compared to a target spectrum and new experiments are designed to search multiple nanostructures with high similarity.

FIG. 2—The autonomous nanomaterials discovery platform. (a) The workflow of the closed loop including synthesis, analysis, and design of new experiments by the algorithms. (b)-(c) The CAD design and the experimental set-up of the chemical reaction module driven by the Geneva wheel with units for liquid dispensing, pH control and solution transfer. (d) The overall set-up of the autonomous platform with temperature controller, stock solutions, pumps, chemical reaction module, flow cells, light sources, and spectrometers.

FIG. 3—Exploration and optimisation in the simulated chemical space. (a) The simulated chemical space with various shapes, Au/Ag compositions and shape yields, with their corresponding simulated UV-Vis spectra through discrete-dipole-approximation. (b) The investigation of the chemical space with the exploration algorithm from AI-EDISON. The class index (from 1 to 10 with an additional class of 0 for non-featured samples) is assigned depending on the peak position. The fitness values of the samples were evaluated to update the parent set iteratively. (c) The phase volumes and interconnectivities of different classes in the simulated space. (d) The comparison of the performance in both discovering samples of new classes (top) and increasing the fitness of the highest-performance samples within various classes (bottom) between the exploration algorithm from AI-EDISON and Random Search with 16 repeats. After 200 steps, Random Search still cannot find samples of all the classes with a standard deviation of 0.5 among the repeats. The exploration algorithm outperforms the final result from Random Search after 27 steps and can find samples belonging to all classes after 78 steps. (e) The investigation of the chemical space with the optimisation algorithm from AI-EDISON after exploration. (f) The UV-Vis spectra of solutions with the highest similarity after the exploration of 11,21,31 and 41 steps, indicating the unsuccessful search towards a target purely by exploration. (g) The increase of the similarity metric during the optimisation with varying importance of the local sparseness, along with exploration strategy. The linear coefficient (k) is changed from 0 to 300. (h) An example of the UV-Vis spectra and their corresponding nanostructures of three solutions after optimisation. Both the global maximum and local maxima were found.

FIG. 4—The exploration to discover uniquely-shaped Au NPs in three linked chemical spaces. (a) The electron micrographs of the obtained Au NPs and their synthetic trajectories in the seed-mediated synthesis. L1-5 and L2-12-2 were used as the seeds after exploration. The three chemical spaces are indicated by the red, blue, and green background. The scale bars are 50 nm (blue) and 100 nm (red) respectively. (b) The UV-Vis spectra corresponding to the Au NPs from three chemical spaces. It should be noted L2-11-1, L2-11-2 and L2-12-2 showed very similar UV-Vis spectra. The grey dotted lines indicate the discrete subregions to facilitate the UV-Vis diversity during exploration.

FIG. 5—The optimisation towards target spectra from the scattering simulation of Au NPs. (a) The comparison of the UV-Vis spectra from the simulation of target Au nanorods, the most similar sample before optimisation, and the optimal solution after optimisation. (b)-(c) The electron micrographs of the best sample before optimisation with a yield ca. 57%. and the optimal solution after optimisation with a yield ca. 95%. (d) The target UV-Vis spectrum derived from Au octahedra. (e) The comparison of the UV-Vis spectra of the simulated Au octahedral target and three solutions after optimisation. (f) The comparison of the synthetic conditions of the three solutions after the optimisation (AA: ascorbic acid). (g)-(i) The electron micrographs of solution 1-3, which corresponds to octahedral, concave octahedral, smooth polyhedral nanoparticles. The scale bars are 100 nm.

FIG. 6—The fully autonomous multistep synthesis with directed graph structure. (a) The workflow of the autonomous multistep synthesis of Au NPs. A graph structure is used to allocate the available hardware resources to samples on the chemical reaction module and design the operations to be executed for the synthesis. (b) The six target Au NPs, their hierarchical relations and distribution on the chemical reaction module. They are labelled from N1-N6, which corresponding to L1-5, L1-1, L2-12-2, L2-7, L3-3, L3-1 respectively. (c) The synthesis, reaction, and hardware graph for the multistep synthesis of the six Au NPs. (d) The UV-Vis spectra of samples from three repeats and the original sample. A means wavelength. The most prominent peak position is 799.6±6.5 nm, 525.9±0.9 nm, 530.0±1.2 nm, 777.0±6.9 nm, 673.4±2.6 nm, and 561.0±3.4 nm from N1 to N6 respectively. (e) The actual distribution of the samples on the chemical reaction module. (f) The procedure to generate the unique digital signatures of nanoparticles through χDL and product validation in the multistep synthesis.

FIG. 7—Flow diagram of the operations of the platform in the CRM. (a) Initialisation of the hardware loads the platform configuration (devices connected and their attributes), homes moving components with positioning sensors, ensures access to spectrometers and aligns the pump valves to their default positions. (b) Pumps are primed with reagents to eliminate dead volume dispensing from the start. (c) A water UV-Vis reference is obtained for later analysis and removed. (d) Reactions are performed in which: (d)[i] reagents are dispensed, stirred and reductant added, (d)[ii] pH is measured/controlled, and metallic salt as well as premade gold seeds are dispensed and (d)[iii] in parallel, the pH probe is moved to and cleaned at its wash once the pH control is finished. (e) After a growth period, (e)[i] the samples are transferred to the flow cells and analysed. After analysis (e)[ii] the sample is moved to waste and flow apparatus is cleaned to get ready for the next sample. (f) Data is fed into an algorithm to produce the next series of reactions. (g) During a multistep synthesis procedure, certain samples can be moved to other samples as the seeds or stored temporarily using the seed transfer unit. The synthetic procedure of adding reagents in (d) can vary during exploring different chemical spaces and will be discussed respectively.

FIG. 8—The scheme for exploration algorithm based on MAP-Elites. All the samples in the chemical space are projected to the feature space according to their fitness and attribute. Depending on the attribute, certain criteria was used classify samples with different behaviours. Here we discretize the behaviour space into multiple subregions, and the classificatoin was conducted by selecting which subregion the sample are located in. The sample with the highest fitness within each class is regarded as an elite and added to the parent set. New experiments are generated by the crossover and mutation of the parents, and the chemical space was explored by iterating this process.

FIG. 9—A typical procedure to simulate the optical properties of nanoparticles. The original atomic model or the continuum geometry is approximated by a set of dipoles and after solving the dipoles, the spectroscopic properties and local electric field distribution are further evaluated. Here we used Au octahedron (edge length=20 nm) as an example with its simulated spectrum. Its local electric field distribution at 560 nm is shown. For local electric field simulation, the incident direction is from +Z to −Z and the polarization direction is from −X to +X. Continuum geometry was used to generate the dipoles with a length of 1.0 nm.

FIG. 10—The comparison between DDSCAT and PyDScat-GPU. The extinction efficiency factors were calculated with these two methods (DDSCAT: black line; PyDScat-GPU: blue line) for different Au nanostructures including spheres (a), octahedra (b), transverse (c) and longitudinal (d) mode of rods. The raw data points are shown in dots with a range of [0.4, 0.8] μm with an interval of 0.01 μm and cubic spline interpolation was applied to smooth the curve. All the dipole sets were generated from continuum geometries.

FIG. 11—The simulated UV-Vis spectra of bimetallic Au-Ag nanoparticles. (a) The Ag portion of the Au-Ag octahedron (edge length≈20 nm) is increased from 0% to 50%, with a blue-shifted peak in the simulated spectra resulting from the polarizability change. (b) The Au core size is decreased from 20 nm to 10 nm as the thickness of Ag shell increases. The original peak corresponding to the Au feature disappears, and new Ag feature peaks appear during this process.

FIG. 12—Examples of the shapes originated from superellipsoid by tuning the parameter set (a,b,c,r,t). The corresponding parameters are labelled with the individual shape, together with the dipoles (yellow dots) and contour (red) in this figure. c was used to elongate the shape and (r,t) can change the curvature gradually from smooth to sharp. For every shape, a set of dipoles was created for further DDA simulation.

FIG. 13—Examples of the shapes of the set of Au nanoparticles and the corresponding UV-Vis spectra in simulated chemical space 1. The dipoles are purely composed of Au. The corresponding parameter set (a,b,c,r,t) for the shape is labelled in the figure. The dipole size was selected and enabled to satisfy Eq. (14) and multiple orientations were calculated and averaged for the final UV-Vis spectrum. The wavelength range is [0.4, 0.9] μm with an interval of 0.01 μm and cubic spline interpolation was applied to smooth the curve. The extinction was normalized to the range of [0,1].

FIG. 14—The portion of Ag (p_{Ag}) as a function of the relative distance (d_{R}). Different curves were drawn by setting v_{DCF,1} as 50 and changing v_{DFC,2} from 0.9 to 0.6 with an interval of 0.1. With the decreased value of v_{DCF,2}, the portions of Ag in the outside dipoles are increased.

FIG. 15—Examples of the shapes of the set of Au—Ag nanoparticles and the corresponding UV-Vis spectra in simulated chemical space 2. The dipoles are composed of Au and Ag with their portions in the dipole indicated by the colour. The corresponding parameter set (a, b, c, r, t, v_{DCF,2}) for the shape is labelled in the figure. The dipole size was selected to satisfy Eq. (14) and several orientations were calculated and averaged for the final UV-Vis spectrum. The sampling range is [0.4, 0.9] μm with an interval of 0.01 μm and cubic spline interpolation was applied to smooth the curve. The extinction was normalized to the range of [0,1].

FIG. 16—The analysis of the simulated chemical space 1. (a) The pictorial representation of the class and their interconnectivity in simulated chemical space 1. The sizes of the nodes indicate the phase volumes of the classes, and the thickness of the edges indicate the interconnectivity. Class 0 indicates the samples with peak number smaller than 2. (b) The estimated percentages of the volumes in the input space for different classes. They are 41.04%, 0%, 0%, 3.26%, 9.78%, 11.21%, 12.77%, 12.31%, 7.50%, 1.93% and 0.20% from class 0 to class 10 respectively. Note no sample belonging to class 1 and 2 in the first chemical space, indicating no double-peak system can have the most prominent peak in the range of [0.4, 0.5] μm.

FIG. 17—The analysis of the simulated chemical space 2. (a) The pictorial representation of the class and their interconnectivity in simulated chemical space 2. The sizes of the nodes indicate the phase volumes of the classes, and the thickness of the edges indicate the interconnectivity. Class 0 indicates the samples with peak number smaller than 2. (b) The estimated percentages of the volumes in the input space for different classes. They are 36.18%, 19.42%, 0.28%, 13.49%, 9.68%, 11.55%, 7.12%, 1.66%, 0.56%, 0.04% and 0.01% from class 0 to class 10 respectively.

FIG. 18—In silico exploration of nanoparticles with exploration algorithm based on MAP-Elites in simulated chemical space 1. (a) A simulated chemical space was based on superellipsoid shape descriptor and simulated extinction spectra. (b) The scheme for exploration. The results were characterised and classified according to their UV-Vis behaviour, and only the elites were selected as parents to generate the next set of samples. (c) The results of exploration with both exploration algorithm based on MAP-Elites (red) and Random Search (blue) with the estimated upper boundary (black) in the simulated chemical space. The performance of the exploration algorithm after 15 (to find elites) or 8 (to increase fitness) steps is better than the final results of Random Search after 51 steps.

FIG. 19—In silico optimisation in simulated chemical space 2. (a) The UV-Vis spectra with the highest similarity to the target after running exploration for 11, 21, 31 and 41 steps, as well as the target spectrum itself. (b) The increase of the similarity with varied coefficients (k₃ in Eq. (3)) of local sparseness and different initial data set. The step where we started the optimisation process was labelled as red in the figure. The base of the logarithmic scale is 10. Note the results from optimisation were averaged among 16 parallel repeats. The results from the exploration algorithm based on MAP-Elites are indicated by the grey line.

FIG. 20—The difference in the input space defined by (v₁, v₂, v₃, v₄, v₅) between the best solution and the target. The optimisation algorithm with varied k₃ was initialised after running exploration for 11 (a), 21 (b), 31 (c) and 41 (d) steps. The differences were calculated as discussed above and averaged among 16 parallel repeats. The differences from the exploration algorithm based on MAP-Elites are indicated by the grey line.

FIG. 21—The distribution of the similarity metrics of the local maxima, and their distance to the closest solutions from optimisation. (a) The similarity metrics of the local maxima. They are sorted in the descending order regarding their similarity metrics. (b) The smallest distance between the solutions and the local maxima calculated in the parameter space of (c, r, t, v_{DCF,2}, v₅). In calculating the distance, every dimension was normalized from 0 to 1 by a liner transformation.

FIG. 22—The distributions of the similarity metrics on the planes passing through the local maxima in the nanostructure parameter space of (c, r, t, v_{DCF,2}, v₅). The red points indicate the local maxima, and the cross indicate the projection of the nearest solution. The colour bar was used to show the similarity distribution on the two-dimensional plane. The similarity metrics were normalized to the range from 0 to 1 by a linear transformation.

FIG. 23—The five UV-Vis spectra (a) and the corresponding nanostructures (b) from the top five solution from optimisation with starting step of 11 and k₃=100. Solution 1 is the global maximum.

FIG. 24—The unique local maximum number N_{L} (a) and the least sampling number Nt (b) before (red) and after (blue) ten-nearest neighbour selection.

FIG. 25—The scheme of the multistep synthesis strategy. Starting with an initial seed, the chemical space was explored and different nanostructures were discovered. The chemical space was further expanded using the found nanostructures as new seeds to create a diversified map of nanostructures.

FIG. 26—The flow diagram of the exploration strategy with the autonomous platform.

FIG. 27—The scheme of the exploration strategy with two different scenarios in the multiple peak system. Two different fitness functions (F₂, F₃) were used to facilitate the diversity of UV-Vis features, thus resulting into distinct elites belonging to two different classes.

FIG. 28—The demonstration of the stability of the platform. (a) The normalized UV-Vis spectra of the standard samples from different steps to demonstrate the stability of the autonomous platform during the exploration. (b) The different peak positions corresponding to the longitudinal mode of nanorods. The mean value is 793 nm with a standard deviation of 5 nm. All peak positions are within 3 standard deviations of their mean value as indicated by the red background.

FIG. 29—The scheme of the newly added operations in exploration algorithm in a two-dimensional space. (a) The mutations based on original sampling points in different directions without hitting any boundaries. (b) The mutation from an original sampling point that hits the boundary. The perturbation vector is thus scaled down. (c) Possible valid (black arrows) and invalid (red arrows) mutations of a sampling point on the boundary. (d) The crossover of two samples resulting in an offspring out of the boundary. The variables of the offspring are scaled down to the boundary. All the valid/invalid perturbations are represented by black/red arrows.

FIG. 30—The open-ended exploration of the first chemical space with multiple systems. (a) The number of elites found during open-ended exploration. S (blue line), M1 and M2 were used to indicate the single and multiple-peak systems with the different scenarios respectively. (b) The distribution of fitness values of different elites throughout the exploration. Depending on the class of the elite, the fitness value can vary and several samples show a large increase during the exploration. (c) The final UV-Vis spectra of the elites in the single-peak system after exploration. (d) The final UV-Vis of the elites in the multiple-peak system (scenario 1) after exploration. (e) The final UV-Vis of the elites in the multiple-peak system (scenario 2) after exploration. (f) The elite distribution in the 3-dimensional space defined by fitness value, elite class and steps. (g) The evolution of elites via crossover, mutation and random sampling in the exploration. The red and blue arrows indicate the propagation of experimental conditions to a new elite through mutation and crossover. The three regions correspond to three sets: S, M1 and M2. Note when the initial parent number is small, one elite can be sampled repeatedly in the crossover, but only its experimental conditions are used to generate the new experiment. Elite-C, Elite-M and Elite-R in (f) and (g) represent the new elites from crossover, mutation and random sampling respectively.

FIG. 31—The comparison of the UV-Vis spectra of four Au nanorods with improved absorption band before (red) and after (blue) the two stages. (a) to (d) corresponds the best spectrum in the same class as R1 to R4 respectively. The comparison of the same class as R5 is not shown because the absorption band was not increased in the later 6 steps.

FIG. 32—The increase of the highest absorption band defined in Eq. (25) during different stages with 10, 4 and 2 steps respectively. (1)-(3) represents the open-ended exploration, constrained exploration and exploitation. (a) to (d) corresponds the increase of the fitness within the same class as R1 to R4 respectively.

FIG. 33—The UV-Vis spectra of the five Au nanorods labelled with R1 to R5. The aspect ratio is increased among them as indicated by the red-shifted peak positions that correspond to the longitudinal mode.

FIG. 34—The pH control in chemical space 2. (a)-(b) Two examples of the pH control. The targets pHs are 7.80 and 6.00 respectively. The final pHs are 7.72 and 5.98 respectively. (c) The target pHs (red) and the corresponding final pHs (blue) from samples during exploring chemical space 2. The target pHs were sorted in ascending order. (d) The distribution of the absolute difference between target pH and final pH (|ΔpH|). 443 (96%) samples have the final pH within 0.2 range of the target pH. The maximum difference between the target pH and the final pH is 0.32. Considering the difference, the final pH was transformed to the algorithm variable to design new experiments.

FIG. 35—The demonstration of the stability of the platform. (a) The normalized UV-Vis spectra of the standard samples during exploration in different batches. (b) The highest peak positions from the standards. The mean value is 681 nm with a standard deviation of 5.6 nm. All peak positions are within 3 standard deviations of their mean value as indicated by the red background.

FIG. 36—The exploration of chemical space 2. (a) An example of controlling pH via the proportional algorithm to reach the target pH. (b) The number of elites found via different steps in the 10 steps only considering the multiple-peak systems in stage 1. (c) The number of elites found via different steps in the 10 steps only considering the single-peak system in stage 2. S, M1 and M2 were used to indicate the single-peak system and the multiple-peak systems of different scenarios including making a single peak dominant or two peaks comparable respectively. Note the multiple-peak systems of different scenarios have the same number of elites. (d) The final UV-Vis of the elites in the multiple-peak system scenario 1 after exploration. (e) The final UV-Vis of the elites in the multiple-peak system scenario 2 after exploration. (f) The final UV-Vis of the elites in the single-peak system after exploration. The boundaries to define the subregions for different classes were drawn by dotted lines in (d) to (f).

FIG. 37—The evolution of the elites from different classes during exploring chemical space 2. In stage 1 (a), Elite, Elite-C, Elite-M and Elite-R correspond to elites without any change, elites from the crossover, elites from the mutation and elites from random sampling from the last step respectively. In stage 2 (b), Elite-I indicates the elites from the initial data set of the multiple-peak system exploration and there is no random sampling contributing to the exploration. In the multiple-peak system exploration, Elite 1 to 17 belong to the scenario 1 and 18-34 to scenario 2.

FIG. 38—The demonstration of the stability of the platform. (a) The normalized UV-Vis spectra of the standard samples during exploration in different steps (including the original spectrum in the first step). (b) The different peak positions of the standard samples. The mean value is 673 nm with a standard deviation of 6.2 nm. All peak positions are within 3 standard deviations of their mean value as indicated by the red background.

FIG. 39—The exploration of the third chemical space based on Au nanospheres. (a) The number of elites found via different steps in the 10 steps. (b) The final UV-Vis of the elites with the boundaries of subregions to distinguish classes. (c) The evolution of elites via crossover, mutation and random sampling in the exploration. The red and blue arrows indicate the propagation of experimental conditions to a new elite through mutation and crossover. In (c), Elite, Elite-C, Elite-M and Elite-R correspond to elites without any change, elites from the crossover, elites from the mutation and elites from random sampling from the last step respectively.

FIG. 40—The time cost distribution during exploring chemical space 2 with pH control. The time cost for solution preparation (liquid dispensing and pH control), waiting for growth and UV-Vis characterisation and cleaning are shown in different colours.

FIG. 41—The time cost distribution during exploring chemical space 3 with only liquid dispensing. The time cost for solution preparation (liquid dispensing and pH control), waiting for growth and UV-Vis characterisation and cleaning are shown in different colours.

FIG. 42—The flow diagram of the optimisation strategy with the autonomous platform using optimisation algorithm based on GS-LS.

FIG. 43—The demonstration of the stability of the platform. (a) The normalized UV-Vis spectra of the standard samples in different batches to measure the stability of the autonomous platform during the optimisation. (b) The different peak positions corresponding to the longitudinal mode of nanorods. The mean value is 796 nm with a standard deviation of 2 nm. All peak positions are within 3 standard deviations of their mean value as indicated by the red background.

FIG. 44—The UV-Vis of the target from simulation and solutions in optimisation. The similarity metric of solution 1 to 2 is −54.85 and −53.09 respectively while before optimisation, the highest similarity is −57.22.

FIG. 45—The synthetic condition distribution of the top two solutions from the optimisation. The solutions are labelled by the cross and their neighbours are labelled by the red points. The similarity metric distribution of the rest of the samples are shown in the same figure. The colour bars show the similarity metrics of samples and are in range from −400 to −50. AA: ascorbic acid.

FIG. 46—The demonstration of the stability of the platform. (a) The normalized UV-Vis spectra of the standard samples in different batches to measure the stability of the autonomous platform during the optimisation. (b) The different peak positions from the standard samples. The mean value is 612 nm with a standard deviation of 2 nm. All peak positions are within 3 standard deviations of their mean value as indicated by the red background.

FIG. 47—The UV-Vis of the target from simulation and solutions in optimisation. The similarity metric of solution 1 to 5 is −12.17, −10.97, −10.14, −10.07 and −13.92 respectively.

FIG. 48—The synthetic condition distribution of the solution 1 from the optimisation. The solution is labelled by the cross and its neighbours are labelled by the red points. The similarity metric distribution of the rest of the samples is shown in the same figure. The colour bars show the similarity metrics of samples and are in range from −260 to −10. AA: ascorbic acid.

FIG. 49—The synthetic condition distribution of the solution 2 from the optimisation. The solution is labelled by the cross and its neighbours are labelled by the red points. The similarity metric distribution of the rest of the samples is shown in the same figure. The colour bars show the similarity metrics of samples and are in range from −260 to −10. AA: ascorbic acid.

FIG. 50—The synthetic condition distribution of the solution 3 from the optimisation. The solution is labelled by the cross and its neighbours are labelled by the red points. The similarity metric distribution of the rest of the samples is shown in the same figure. The colour bars show the similarity metrics of samples and are in range from −260 to −10. AA: ascorbic acid.

FIG. 51—The synthetic condition distribution of the solution 4 from the optimisation. The solution is labelled by the cross and its neighbours are labelled by the red points. The similarity metric distribution of the rest of the samples is shown in the same figure. The colour bars show the similarity metrics of samples and are in range from −260 to −10. AA: ascorbic acid.

FIG. 52—The synthetic condition distribution of the solution 5 from the optimisation. The solution is labelled by the cross and its neighbours are labelled by the red points. The similarity metric distribution of the rest of the samples is shown in the same figure. The colour bars show the similarity metrics of samples and are in range from −260 to −10. AA: ascorbic acid.

FIG. 53—Examples of the synthesis graph, reaction graph and the corresponding hardware graph. The synthesis graph is composed of multiple desired nanoparticles and their hierarchical relationships. The reaction graph designs the chemical reactions according to the synthesis graph. The hardware graph distributes the resources of the hardware for the parallel synthesis of multiple samples and defines the seed solution transfer among samples. The slots with grey or dark colour in the hardware graph indicate that they are used to flush the system or not used in the experiments, respectively. The initial seed was labelled as S.

FIG. 54—The synthesis graph, reaction graph and hardware graph for multistep synthesis of six nanoparticles. Every nanoparticle was repeated three times so there were 18 experiments in total. The sample that will be characterised with UV-Vis was labelled as blue. The initial seed was labelled as S.

### DETAILED DESCRIPTION OF THE INVENTION

The present invention provides apparatus and methods for use in the autonomous exploration of chemical space, such as for use in the discovery and optimisation of products, including nanomaterials.

The methods of the invention are mutigenerational exploration methods, a feature of which is that a selected product from one series of chemical reactions is used as common a starting material for reactions in a subsequent series of chemical reactions. A selected product from that subsequent series of reactions may then itself be used as a common starting material in a yet further series of chemical reactions. In this way, the physical or chemical attributes of a product may be carried through multiple series of reactions. Here, the methods of the invention may be regarded as providing multiple generations of products, with the products providing a hereditary chemical or physical element to be passed from one series to another. The multiple stages of the methods may be regarded as generational steps of the method.

U.S. Pat. No. 5,463,564 describes an iterative process for generating chemical entities. The process involves synthesising a library from building blocks contained in a reagent repository, analysing the structure-activity relationship of products in the library and ranking the products by their properties, then generating a new set of instructions for synthesising a new library with improved properties.

The process in U.S. Pat. No. 5,463,564 differs from the present invention in that an input for the chemical reactions at each stage of the process is not a product selected from a previous stage. Rather, the starting material is selected from the same pool of building blocks in each stage, and it is the combination of building blocks that is changed between stages (see, for example, column 4, paragraph 2 and column 6, paragraph 4).

In a preferred embodiment, the method of the invention specifies that both a second and third reaction stage are performed with each stage using a different product formed in the first stage synthesis. The selection of multiple products during an exploration is advantageous because the available chemical reaction space is further expanded in this way.

The apparatus of the invention comprises a controller programmed to select a reaction product for a product separator to supply to a separate reaction space, and such is not disclosed in the apparatus of U.S. Pat. No. 5,463,564.

WO 2009/053691 relates to a method of evolving and selecting nucleic acid aptamers. The method includes steps of binding nucleic acid sequences to a target, applying a fitness function to the bound sequences and selecting sequences with aptameric potential, then evolving the sequences to generate a new nucleic acid pool (see Abstract and pages 8 to 9). There is a general disclosure that the evolution step can include reproduction, recombination, cross-over and mutation of sequences, which is said to be performed between iterations of the selection process (at page 10, second paragraph).

Within the worked examples of WO 2009/053691 a method is described that involves an initial stage of selecting and analysing aptamer sequences, followed by synthesis of a new library of nucleic acids using a DNA synthesiser programmed to incorporate crossover combinations of motifs that are identified in the initial library, or to introduce random mutations at specific positions (see pages 25-29, in particular at page 28, paragraph 3). This differs from the present case, which requires the product of a previous stage to be used directly as the common input in a subsequent stage.

Furthermore, there is no reference in WO 2009/053691 to an exploration method that is an autonomous process.

The methods of the present case are suitable for identifying a product having desirable chemical and/or physical characteristics. The methods of the invention are intended to explore chemical space, to find one or more products having a desired or optimal chemical and/or physical characteristic.

The methods of the invention allow for the products in a series of reactions to be analysed, and the analytical information may be used to assess the properties of a product against a fitness function, which function is derived from the desirable chemical and/or physical characteristic. A product that is ranked highly against the fitness function may be selected for use in a subsequent series of reactions.

Where a product has the highest ranking against the fitness function compared against the other products in the series, that product may be referred to as the elite. The methods of the invention preferably look to identify these elites, and to use them as the starting materials, such as a common reagent, in subsequent series of reactions.

The methods of the invention are performed autonomously. Thus, the syntheses of products in a reaction series, the analysis of the reaction products, and their assessment against the fitness function may be controlled by a suitably programmed controller. This controller also selects the appropriate product for use as a common chemical input in a subsequent series of reactions.

It is the controller that also decides when to halt the method of the invention. The method may be halted when a product is obtained that has or exceeds the fitness values specified as the target value. The method may be halted where a maximum fitness value is obtained and additional stages in the method do not improve or substantially improve that fitness value, for example, after two or three additional stages.

A method may also be halted where there is a noticeable deterioration in fitness values after one, two or three or more additional stages.

However, it is not always necessary to halt the method where there is a deterioration of the fitness values, or there is no noticeable improvement in fitness values. In the alternative, the method may include the step or reverting to an alternative selected product from an earlier stage of the method, and using that alternative selected product as the basis for future generations. This alternatively selected product may provide access to alternative product structures through its use, and these alternative products may have altered, such as improved fitness values.

The methods of the invention may be used to explore chemical space, and they are not particularly limited to any one type of chemical structure, or any one type of chemical reactivity. By way of example, the present case demonstrates the preparation of products that are nanostructures, and more specifically nanorods.

Accordingly, the present invention provides a method for the exploration of chemical space. The method may be regarded as having a multigenerational series of synthetic stages. The method comprises:
(i) a first stage, where a first series of reactions is performed, and an analysis of the reaction product for each reaction in the first series is performed, and a first product from the first series of reactions is selected; and
(ii) a second stage, where a second series of reactions is performed, where the selected product from the first stage is provided as a chemical input to each of the reactions in the second series, and an analysis of the reaction product for each reaction in the second series is performed, and a product from the second series of reactions is optionally selected,
wherein the first and second stages are performed autonomously, and the selection of a product in the first stage comprises the comparison of the products from the first series of reactions against a fitness function, where the selected product has a superior fitness compared with one or more other products in the first series, and
each reaction in the first series differs in one or more chemical and/or physical inputs, and each reaction in the second series differs in one or more chemical and/or physical inputs.

A detailed discussion of the method steps is set out below.

The invention also provides an autonomous exploration apparatus for use in such methods. This apparatus may be referred to as a chemical robot.

The autonomous exploration apparatus comprises a controller, and a chemical synthesiser and an analytical unit which are operable by the controller, wherein:
(i) the chemical synthesiser having a plurality of reaction spaces; a supply of chemical inputs, optionally together with physical inputs for supplying to the chemical inputs, optionally together with physical inputs to a reaction space; a product separator for at least partial separation of a reaction product from a reaction product mixture in a reaction space, and for supply of the reaction product to a separate reaction space;
(ii) the analytical unit for analysing a product of a reaction performed in a reaction space of a chemical synthesiser; and
(iii) the controller is suitably programmed to operate the chemical synthesiser and the analytical unit; to receive analytical information from the analytical unit; and to compare the analytical information for reaction products against a fitness function; to make a selection of a reaction product for the product separator to supply to a separate reaction space; and to make a selection of chemical inputs, optionally together with physical inputs, to the reaction spaces
and the controller is adapted to operate the chemical synthesiser and the analytical unit autonomously.

A detailed discussion of the autonomous exploration apparatus is set out below.

It follows that the present invention provides a cyber-physical robot for the exploration, discovery, and optimisation of nanostructures which is driven by real-time spectroscopic feedback, theory and machine learning algorithms that control the reaction conditions and allow the selective templating of the reactions.

This approach allows the transfer of materials as seeds, as well as digital information, between cycles of exploration, opening the search space like gene transfer in biology.

By way of example, as shown in the Experimental Section of the present case, open-ended exploration of the seed-mediated multistep synthesis of gold nanoparticles (AuNPs) via in-line UV-Vis characterisation led to the discovery of five classes of nanoparticles by only needing to perform ca. one thousand from a total of ca. >10²³ possible experiments. The platform optimises the nanostructures with desired optical properties by combing experiments and scattering simulations to achieve a yield of up to 95% and the synthesis code is outputted in a universal format using Chemical Description Language (χDL) together with the analytical data to produce a unique digital signature to aid and confirm that the synthesis is universally reproducible.

The present inventor has previously described in WO 2013/175240 the use of an automated flow system for exploration of a chemical space, and for identifying a product having meeting or exceeding a user specification, as judged against a fitness function. The flow system runs continuously to prepare reaction products in series, with each reaction mixture analysed by an inline analytical system. Based on the analytical results, a suitably programmed control system is guided by a genetic algorithm to select future chemical inputs, optionally together with physical inputs, into the flow reactor. In this way, chemical and physical inputs that are associated with products having a higher fitness rating may be amplified for the preparation of further products, with the expectation that this amplification will lead to products having enhanced fitness functions.

The genetic algorithm is also capable of making random changes—or mutations—within the inputs to allow the system to explore other regions of the available chemical space, and to avoid the system becoming trapped in local areas of maxima.

This system does not allow for the products of any reaction to be used as an input for any subsequent reaction. Rather, the system is limited by the chemical inputs that are initially proved to it, and it cannot make use of any of the products that it has made itself. The available product space is therefore limited. In contrast, the methods of the present case allow a selected product from one series of reactions to serve as a chemical input into a subsequent series of reactions. It follows that the use of such a selected product allows for an expansion of the available chemical space into which the systems of the invention can explore.

In the preferred embodiments of the invention, a further expansion of the available chemical space is achieved through the selection of a plurality of different products from a series of reactions, and each of the selected products may be used as chemical inputs into a plurality of subsequent series of reactions. In this way, the chemical space is vastly expanded.

Furthermore, the system in WO 2013/175240 does not allow for any controlled expansion and optimisation of the chemical space. In the preferred embodiments, the present case provides a series of reactions that are intended to expand the available reaction space, and once a plurality of products having descried fitness values are identified, the system may then use those products to prepare optimised products.

### Autonomous Exploration Apparatus

The present invention provides an autonomous exploration apparatus for use in the methods of exploration described herein.

The autonomous exploration apparatus includes a chemical synthesiser controllable by a suitably programmed controller. The controller selects the chemical inputs optionally together with physical inputs for use in a series of reactions undertaken by the chemical synthesiser. The controller may also select methods for the work-up and purification of reaction product mixtures, for example to prepare a product for analysis, and for subsequent use in a later series of reactions.

The autonomous exploration apparatus also includes an analytical unit for measuring the characteristics of products produced by the chemical synthesiser. The analytical results are provided to and analysed by the controller, and the controller reacts to the analytical results by selecting an appropriate reaction product to act as a seed for subsequent series of reactions, and additionally selecting chemical and physical inputs for the subsequent series of reactions within the autonomous exploration process. The selection of products for use in a series of reactions, and the selection of chemical inputs optionally together with physical inputs for use with those products, is instructed by the controller.

The controller itself may be a suitably programmed computer, which is in communication with the chemical synthesiser and the analytical unit.

The automated chemical synthesiser comprises a series of reaction spaces, with each space for independently performing a chemical reaction. Each reaction space may be independently supplied by suitable chemical inputs, optionally together with physical inputs. The automated chemical synthesiser may have at least 10 separate reaction spaces, such as at least 20 separate reaction spaces, each of which may be independently serviced with chemical inputs, optionally together with physical inputs.

The automated chemical synthesiser is provided with apparatus for conducting a chemical reaction. This may include, amongst others, heating elements, stirrers, shakers, and light sources.

Chemical inputs, such as reagents, solvents and catalysts are deliverable to each reaction space. The chemical synthesiser may be provided with an array of reservoirs for supply of chemical inputs to each reaction vessel. The timing of addition of chemical inputs, and the absolute and relative amounts of each chemical input is determined by the controller which instructs the synthesiser as appropriate. Similarly, the application of physical inputs, to the chemical inputs or to the reaction vessel, and the timing and duration of such, where needed, is also determined by the controller which instructs the synthesiser as appropriate.

The chemical synthesiser typically has a modular construction, and additional modules may be included within the synthesiser as needed. For example, additional chemical and physical input sources may be added to the synthesiser as needed.

The chemical synthesiser cooperates with the analytical unit, and the analytical unit may be provided as a module integrated with the chemical synthesiser. Here, the analytical unit has ready access to the reaction products, which allows for rapid analysis of those products.

Alternatively, the analytical unit can be separate from the chemical synthesiser, and the chemical synthesiser may make a reaction product available to the analytical unit for analysis. The product separator of the chemical synthesiser may be used for this purpose.

Each reaction space may be a reaction vessel, such as a reaction flask.

A reaction space is serviced by a product separator to separate a reaction product from a reaction mixture within a reaction space.

A reaction space may also be serviced by a waste unit, which unit removes any reaction mixture from a reaction space after reaction and after product separation, and optionally cleans the reaction space to ready if for use in a subsequent reaction, for example in a subsequent series. The recycling of reaction spaces in this way allows the chemical synthesiser to remain relatively compact, as a discrete reaction space is not required for each and every reaction that is to be performed.

Thus, the apparatus may include a waste unit for removal of material from a reaction space. A reaction vessel may be adapted to allow for removal of material with, for example to include drainage lines for the waste unit. The waste unity may be provided with physical and chemical materials to allow removal of reaction vessel contents, and this may include a vacuum supply, and a fluid supply such as gas or solvent supply to move the reaction space contents.

The product separator may cooperate with the waste unit to allow reaction products to remain within a reaction vessel for use in a later reaction within a subsequent series of reactions. However, it is preferred that the product separator removes a selected product from a reaction space, and them makes the product available to a reaction space as needed for reaction in a subsequent series of reactions.

In a preferred embodiment of the invention, the automated chemical synthesiser comprises a Geneva wheel. The chemical and physical inputs, the product separator, the analytical unit and optionally the waste unit may be arranged around the Geneva wheel to allow for the reaction spaces to be presented in sequence between various units.

The Geneva wheel is a series of reactions spaces, typically arranged in sequence in a circular arrangement, where each reaction space is moveable in turn between a plurality of stations. The stations may include one or more of (i) one or more reagent supply stations; (ii) a purification station; (iii) an analysis station; (iv) a sample removal station; and (v) a reaction vessel cleaning station.

A Geneva wheel allows multiple reaction vessels to be serviced at any one time, and without the need for the system to wait for a batch of reaction products to be prepared.

The product separator is provided for the collection of products from the reaction space. A product may be removed from a reaction space for analysis. Additionally or alternatively, a product may be removed from a reaction space for later supply to a reaction space for reaction in a subsequent series of reactions.

The collection of a product from a reaction space may include the at least partial purification of the product from other components of the reaction product mixture, such as one or more of a solvent, unreacted reagents, catalysts, and by-products. The product may be collected following a work-up of the reaction mixture. The work-up may include filtration phase separation, concentration and drying amongst others.

The product separator may make available a product for the analytical unit to analyse.

The product separator is under instruction from the controller. Under such instruction, the product separator may provide a product, which may be a selected product, to a reaction space for use as a reagent in a series of reactions. Thus, the product separator delivers the seed for a subsequent generation of reactions.

In one embodiment the product separator is provided with a storage unit for storing a reaction product. A reaction product is deliverable to the storage unit, for example after at least partial purification from a reaction product mixture, and is then made available from the storage unit to a reaction space as needed.

The product separator is capable of supplying a selected product to multiple reaction spaces, such as multiple reaction spaces in a series. The product separator is able to divide the selected product into a required number of portions for delivery to the required number of reaction spaces. Typically, a selected product may be taken up into a liquid, giving a suspension or solution as appropriate, and this liquid mixture may then be distributed across the requisite reaction spaces using standard fluidic techniques. The concentration and the amount of fluid may be controlled under instructions from the controller.

The product separator may cooperate with the input array to formulate a selected product suitable for use in a subsequent series of reactions. Thus, the product separator may be suppliable with chemical inputs to generate an appropriate formulation for the selected product.

Alternatively, the product separator may be provided with separate chemical inputs to formulate a selected product for delivery to a reaction space.

The controller is a suitably programmed computer that is capable of controlling an automated chemical synthesiser. The use of such controllers within chemical robotics is well known.

The controller also receives analytical information from the analytical unit, and the controller is capable of analysing the analytical information, and using that analytical information to make decisions regarding the selection of a product, and the use of that product in a subsequent series of reactions.

### Staged Exploration and Optimisation

The present invention provides a method for the exploration of a chemical space. The exploration is a series of chemical preparations, where structural and synthesis information from an earlier stage in the exploration may be used to inform and guide the development of the products in subsequent exploration stages. Thus, the exploration may be regarded as a staged exploration in that the later steps in the exploration are dependent upon the performance of earlier steps.

In the present case, a product is prepared in one stage of the exploration, and that product may be used as a starting material in a later stage of the exploration. In this way, the product is a seed for the generation of later products in the exploration. The product may therefore carry structural information, which may be associated with desirable physical and chemical characteristics, into later generation syntheses, and therefore later generation products.

A new stage in the exploration method, which is referred to as a generation, is characterised in that it uses a product prepared earlier in the exploration.

Thus, the exploration method comprises:
(i) a first stage, where a first series of reactions is performed, and an analysis of the reaction product for each reaction in the first series is performed, and a first product from the first series of reactions is selected; and
(ii) a second stage, where a second series of reactions is performed, where the selected product from the first stage is provided as a chemical input to each of the reactions in the second series, and an analysis of the reaction product for each reaction in the second series is performed, and a product from the second series of reactions is optionally selected,
wherein the first and second stages are performed autonomously, and the selection of a product in the first stage comprises the comparison of the products from the first series of reactions against a fitness function, where the selected product has a superior fitness compared with one or more other products in the first series, and
each reaction in the first series differs in one or more chemical and/or physical inputs, and each reaction in the second series differs in one or more chemical and/or physical inputs.

The second stage may be referred to as a generation coming after the first stage generation.

In the first stage, a common chemical input, such as a reagent, may be provided. This common chemical input may itself be a seed. Thus, an original seed may be externally supplied to the system to initiate or guide the exploratory synthesis undertaken by the autonomous exploration apparatus.

Where the first stage uses a seed, this may also be prepared by the system itself in a preliminary synthesis under the control of the controller. The controller may be suitably programmed to prepare a range of seeds, which may be known compounds. Thus, the controller may store synthesis instructions for the preparation of a seed, and the synthesis of the seed may be executed for the purpose of supplying a first stage with a common chemical input.

In one embodiment of the invention, the first stage comprises a series of reactions where a chemical input is common between each reaction in the series. The chemical input may be a seed prepared prior to the performance of the first stage. Thus, the first stage of the method uses a starter reagent as a chemical input to each of the reactions in the first series. Here, the starter reagent refers to the common chemical input in the first stage.

A selected product may be referred to as a seed for use in a next generation series of reactions. The selected product may be a template for the subsequent synthesis steps.

The methods of the invention are particularly suited to the preparation of nanomaterial products, such as nanoparticles. The methods of the invention may also be used more generally to prepare other materials, and may have be used in the preparation of new compounds.

The method of the invention may comprise repeating first and second stages multiple times, where a selected product from each repetition of a second stage is used as a common chemical input in a subsequent performance of a first stage.

In the first stage more than one product may be selected, and each of the selected products has a superior fitness compared with one or more other products in the first series. Here, one of the selected product is used in a subsequent series of reactions, and each of the other selected products is used in a subsequent separate series.

Thus, in one embodiment of the method:
(i) in the first stage, a first series of reactions is performed, and an analysis of the reaction product for each reaction in the first series is performed, and a first and second product from the first series of reactions is selected; and
(ii) in the second stage, a second series of reactions is performed, where the first selected product from the first stage is provided as a chemical input to each of the reactions in the second series, and an analysis of the reaction product for each reaction in the second series is performed, and a product from the second series of reactions is optionally selected, and
the method further comprising:
(iii) a third stage, where a third series of reactions is performed, where the second selected product from the first stage is provided as a chemical input to each of the reactions in the third series, and an analysis of the reaction product for each reaction in the third series is performed, and a product from the third series of reactions is optionally selected,
wherein the first, second and third stages are performed autonomously, and the selection of the first and second products in the first stage comprises the comparison of the products from the first series of reactions against a fitness function, where the selected products have a superior fitness compared with one or more other products in the first series, and
each reaction in the first series differs in one or more chemical and/or physical inputs, each reaction in the second series differs in one or more chemical and/or physical inputs, and each reaction in the third series differs in one or more chemical and/or physical inputs.

In each of the second and third stages a product is optionally selected. The selection of a product in the second stage comprises the comparison of the products from the second series of reactions against a fitness function, where the selected product has a superior fitness compared with one or more other products in the second series. The selection of a product in the third stage comprises the comparison of the products from the third series of reactions against a fitness function, where the selected product has a superior fitness compared with one or more other products in the third series.

Where more than one product is selected, the selections are a subset of all the available products in a series. Typically, the selected products make up a minority of the all the available products in a series. With practical considerations in mind, and with respect to the selection of the most promising products having the best fitness functions, a selection may be made of one, two or three products only.

A selection of a plurality of products in a stage may select those products having superior fitness functions. Where a selection is made, the system may also make a selection of plural products that are seen to have the greatest diversity, such that the subsequent series of reactions may expand upon that diversity to fully explore the available chemical space.

Here, it is recognised that the system may be blind to structure, as it looks to identify products that have the best fitness values. However, a consideration of structure may also be helpful and important, as it can provide an understanding for the relationship of the product to its fitness value. Accordingly, structural information may also assist in developing a model for the products that in turn allows for the selection of certain chemical and physical inputs for the stages in the method. Analytical information on structure may be used advantageously to select products having significant differences, and these different products may be used to expand the available chemical exploration space, with the desire to identify novel optimised products.

In the present method, the purpose of the first, second and third steps together is to provide for an extensive exploration of the chemical space. Subsequent steps, where present, may also be intended to provide for an extensive exploration of the chemical space. The selection of a plurality of products for use in a subsequent series of reactions may allow for the most rapid expansion and exploration of the available chemical space.

Following an initial expansion and exploration of an available chemical space it is expected that various lead products will be identified. These lead products are products having the most promising characteristic, scoring highly against the fitness function. These leads are then selected for optimisation. Here, there is no need for the selection of multiple products to expand into the chemical space. Rather a product may be selected for exploration of the space immediately around it.

Thus, whilst the initial exploration stage provides for expansion, the optimisation stages provide for finer control.

The method of the first aspect of the invention may comprise one or more additional optimisation stages, performed after the second stage, and optionally the third stage. The method further comprises:
(iv) an additional optimisation stage, where a series of reactions is performed, where the selected product from an earlier stage, such a selected product from the second stage, or a selected product from the third stage, is provided as a chemical input to each of the reactions in the additional series, and an analysis of the reaction product for each reaction in the additional series is performed, and a product from the additional series of reactions is optionally selected,
wherein the additional stage is performed autonomously, and the selection of a product in the additional stage comprises the comparison of the products from the additional series of reactions against a fitness function, where a selected product has a superior fitness compared with one or more other products in the first series, and
each reaction in the additional series differs in one or more chemical and/or physical inputs.

An additional stage may follow from one or both of the second and third stages. This in turn follow from the first stage. An additional stage may be performed after an earlier preceding stage, and the number of additional stages that may be undertaken, with each additional stage making use of a selected product from an earlier stage, is not limited. Thus, a series of staged reactions may be performed until such time as an optimised product is obtained having the meeting the desired fitness function is identified, or until such time as the product having the maximum fitness value is found.

The methods of the invention may use both exploration steps and optimisation steps in any order and sequence, and may use mixtures of both within any generational line of reactions.

Typically, the methods of the invention will initially use exploration steps, where more than one product is selected from a series of reactions for subsequent use in a plurality of stages, and those more than one products are each used in subsequent later stages. From those later stages, each being a series of reactions, one product may be selected and subsequently used in an additional stage.

Thus, the methods of the invention may include a repeat of the first, second and third stages, where each reaction in the first stage series of reactions uses a selected product from an earlier stage in the method.

A stage of the method includes the step of performing a series of reactions, each of which differs in at least one chemical input or optionally one physical input. A series of reactions may contain at least four different reactions, such as at least 10, such as at least 20 reactions.

Each reaction in the series a reaction product mixture, which is analysed. The products in the reaction mixtures may differ. However, some reaction mixtures may have products in common. Where all the products are the same, any one of the products may be used in a subsequent step, where a product is selected.

The number of reactions performed in a series is not necessarily limited by the number of available reaction spaces. Where the number of reactions is greater than the number of reaction spaces, an initial fraction of the reactions in the series may be performed in the available spaces, and the remaining fraction of reactions may be performed as and when reactions spaces become available.

The nature of the reactions performed in any series is not particularly limited and may comprise reactions forming covalent or non-covalent bonds, or a mixture of both. The reactions may include self-assembly.

The selection of a product having a superior property, shown by the fitness function, may allow the perpetuation of a desirable characteristic of early generation products into higher generation products.

In an embodiment of the invention, a single product is selected from the series of products, and this product may be an elite product. An elite product may be a product having the highest fitness function amongst the series of products.

In a further embodiment, two or more, such as two, products are selected from the series of products. One of those products may be an elite product. Any other products selected may be those products having the highest fitness functions after the elite product.

An additional series may make use of a selected product from a stage that immediately precedes it (a parent stage). In an alternative embodiment, an additional series may make use of a selected product from a preceding stage that does not immediately precede it (such as a grandparent stage).

The methods of the invention may include the step of isolating a plurality of products that are produced in a series of reactions. Typically, one of those products is used in a subsequent series of reactions. Later, another of the products may be then used in a subsequent series of reactions. It may be the case that an initially promising product-having a high fitness value, such as being an elite product-is taken forward into a later generation, but further exploration may lead to products that have reduced fitness values or products that cannot be satisfactorily optimised. In this situation, the methods of the invention may then revert to an alternative product from an earlier generation, and that product may then be used in a subsequent series of reactions. Thus, where one product is selected from a series od reactions, one or more other products that are not selected may anyway be saved for possible later us in the exploration and optimisation steps described herein.

The methods of the invention include those where the method proceeds continuously within each stage. Thus, the method is performed without halt until at least the final product in the series is prepared. This has the benefit of reducing downtime in the methods of the invention.

The methods of the invention include those where the method proceeds continuously from one stage to a following stage. Thus, the method is performed without a halt between the stages. This has the benefit of reducing downtime in the methods of the invention.

The methods of the invention include those, where a part of a later series of reactions may be performed coincident with the performance of a part of an earlier series of reactions. Thus, a subsequent series of reactions may be initiated for a later stage whilst the method is still working to finish an earlier series of reactions within an earlier stage. In this situation a first product may be selected from the earlier series of reactions before that series of reactions is complete, and that selected first product may then be used as a chemical input in a subsequent series of reactions. Here, during the performance of a first series of reactions, a first product having an excellent fitness may be identified early in the series of reactions, and that first product may then be selected for use in subsequent stages. The earlier series of reaction is nevertheless completed, as further products having desirable fitness functions may still be produced, and they may be selected for use in alternative subsequent stages.

In each stage of the method, there is a variety in the chemical inputs, optionally together with the physical inputs, across the series. Thus, each reaction in a series differs in one or more chemical and/or physical inputs. With a variation in the chemical or physical condition of each reaction there is the possibility of accessing a variety of different reaction products.

The controller may select chemical and physical inputs in expectation of achieving a variety in the reaction product mixtures. In some embodiments, a difference in the reaction product will be inevitable following differences in the choice of chemical inputs, such as the choice and range of reagents for use in the reaction.

Of course, in any reaction series multiple identical products or product mixtures may result, and this may be apparent to the controller from the analytical results. The controller may learn from this, and future syntheses may be adapted to prevent the likely replication of reaction products across a series, or indeed across separate series.

Where a reaction series produces multiple products having similar properties, and having similar fitness functions, such that the product is suitable for use in a subsequent series, the controller may choose amongst these for a selected product for use in the subsequent series.

In one embodiment, the exploration may be performed as a sequence of synthesis stages. In each stage, a sequence of reactions is performed to give a product library, with the analysis of each product in that library. The next stage in the exploration is undertaken only once the previous stage is completed, and the products analysed. In this way, the exploration may be regarded as a sequence of batch syntheses.

However, in the preferred embodiments of the present case, the exploration may be run continuously or semi-continuously. Thus, there is no significant down-time in the exploration, leading to a more rapid expansion of the chemical space.

The preferred methods of the invention make use of spectroscopic analysis, and particularly those analysis that can be performed and analysed quickly. Favoured example here are IR and UV-vis spectroscopies.

### Selection and Fitness Function

The methods of the invention include a step of selecting a product from a series of products produced in a reaction series. That product is then used as a chemical input in a subsequent series of reactions.

The selection of a product from a series of products is controlled by the controller, which responds to the analytical information recorded for each product, and uses that analytical information to compare each product against a fitness function. A selected product has a superior fitness compared with one or more other products in the series. In one embodiment, the selected product has the highest-ranking fitness compared against the other products in the series.

Products having a poor characteristic, as judged by having a low fitness function, may simply be discarded, for example using the waste unit. Products having a high fitness, whether measured absolutely or relatively, may be selected, and may be stored for later use in the exploration method, or may be used in the next series of reactions.

The fitness function is developed from the requirements of a user for a product having a particular physical or chemical characteristic. The specification set by the user is ultimately translated into a physical product that has a fitness function that meets or exceeds the characteristics that are deemed desirable by the user.

The use of fitness functions is known from the inventor's earlier work in WO 2013/175240, the contents of which are hereby incorporated by reference in their entirety.

The analysis methods in the methods of the invention are selected for the fitness function.

The methods of the develop a series of products from a reaction series. Each of these products is analysed and its fitness value is determined. A product may be selected from amongst all the products produced in a series for use in a subsequent stage of the method. A selected product is one having a superior fitness compared with one or more other products in the same series. The selected product may be product having the highest fitness value. This product is the elite. Alternatively, the selected product is simply a product not having the worst fitness value.

Where multiple products are selected, it is preferred that this is a subset of all available products in a series. Where a plurality of products is selected, one of those may be an elite, and the other products may be those products ranked in order below the elite.

Where multiple products are selected, they may be those products having the highest fitness values.

In some embodiments, multiple products maybe selected each having a superior fitness compared with another comment product in the series. Optionally where multiple products are selected these products may be selected on the basis that the selected products having structural or chemical dissimilarity. Here, products may be considered to be dissimilar to one another if they have more structural or chemical similarity to another product that is not selected. Suitable analytical units may be provided to analyse chemical and/or structural features, and to allow a comparison of structural and chemical similarity.

In some circumstances, an analytical method may directly measure the physical or chemical characteristic that is required by the user. In other embodiments, an analytical method may measure a physical or chemical characteristic of a product that is connected, directly or indirectly, with the physical or chemical characteristic that is required by the user.

A physical property of the product may be a characteristic selected from the group consisting of:
optical property,
mass property,
electrochemical property, and
rheological property.

A physical property of the product may be a characteristic selected from the group consisting of:
Colour
Refractive index
Optical rotation
Absorbance maximum; Wavelength of absorbance maximum
Emission wavelength
Absorbance value at set wavelength
Molar extinction coefficient
Fluorescence, presence, yield and/or wavelength of
Photoluminescence, presence, yield and/or wavelength of
Non-linear optical properties, presence of
Retention time (for example, in a HPLC)
Mass
Polydispersity
Isotopic distribution
Elemental composition, for one or more than one element
Reduction/oxidation potential
pH, for example of an aqueous product mixture
Elongation
Flow
Storage modulus
Loss modulus
Viscosity
Morphology, including crystallinity
Stoichiometry
Size, for example diameter of particles, or pore or cavity size
Shape
   and optionally together with the above, surface texture, harness and surface tension,
Conductance
Magnetic properties
Melting point, including Tg
Boiling point
Solubility, for example in a set solvent or series of solvents

The chemical property of the product may be a characteristic selected from the group consisting of:
pKa
KD
IC₅₀
MIC (minimum inhibitory concentration)
catalytic ability

### Target Products

The present invention provides methods for exploring chemical space, using a seeding approach to develop higher order products.

In preferred embodiment, the invention provides methods for the exploration of a nanoproduct chemical space, such as the preparation of nanoparticles, nanofibers and nanorods. A nanoproduct is a structure may be one having a largest dimension of no more than 500 nm, such as no more than 100 nm. A nanoproduct is a structure may be one having a smallest dimension of 0.05 nm or more, such as 0.1 nm or more.

The dimensions of a product may be determined, for example, by microscopy, such as TEM, as described herein.

However, other product materials may be prepared by the methods described herein, as will be apparent to those of skill in the art.

### Seed

In the methods of the present invention, a seed is used for the preparation of products within a generation of the staged exploration.

A seed is a product from an earlier generation. Typically, a seed is taken from the immediate precursor generation (parental generation). In the methods of the invention the seed is a selected product from a series of reaction product mixtures produced in a series of reactions, such as the first series, the second series or subsequent series of reactions.

Thus, a seed for one generation that is a series of reactions may be a selected product from an earlier generation that is an earlier series of reactions.

However, in some embodiments, a seed may also be a product from a generation that is two or more stages earlier in the exploration.

In the first stage of the exploration, an initial seed may be provided as the basis for the synthesis. Thus, a suitable seed is prepared independently from the methods described herein, and the seed is made available for use.

Alternatively, the first stage of the method may include the step of preparing a seed that is intended for use in subsequent generations.

In some embodiments, a plurality of seeds may be used in a single reaction, although this is less preferred. Such seeds may be different products from within the same series of reactions (the same generation), or alternatively seeds may be different products selected from across two or more series (two or more generations).

Multiple seeds may be used to explore the possibilities for more complex assemblies of products, thereby providing the potential to access higher order product spaces.

In any one generation, a single, common seed may be used for all syntheses. However, this is not essential. In alternative methods, two or more products from an earlier generation may be used as seeds in an exploration stage.

In this way, a particular generation may have a common immediate ancestor.

The use of multiple seeds across an exploration step can be advantageous, as it permits a rapid expansion of the chemical space, and it may also maximises the opportunities for identifying novel products, or for identifying new processes for the preparation of known products.

The selection of multiple seeds from a single generation may also account for situations where multiple products in that generation have desirable characteristics linked to the fitness function. Rather than selecting a single seed for exploration, the system may allow two or more seeds to be propagated into later generations.

In this way, a particular generation may not have a common immediate ancestor.

A seed for use in later generations may be selected for use as such on the basis of its characteristics as measured against the fitness function.

In the preferred embodiments, of the present case, a spectroscopic characteristic of a product is used as the basis for establishing the characteristics of a product for measure against a fitness function. Thus, a seed may be chosen on the basis of its spectral profile, which may match closest to the desired profiles when compared against other products prepared within a generation.

### Reaction Conditions

The methods of the invention provide multiple series of reactions, where each series of reaction represents a generation of syntheses within the exploration method.

Typically, each reaction within a series is unique, in that it does not replicate a reaction previously performed across the exploration space, across the series and across any earlier generation. A specific reaction may be distinguished from other reactions by any a chemical or a physical reaction input.

Thus, where a reaction differs in a chemical reaction input, one or more of a reagent, catalyst or solvent may differ from that used in an earlier reaction.

Where a reaction differs in physical reaction input, one or more of a temperature, pressure, incident light intensity or wavelength, amongst others, may differ from the physical conditions used in an earlier reaction.

In one embodiment, a reaction condition may differ from others in the identity or amount of the seed. This may be the only difference, or this difference in seed may be combined with one or more differences in the chemical or physical inputs.

The system of the invention determines the reaction conditions for use in each reaction. The choice of reaction conditions for a specific reaction may be determined

### Physical Inputs

The method also includes the provision of one or more physical inputs which are made available to the reaction space, or to a chemical input prior to that chemical input entering the reaction space.

A physical input is intended to refer to an input that is not a material such as a reagent, catalyst, solvent, or a component. A physical input may refer to, for example, an input that modulates temperature, such as the temperature of a particular chemical input, or the temperature of the fluids in the reaction space. A modulation in temperature may refer to a physical input than can raise and/or lower temperature. A series of temperature inputs may be provided that is a gradient of temperature increase and/or decreases. The range of temperature inputs may be limited by the boiling and freezing points of the fluid chemical inputs supplied to the reaction space, and the fluid product output. It is noted, however, that the reaction space may be suitably pressurised thereby to effectively alter the boiling and freezing points of the fluid chemical inputs. In this way a greater range of temperature inputs may be supplied to the system.

Temperature inputs may be used to initiate reagents or favour certain reaction pathways. Temperature inputs may also be used to investigate the stabilities of the chemical input and product output.

The physical input may be light. A series of light inputs may be provided that differ in one or more of intensity, wavelength, exposure time and spectrum. Light inputs may be used to initiate reagents or may be used to favour or alter certain reaction pathways. Light inputs may include UV-vis inputs. The physical input may be microwave radiation.

The physical input may be ultrasound. Such may be useful for the generation of reagents or products. Ultrasound may also aid the dissolution of material.

The physical input may be pressure. Pressure changes may be used to alter, for example, solvent boiling points.

The physical input to the system may be a process related input for the reaction mixture. Thus, the input may be a time limited feature for reaction or admixture. After a set time, the reaction mixture may be analysed and the product quantified. Thus, reaction time may be an input. Similarly, other process features such as concentration and ratio of chemical inputs, such as the reagent and catalyst chemical inputs, may be a physical input.

### Chemical Inputs

The reference to a chemical input is a broad reference to any material, which may be a reagent, catalyst, solvent, or a component, that may allow the preparation of a product.

Each chemical input may be deliverable independently from other chemical inputs to a reaction space. Thus, there is freedom on the system to deliver a specific reagent, solvent or catalyst independently of other components.

The chemical input is provided as a solid, or in a fluid for transfer to the reaction space.

Where the material is a fluid, it may be supplied in this form to the reaction space. Alternatively, the material may be diluted, dissolved or suspended in a fluid for delivery to the reaction space. Thus the material may be in solution or suspension. The fluid that dissolves or suspends the material is not particularly limited, and may be water or an organic solvent, for example. The fluid may be independently deliverable to the reaction space. The fluid is also used to provide separation between individual combinations of chemical inputs that are supplied to the reaction space thereby preventing contamination between different combinations.

The identity of the chemical inputs will be dependent upon the reaction and formulation steps that are to be employed, and may be limited-though not necessarily-on an intended exploration space. Whilst the present invention allows a autonomous system to explore a product space, a user may anyway provide boundaries to that space by way of choosing a set of reagents, catalysts, solvents, and components, which may thereby limit possible reaction and formulation pathways.

Within those confines, the present invention nevertheless allows the autonomous system the possibility of exploring a broad range of product space. The examples in the present case demonstrate the breadth of structural complexity that is available in a nanomaterial synthesis employing a small range of chemical inputs.

In some embodiments, one or more, such as two or three, chemical inputs may be regarded as essential. Thus, these inputs are always provided into the reaction space. The alteration of other chemical and/or physical inputs provides the variety in the combination that permits an exploration of the product space. The number of essential inputs is less than the total number of available inputs, and is preferably considerably 5 less than the total number of available inputs.

An input may be essential if it is necessary for providing a necessary component of the product, such as a structural component, including a particularly type of bond, or a necessary activity of the product. Other inputs are available and are variable in order to investigate other conditions for preparing the particular product.

A chemical input may be a reagent. A range of reagents may be provided that differ in their structure and functionality.

A chemical input may be a catalyst. A range of catalysts may be provided that differ in their activity, selectivity, or morphology.

A chemical input may be an acid or a base. A range of different acids and bases may be provided, where the acidity differs. Organic and inorganic acids and bases may be selected.

Weak and strong acids and bases may be provided.

A chemical input may be a solvent. Organic solvents and water may be used. A range of non-polar, protic and aprotic solvents may be provided. In one embodiment, water is provided as a chemical input.

A chemical input may be a salt. A range of different salt forms of a particular component may be used. A range of organic and inorganic salts may be provided.

A chemical input may also be a gas. In some embodiments, a chemical input may be an inert gas, such as nitrogen or argon, to supply to the reaction space. In other embodiments, the chemical input is a reaction gas, such as hydrogen, oxygen or carbon dioxide.

A chemical input may be an input that is for useful in the work up of a reaction product, or is useful for quenching a reaction. Such inputs may be provided to the reaction space at some time period after the other inputs have been combined, thereby to quench a reaction or to permit the work up and possible isolation of product material.

In an additional embodiment, one chemical input is a reagent, and that reagent may be in common for each of the chemical reactions within a series. Inf further embodiments, there be two or three chemical inputs in common for each of the chemical reactions within a series. However, is still the case that each reaction in a series differs in one or more chemical and/or physical inputs from each of the other reactions in the series.

Where, a second series of reactions is performed after a first series of reactions, the second series makes use of a common reagent, which is a selected first product from the first series. Where, a third series of reactions is performed after a first series of reactions, the third series makes use of a common reagent, which is a selected second product from the first series. In addition to the first or the second product each of these series may have a further common reagent.

The concentration of a material within a solution or in a suspension will be selected appropriately by the system. The effective concentration of the material in the reaction space will depend on the concentration of that material within its individual chemical flow and the volume of other chemical inputs with which it is combined in the reaction space. These volumes are dictated by the flow rates of each of the inputs, which may be varied as appropriate, to alter the effective concentration of a material in the reaction space. Such techniques will be familiar to those with an understanding of flow chemistry techniques.

Preferably, the material that is present as a chemical input is stable. The autonomous system may require that a chemical input is stored for a time before it is used. Therefore, it is preferred that the chemical input does not decompose in this time. Where appropriate, a chemical output may be stored under an inert atmosphere, may be stored under anhydrous conditions or may be stored at reduced temperature, as required.

In one embodiment, there is provided 5 or more, 8 or more, 10 or more, or 15 or more chemical inputs. For example, the flow chemistry system may comprise a number of controllable syringes equal to the number of specified chemical inputs.

From time to time it may be necessary to replenish a chemical input. The process of the invention need not be halted to allow such replenishment, and the chemical input may be replenished at such a time as it is not required as an input into the reaction space. The control system may be suitably programmed to predict the time at which a chemical input will become depleted. An operator may be warned accordingly. The control system may also be suitably programmed to factor in to the decision making and control process the unavailability of an input owing to replenishment. The control system can continue to produce products using inputs other than the input that is being replenished.

The number of chemical inputs may be one, though in this embodiment the number of physical inputs, which may bring about a change in the chemical input, will be large.

In one embodiment, there is provided two or more, three or more, four or more, five or more, six or more, ten or more, twenty or more chemical inputs.

### Analysis and Analytical Unit

Preferably, the methods of the invention include the step of analysing the products in the series of reactions. The apparatus of the invention is provided with an analytical unit for analysing the products.

The analytical results are used as the basis for selecting a product from the series of reactions, which product is then used as a chemical input into a subsequent series of reactions. The analytical results obtained by the analytical unit may be provided to the controller. The controller receives analytical information from the analytical unit, and to compare the analytical information for reaction products against a fitness function.

The method of the invention includes the performance of a series of reactions, and these may be performed across the series of reactions spaces of the chemical synthesiser. The product reaction mixtures of each reaction in the series may be analysed.

In one embodiment, the product reaction mixture may be analysed. Here, it is not necessary to perform any substantial work-up and purification of the product mixture.

Alternatively, a product of a reaction may be analysed after the reaction product is purified from the reaction mixture, however this is not essential, as characterising analytical information may be obtained from a crude reaction mixture.

The analysis undertaken and the manner in which it is undertaken will depend upon the nature of the chemistries employed in the reaction series, and the type of analytical methods for use.

The analytical unit is adapted for interaction with the chemical synthesiser. The analytical unit is provided for the purpose of analysing a product produced in a reaction space, or obtained from a reaction space. The analytical system is in communication with the controller.

Thus, the analytical data is provided to the controller for comparison against the fitness function. The analytical unit is automated.

Thus, the system is adapted such that it is capable of receiving a product mixture, which is optionally at least partially purified mixture, analysing the product mixture or any product extracted from it, and supplying the analytical data to the controller.

Additionally, the analytical unit may also be used to monitor the chemical inputs into the reaction space, and the progress of the product formation within the reaction space.

In this way, the analytical system may also be useful to monitor reaction progression, and the system may be adapted to make an intervention to a reaction where this is deemed appropriate. For example, the reactions for performance may require, or desirable products may be formed under, certain reactions conditions and the maintenance of those conditions throughout the synthesis. For example, temperature and pH values may be important. The analytical system may monitor the system and the controller may instruct the application of chemical and/or physical inputs as needed.

The analytical unit may be integrated with the chemical synthesiser.

The analyses for use in the present case include those based on IR, UV-vis, Raman and NMR spectroscopies, and the like. The fitness function for the target product may be developed to make of standard and easily accessible analytical devices.

In order to develop high throughput systems, where the methods of the invention may develop in relatively short time, it is beneficial to use efficient in-situ and on-line analytical techniques, such as pH, UV-Vis, Raman and IR.

Preferably, the analytical technique is passive or non-destructive. Thus, the sample may be tested without requiring any physical or chemical degradation of a product. In this way a sample may be tested by many different methods, if needed.

Where a product having a high fitness function is identified, such as an elite product, it may be useful to further test that product in order to confirm the initial positive identification. Additionally, it may be beneficial to conduct further analytical tests on a selected product to confirm its usefulness. These further analyses may require larger quantities of material or may be of a time-consuming nature that is not suitable for use together with the relatively speedy exploration steps described herein. Such techniques may therefore be conducted on compounds that are identified as desirable by way of other, more immediate analytical techniques.

A selected product may be provided to analytical devices such as mass spectrometers, NMR spectrometers and microscopes. The product may be a selected product obtained by the product collector.

The analytical system may test a particular reaction mixture in a reaction space. The results from the analytical analysis may be supplied to the controller which will respond to the output by altering the inputs into the reaction space. Where the analysis is rapid, the control system will be able to respond rapidly and will be capable of formulating the next series of inputs in direct response to the output.

In one embodiment, the analytical system has a UV-vis and/or an IR detector. In one embodiment, the analytical system has a pH detector.

### Control and Exploration

The present invention provides an intelligent exploration of chemical space. Thus, the reaction conditions chosen for every reaction—including the choice of a seed—are not made completely randomly.

The controller is suitable programmed with an AI algorithm to analyse analytical data and to select products for use in a series of reactions, and to select future chemical inputs, optionally together with physical inputs, for use with the selected product a series of reactions.

The system uses its understanding of reaction products and their fitness to plan future series of reactions.

The system may also operate to expand the available chemical space by deliberately making choices, that is selecting products and/or selecting chemical inputs optionally together with chemical inputs, that are not associated with high fitness values. The controller may make such choices in order to provide a mutation into the methods, thereby allowing the exploration of alternative chemical space. These selections may be random, or they may be selected by the controller.

### Library

The present invention also provides a library of products obtained and obtainable from the methods of the invention.

A library is a collection of products, such as nanomaterial products, produced by the methods described herein. A library may be provided with an electronic instruction set for one or more, such as each, product, which instruction set is an experimental description of a method for obtaining the nanomaterial product using an automated chemical synthesiser.

A library which is a collection of a plurality of of products obtained or obtainable from the methods of the invention. The library may contain all of the products produced in a method. However, in other embodiments the library may contain only selected products from each of the reaction stages.

### Other Embodiments

Each and every compatible combination of the embodiments described above is explicitly disclosed herein, as if each and every combination was individually and explicitly recited.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above.

### Experimental Section

### Materials and Methods

### Chemical Reagents

All solutions were prepared with Type I water. Hexadecyltrimethylammonium bromide (CTAB, >99%), hexadecyltrimethylammonium chloride (CTAC, >99%) and hydroquinone (99.5%) were purchased from Acros Organics. Gold (III) chloride trihydrate (HAuCl₄, 99.9%), ascorbic acid (99.9%) and silver nitrate (AgNO₃, 99.9999%) were purchased from Sigma-Aldrich. Hydrochloric acid (37%) and sodium hydroxide (98%-100.5%) were purchased from Honeywell Fluk. Orio pH buffer solutions (4.0, 7.0 and 10.0) were purchased from Thermo Scientifi. All the reagents were used as received. The procedure to prepare the stock solutions is discussed below.

### Platform

The platform was constructed in house from a range of 3D printed, laser-cut and commercially available components. A full bill of materials and assembly instructions is described below. The software control of the platform for basic operations was written in Python 3.

### The Extinction Spectrum Simulation Engine (PyDScat-GPU)

The software was used for GPU-accelerated extinction spectrum simulation of metallic nanoparticles based on the discrete-dipole-approximation (DDA) method and written in Python 3 using Tensorflow 2.0. The full details are available in Section 2 below.

### Algorithms and Data Analysis

The details of benchmarking the algorithms in the simulated chemical spaces are described in Section 2 below, where the codes were written in Mathematica 12.0. The details of the spectral data analysis the algorithms in the experimental exploration and optimisation of the three chemical spaces are available in Sections 3 and 4 below. Extra TEM images for the nanoparticles discussed during exploration and optimisation are discussed below. The codes for the experimental implementation of both algorithms were written in Python 3 (https://github.com/croningp/NanomatDiscovery) and connected to the control software of the platform to establish a closed loop.

### Directed Graph Structure and Hash Representation

The code to generated directed graphs was written in Python 3 using NetworkX. The control software can read the directed graph and execute the operations defined in the graph. The digital signatures of the nanoparticles were generated from the string format of the chemical synthetic procedures written in χDL. Full details are available described below and https://github.com/croningp/NanomatDiscovery.

### 1. Platform Hardware

### 1.1 Overview

The overall platform architecture of the Autonomous Intelligent Exploration, DIScovery and Optimisation of Nanomaterials (AI-EDISON) system consists of three main assemblies: a central chemical reaction module (CRM), a series of high accuracy syringe pumps and a flow spectroscopic suite. The CRM was an upgraded version of liquid handling platform called the Modular Wheel Platform (MWP)¹ with advanced capabilities required for this work. This custom reaction platform was constructed using a combination of 3D printed, laser cut and commercially available components, capable of reaction dispensing, stirring, pH control and sample extraction for analysis/storage. This advanced MWP and analysis hardware allowed a closed-loop system to be created for the algorithm driven exploration and optimisation of gold nanoparticles (Au NPs). As a basic version of the MWP has been reported previously in detail, it will only be briefly discussed here.

The system was controlled using a Lenovo ThinkCentre (Intel i5, 8 GB RAM) running on Linux/Ubuntu. Custom hardware responsible for liquid handling/performing reactions was controlled by in-house Commanduino software library (https://github.com/croningp/commanduino) via RAMPS v1.4 shield connected to Arduino MEGA 2560 prototype board. Syringe pumps (Tricontinent Ltd, C-3000 series) used for liquid handling operations were controlled using an in-house developed python library (https://github.com/croningp/pycont). All software and modules to control the complete platform were written in Python 3. A high-performance QE-PRO spectrometer was used in this system in combination with a PEEK FIA-Z-SMA 905 flow cell (10 mm path length) and DH-2000-S light source, all from Ocean Insight Ltd. The system is also equipped with a NIR-Quest IR high performance spectrometer, and a second QE-PRO configured for Raman. Control of this spectrometer was achieved via the SeaBreeze python library from Ocean Insight Ltd. pH measurement was carried out using a standard VWR semi-micro probe pH electrode and data logger (DrDAQ, Pico Technology Ltd) was used for data acquisition. The procedure to control pH of reaction solutions was detailed in Section 1.3. 1/16″ and ⅛″ PTFE tubing from Cole Palmer was used in combination with PFA flangeless fittings Luer connections for all liquid transfer. Structural hardware for the platform is built using commercially available OpenBuilds profile and fixtures as well as laser cut acrylic sheets. For moving parts and general custom hardware, a range of commercially available components were used in combination with 3D printed parts developed as part of the platform's modular architecture. All 3D printed components were designed using Onshape (https://www.onshape.com/en/), a cloud-based CAD software and printed using an Objet500 Connex from Stratasys in standards FullCure720 RGD material. Laser cutting of 4- and 6-mm acrylic was performed on a Monster laser ML 1060 with a 130W CO2 laser from Radecal Machines. All design (STL, DXF) and construction files can be found at https://github.com/croningp/NanomatDiscovery.

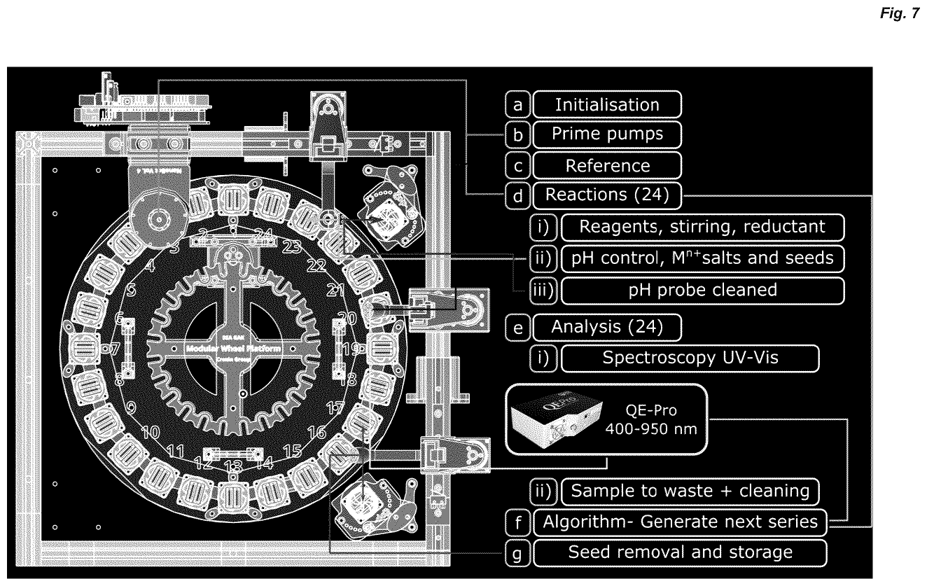

A flow diagram showing the platform capabilities during a 24-reaction batch can be seen in FIG. 7. Detailed description of each hardware module's concept and design are included in this section in the order they appear in this flow diagram.

### 1.2 the Chemical Reaction Module and Pump Control

Reactions are performed on the chemical reaction module. In short, the platform uses a Geneva drive to turn a tray of 24 reactions vessels, each stirred from below using a magnetic stirring mechanism. Pumps provide reaction materials at any desired position of the wheel via several 3D printed dispensing units. Multiple modules can be attached to the v-slot profile frame of the platform to access any position around this reaction tray. The functions of these modules can range from: dispensing, probe analysis, cleaning, etc., all of which are custom designed for the system and can be added and removed with ease in terms of both hardware and control software. For the chemical reaction used here: dispensing, pH measurement and control, probe cleaning, sample transfer for analysis and vial/flow cells cleaning were required to complete a reaction sequence, which typically consisted of 24 reactions. Each module for the functions listed above will be detailed individually.

A series of four- and six-valve C3000 series syringe pumps from TriContinent were used with syringe volumes ranging from 100 μL to 5 mL as requested. The pumps are connected to TriCont hubs and are controlled directly with our in-house PyCont software library.

Up to 15 pumps can be powered and controlled via a single custom designed hub (TriCont hub), created in house. TriContinent C3000 pumps use a standard DA-15 connector for both data and power. They implement three different protocols for communication-RS-232, RS-485 and CAN-signals for which are available in the output connector. To make the whole pump assembly and the cabling more compact and avoid manual daisy-chain cable crimping, a simple hub unit was made. The hub consisted of a PCB sandwiched between two sheets of acrylic (4 mm thick for the front; 6 mm thick for the back) acting as a case. The acrylic sheets were fixed to the board by means of standard PCB standoffs. The PCB itself carried 15 standard straight DA-15 female connectors in a 5×3 matrix. This number is governed by the address limitation of the pumps themselves—the address is set with the rotary switch at the back which has 15 possible positions. Multiple hubs could be used for more than 15 pumps. The RS-485 A and B signals from all connectors were connected in series and length-matched. No termination was implemented as the pumps had embedded switchable line terminator. The power pins in the connectors were connected to the top and bottom plane of the PCB which carry VCC and GND polygons. The top acrylic sheet had cut openings for the DA-15 connectors. The pumps were connected to the board using standard DA-15 female-to-female cables. The board was connected to the computer by means of USB to UART converter cable from FTDI Ltd.

### 1.3 Performing Reactions, pH Measurement and Control

The order of addition of reagents in nanoparticle synthesis is crucial to produce high quality species. In a typical seed mediated synthesis of the type used throughout this work (e.g., synthesis of Au nanorods), HAuCl₄ is reduced in the presence of a concentrated surfactant solution, using a weak reducing agent, in our case ascorbic acid or hydroquinone. Next a symmetry breaking agent may be added e.g., AgNO₃ and finally, a pre-synthesised Au seed is added. In our system we wanted to accurately control the pH of the growth solution in which the particle forms. To avoid the direct reduction of metallic species on the pH probe, the measurement and control of the pH was performed before the addition of any metallic species. To do this, we move the current working vial, four positions clock-wisely from the dispensing position to be accessed by a probe. The probe is mounted on a module capable of horizontal and vertical motion, reaching two pre-set positions: reaction vial position which is four positions away from the dispensing position, and a wash station positioned alongside the vial tray. The module comprises two Nema11 lead screw motors to achieve the X and Z motion along the platforms frame. 3D prints bind the lead screws of these motors to precision steel rails and carriages that provide smooth motion. Mechanical endstop switches define the home positions of these motors. Attached to the Z motion, the 3D print is a holder for the pH probe as well of tube guides for the acid and base solutions used to modulate the pH. Details of the strategy developed to reach the desired pH as efficiently as possible can be found in Section 3.3. Once a reaction has reached the target pH, the module returns to the cleaning vial containing Type I ultrapure water as a cleaning solution. The vial is stirred from bottom using the same mechanism as the main vial tray. This vial is cleaned, and the solution replenished in parallel to the operations of the wheel to prevent contamination between samples and also acts as a storage position of the pH probe once the sequence is finished by dispensing saturated KCl (3 M). The pH probe is calibrated autonomously each day by performing a similar series of actions between three buffer solutions on the vial try and the wash station.

### 1.4 Sample Analysis

Once a pre-defined growth period has passed, the samples are analysed autonomously by UV-Vis spectroscopy. A modular syringe driver (MSD) is secured to the platform frame at vial position seven (position one being the dispensing position with position index increased clock-wisely), with a 3D printed multichannel tube attachment. This unit houses tubes which lead to different locations like the UV-Vis flow cells or are used for stock solutions in flow cell wash cycles. For sample extraction the unit is lowered into the reaction solution and a dedicated pump moves 5.0 mL of material through the UV-Vis line first. Then the pump drives another 5.0 mL of the sample through the flow cell at high speed to prevent bubbles remaining in the sample lines. The volume was varied to 3.5 mL in chemical space 2 considering the possible smallest volume of the sample.

The outlet of the flow cell is directed back into the multichannel attachment to return the sample to its vial. The UV-Vis spectra are recorded using a high-performance QE-PRO absorbance spectrometer from Ocean Insight (400-950 nm). The sample is then removed to waste from the same position and the vial filled with Type I ultra-pure water to be flowed through the sample path multiple times. This wash cycle repeats five times. The MSD is removed from the vial and the next sample moves to the analysis position. This cycle is repeated for all samples in a step of 24 experiments.

### 1.5 Seed Transfer and Storage

In performing the multistep synthesis, many of the samples produced are both products and future reagents as the seeds. For each nanoparticle, several repeats of the sample can be performed on the wheel (More details in Section 5). One or more of these samples needed to be transferred to an off-wheel location for later use. For this, a module that is a combination of the sample extraction and pH modules was built. It requires the same X and Z motion of the pH assembly and a similar 3D printed multichannel part to accommodate extraction and cleaning tube. Before the addition of seed, the seed transfer unit would move to a vial position, remove a portion of the sample and store the sample in a pump temporarily. By turning the wheel and moving the unit back to the vial position, the stored solution can be used as seed. Once this seed solution was used, the tube in the module and the pump connecting to it will be cleaned in a wash station.

### 1.6 Temperature Control

The entire liquid handling system was contained in a temperature-controlled box set to 30° C. There are many reasons for this include preventing surfactants in stock or reaction solutions from precipitation, keeping the growth temperatures identical throughout the discovery process and for the reproducibility of samples. The box itself was a simple structure of v-slot aluminium rails with 4 mm acrylic sheets as boundaries. The temperature inside the platform box was controlled using an RE72 PID temperature controller from Lumel (configuration with T-type thermocouple input). The PID settings were determined upon first start-up using the in-built autotune function (Ziegler-Nichols method). T-type thermocouples were used as sensors as they have better accuracy compared to more commonly used K-type thermocouples. To provide an average reading over the whole box, five individual thermocouples were connected in parallel and secured evenly around the interior of the box. All the thermocouples were two meters long, however an extra swamping resistor was connected in series with each thermocouple to compensate for possible difference in resistance. The on/off output of the PID controller was fed into a Crydom D2410 solid-state relay controlling the fan heater. The fan heater was mounted on the ceiling with the air flow directed to the side wall to cause minimal disturbance inside the box. The PID controller and all accompanying electronics were in a small acrylic box mounted outside the heated volume. The connections for the fan heater and the thermocouples were fed into the main box through the brush plate pass-through. The system was started at least one hour before the actual experiment started to allow the temperature inside the box to become steady.

### 2. In Silico Exploration of Au and Au—Ag Bimetallic Nanoparticles

The exploration and optimisation algorithms based on Multi-dimensional Archive of Phenotypic Elites (MAP-Elites) and Global Search with Local Sparseness (GS-LS) to search nanoparticles were benchmarked in simulated chemical spaces. In Section 2.1, we will give a brief introduction to both algorithms. Section 2.2 will describe a general discrete dipole approximation (DDA) method to simulate the extinction spectra of arbitrarily shaped metallic nanoparticles. Based on it, a GPU-accelerated Python (PyDScat-GPU) package was developed utilising Tensorflow 2.0 for efficient scattering simulations. In Section 2.3, a family of different shapes were generated with superellipsoid as the shape descriptor. Using these shapes as templates, the same shaped Au and Au—Ag bimetallic nanoparticles were created in silico and the corresponding extinction spectra were simulated with DDA. In Section 2.4, the Au and Au-Ag bimetallic nanoparticles and their spectra from Section 2.3 were used to create two simulated chemical spaces respectively. In Section 2.5, the two simulated chemical spaces were explored with the exploration algorithm, with its performance benchmarked to Random Search, where the whole space is sampled by a uniform distribution. With all the data available from the exploration, we further benchmarked the optimisation algorithm to tune the sophisticated UV-Vis features in the Au—Ag bimetallic space in Section 2.6.

### 2.1 Algorithms

### 2.1.1 Definitions

Considering a N-dimensional input chemical space that needs to be searched, any sampling point in the space is represented by a vector x={x₁, x₂, . . . , x_{N}}, where xᵢ is the input value in the i^{th} dimension. The input set of existing samples is defined as X={x₁, x₂, x₃, . . . , x_{M}}, where xⱼ is a vector representing the input of j^{th} sample. The spectra of the samples are available by sampling the space, which are defined as Y={y₁, y₂, y₃, . . . , y_{M}}, where yⱼ represents the spectrum of the j^{th} sample.

The behaviour of the sample can be estimated by their attributes (a(y)) based on the spectral observation, which includes the number of the UV-Vis peaks and the positions. The set of the samples' attributes is denoted as A={a₁, a₂, a₃, . . . , a_{M}}. The attributes were used for classification, and the set including the classes is denoted as C={c₁, c₂, c₃, . . . , c_{M}}, where cⱼ is the class index of the j^{th} sample. Based on the spectra, the performance of the sample can be quantified by a fitness function (F(y)). This fitness function is correlated with the desired UV-Vis features during the search. Thus, a set of fitness values is defined as F={F(y₁), F(y₂), F(y₃), . . . , F(y_{M})}, where F(yⱼ) represents the fitness value of the j^{th} sample. The fitness function (F) can be defined either dependent or independent of the class index, which will be discussed when the algorithms were implemented below. The sample with the highest fitness (performance) within one class was defined as an elite, which guides the exploration process later.

### 2.1.2 Exploration Algorithm Based on MAP-Elites

Inspired by the literature of evolutionary algorithm, MAP-Elites² is an illuminating search algorithm designed to explore a feature space. The feature space was defined by both the performance and the behaviour of the samples. The procedure was as following:
1. For samples (X), obtain their spectra (Y) by sampling the space.
2. Based on the spectra, obtain the samples' attributes (A).
3. Classify the samples according to their attributes (A) to obtain the corresponding class set (C).
4. Calculate the fitness (F) of the samples according to their spectra (Y) and classes (C).
5. Select the sample with the highest fitness within one class as an elite. Get a set of elites (E) from different classes.
6. Use the elites as the parents to crossover and mutate their input variables, which will generate a set of new samples. A small portion of random sampling can be added.

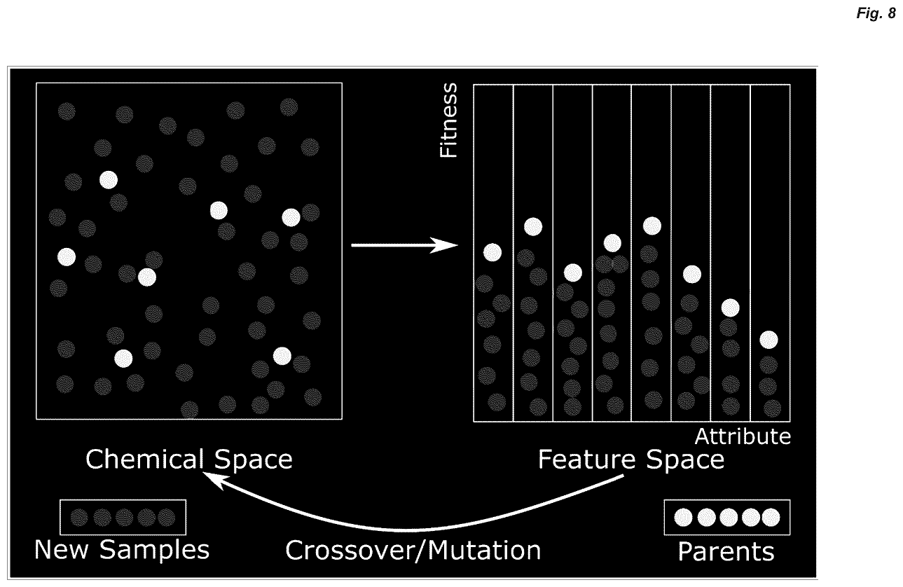

The above procedure defines one step of exploration. It iterated multiple steps until the exploration was complete, see FIG. 8.

### 2.1.3 Optimisation Algorithm Based on Global Search with Local Sparseness (GS-LS)

The optimisation algorithm was based on global search with local sparseness (GS-LS), which was inspired by the novelty-search algorithm³ that considers the novelty of a sample by measuring its local sparseness in a behaviour space. However, in our system we have focused this measure of local sparseness in the input space instead. Local sparseness in the input space is defined as the local sampling density around the current sample of interest. The algorithm then considers the local spareness of data points around a given sample as part of the overall fitness measure. We then use the lack of or abundance of samples in particular regions to encourage the search in less sampled regions for a global search.

In the optimisation, a desired UV-Vis spectrum was set as the target. The aim is to find multiple conditions that locally have the most similar UV-Vis spectra to the target. By considering the local sparseness in the input space and adding it to the fitness function, the algorithm was encouraged to search less-sampled regions, which helps to escape from local maxima and also to search for samples that are separated in the input space but still show high fitness. The local sparseness(S) near a sampling point (x) in the input space was measured by the average distance from its K-nearest neighbours (x′ᵢ) as shown in Eq. (1). A similarity metric (M_{S}) considering the difference of peak positions and the whole spectrum between the sample and the target was defined to guide the optimisation (Eq. (2)). A linear summation of both local sparseness (S, Eq. (1)) and similarity between the target and the sample (M_{S}, Eq. (2)) was used to define the fitness (F, Eq. (3)).

$$S ( x ) = \frac{1}{K} {\sum }_{i = 1}^{K} dist ( x , {x}_{i}^{′} ) \qquad ( 1 )$$

where dist(x, x′ᵢ) measures the Euclidean distance between x and x′ᵢ in the input space, and x′ᵢ is the i^{th} closest neighbour to x.

$${M}_{S} = - \vert p - {p}_{target} \vert - {k}_{1} {\sum }_{i} \vert {I}_{x , i} - {I}_{target , i} \vert + {k}_{2} \qquad ( 2 )$$

where p and p_{target} are the peak positions of the highest peak in the UV-Vis spectra of the sample and the target. I_{x,i} and I_{target,i} are the i^{th} intensity of the UV-Vis data of the sample and the target respectively. k₁ is used to tune the importance between constraining the peak position and increasing the overall similarity of the spectra. When two identical UV-Vis spectra are found, both |p−p_{target}| and Σᵢ|_{x,i}−I_{target,i}| reduces to 0 and k₂ puts an upper boundary of the similarity metric.

$$F = {M}_{S} + {k}_{3} S \qquad ( 3 )$$

where k₃ is a coefficient to tune the relatively importance between M_{S} and k₃. Then an optimiser can be used to increase the fitness. We used the evolutionary algorithm (EA) as the optimiser in this paper.

After the optimisation, all the samples' UV-Vis similarity to the target were quantified. Multiple solutions were selected so that for every solution, compared to its K-nearest neighbours, it has the most similar UV-Vis to the target according to similarity metric (M_{S}). They can represent the local optimal solutions in the observation set. The values of K in calculating the local sparseness and selecting the solutions are not necessarily the same. Depending on the target UV-Vis and chemical space, these solutions can be close or distinct in the input space, which can correspond to nanoparticles of similar or completely different shapes.

### 2.2 Discrete Dipole Approximation

The electromagnetic properties of metallic nanoparticles are closely correlated to their structures due to the plasmon resonance effect. Theoretical tools to study the electromagnetic field of arbitrarily shaped nanoparticles include the finite difference time-domain (FDTD)⁴, the boundary element method (BEM)⁵ and the discrete dipole approximation (DDA)⁶. Here we used the DDA to simulate the extinction spectra of nanoparticles. The DDA, with a publicly available software DDSCAT developed by B. T. Draine and P. J. Flatau⁶, is widely used to study the optical properties of nanostructures. In this method, the nanoparticle was discretized into N point dipoles as an approximation. Every dipole is induced by the incident beam as well as the electric field from other dipoles. The system composed of dipoles is self-consistent and can be solved. It requires solving a linear system with 3N equations. In DDSCAT, the complex-conjugate gradient (CCG) and fast Fourier-transform (FFT) method is used to solve the linear system where the time cost is O(N³). With the development of graphics processing unit (GPUs), the operations on the matrix are faster and we can alternatively solve the linear system by directly matrix inverse. Taking the advantages of GPUs, we developed a Python package, PyDScat-GPU, to simulate the UV-Vis spectrum efficiently.

### 2.2.1 Methods

First, the nanostructure was discretized into N polarizable cubic lattices which represent the point dipoles. Every point dipole's polarizability was associated with the local dielectric constant. The dipole was induced by the incident beam and also the electric field from the rest of the dipoles. To solve the dipoles and make them self-consistent, the system can be described by the simplifying the Maxwell equations into a set of linear equations (Eq. (4)).

$$A P = E \qquad ( 4 )$$

where E is a 3N vector describing the local electric field of the incident wave in every dipole position, A is a 3N by 3N matrix depending on the geometry and materials of the nanoparticle and P is a 3N vector describing the solutions for the individual dipoles. See reference⁶ for full details of this linear system.

After solving P, we can further evaluate the local electric field distribution, extinction and absorption cross-sections via the following Eq. (5), Eq. (7), and Eq. (8) respectively.

$${E}_{i} = {E}_{i , inc} + {\sum }_{j}^{N} {E}_{j} \qquad ( 5 )$$

where Eᵢ denotes the electric field at position rᵢ, E_{i,inc} is the electric field from the incident beam and Eⱼ is the contribution from the j^{th} dipole located at rⱼ and can be formulated as:

$${E}_{j} = - \frac{{e}^{{ikr}_{i , j}}}{{r}_{i , j}} \{ {k}^{2} {r}_{i , j} \times ( {r}_{i , j} \times {P}_{j} ) + ( \frac{1 - {ikr}_{i , j}}{{r}_{i , j}^{2}} ) \times [ {r}_{i , j}^{2} {P}_{j} - 3 {r}_{i , j} ( {r}_{i , j} \cdot {P}_{j} ) ] \} \qquad ( 6 )$$

where r_{i,j}=rᵢ−rⱼ, k is the wavenumber and Pⱼ is the j^{th} solved dipole.

$${C}_{ext} = \frac{4 π k}{{\vert {E}_{inc} \vert}^{2}} {\sum }_{j}^{N} Im ( {E}_{inc , j}^{*} \cdot {P}_{j} ) \qquad ( 7 )$$

where Cₑₓₜ is the extinction cross-section, Im(x) denotes the imaginary part of x and x* is the conjugate of x.

$${C}_{a bs} = \frac{4 π k}{{\vert {E}_{inc} \vert}^{2}} {\sum }_{j}^{N} \{ Im [ {P}_{j} \cdot ( {α}_{j}^{- 1} ) * {P}_{j}^{*} ] - \frac{2}{3} {k}^{3} {P}_{j} \cdot {P}_{j}^{*} \} \qquad ( 8 )$$

where C_{abs} is the absorption cross-section and αⱼ is the polarizability of the j^{th} dipole. In this paper, we use the “filtered coupled dipole” (FCD) method⁷ to calculate the polarizability (Eq. (9)-(11)).

$${α}_{j} = \frac{{α}_{j , CM}}{1 + D} \qquad ( 9 )$$

where α_{j,CM} is the Clausius-Mossotti polarizability⁸ defined below:

$${α}_{j , CM} = \frac{3 {d}^{3}}{4 π} \frac{( {m}_{j}^{2} - 1 )}{( {m}_{j}^{2} + 2 )} \qquad ( 10 )$$

where mⱼ is the complex refractive index of the j^{th} dipole and d is the dipole length. The D term in Eq. (9) is defined as:

$$D = \frac{{α}_{j , CM}}{{d}^{3}} [ \frac{4}{3} {( kd )}^{2} + \frac{2}{3 π} ln ( \frac{π - kd}{π + kd} ) {( kd )}^{3} + \frac{2}{3} {i ( kd )}^{3} ] \qquad ( 11 )$$

where k is the wavenumber of the incident beam.

When considering multiple-metallic nanostructures, the polarizability of the dipoles should be modified accordingly. In PyDScat-GPU, the refractive indexes of individual dipoles were estimated and further used to calculate the polarizability via FCD method. By default, a simple and empirical relation was used to determine the refractive index depending on the components and their portions:

$${m}_{j} = {\sum }_{k} {w}_{j , k} {m}_{j , k} \qquad ( 12 )$$

where w_{j,k} is the portion of component k in the j^{th} dipole. The polarizability can also be defined by the user, which might be from experimental measurement.

Once Cₑₓₜ and C_{abs} are available, the extinction and absorption efficiency factor can be transformed from the corresponding cross-sections via Eq. (13).

$${Q}_{ext / a bs} = \frac{{C}_{ext / a bs}}{π {r}_{eff}^{2}} \qquad ( 13 )$$

where r_{eff} is the effective radius of a sphere with the same volume as the nanoparticle.

The extinction efficiency factors from several orientations of the simulated nanoparticle are calculated and averaged in the simulation to give the final UV-Vis spectrum. To enable the accuracy, the validation criteria⁶ (Eq. (14)) is always satisfied in our calculations.

$$\vert m \vert kd < 0.5 \qquad ( 14 )$$

where m is the complex dielectric constant of the material, k is the wavenumber of the incident beam and d is the dipole length.

### 2.2.2 Results

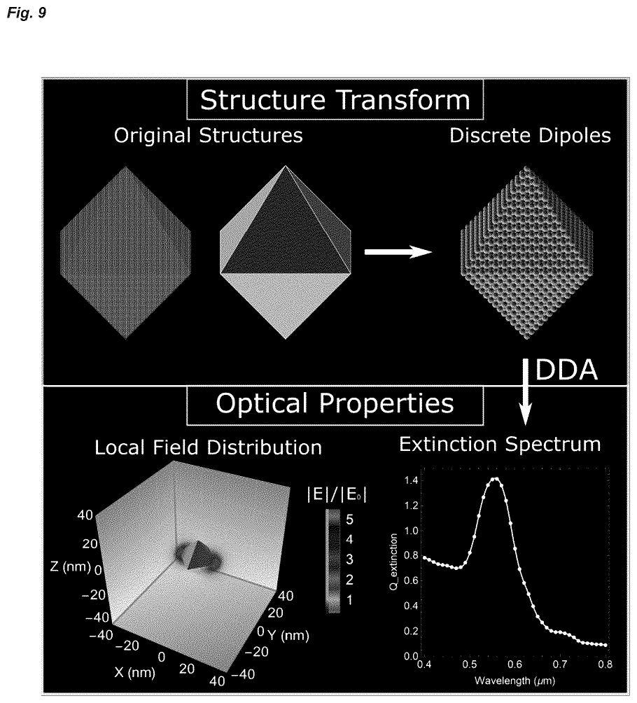

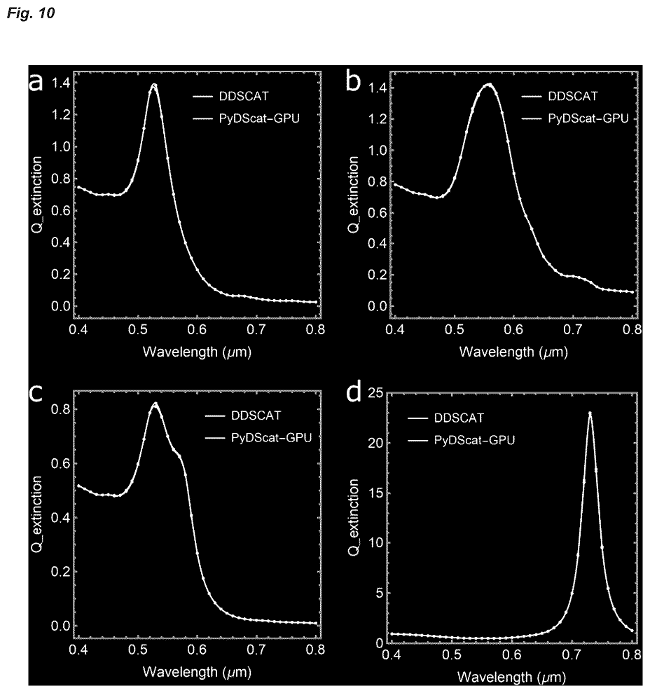

An example of simulating the optical properties using PyDSCAT-GPU for Au octahedron (edge length ≈20 nm) can be seen in FIG. 9. Either an atomic model or a continuum geometry can be initially transformed to a set of dipoles for further calculations. To validate our package, we further compared the extinction efficiency factors from it with those from DDSCAT on the same Au nanoparticles including spheres (radius ≈10 nm), octahedra (edge length≈20 nm) and both transverse and longitudinal modes of rods (10×10×30 nm cuboid). They all showed consistent results with neglectable difference (FIG. 10).

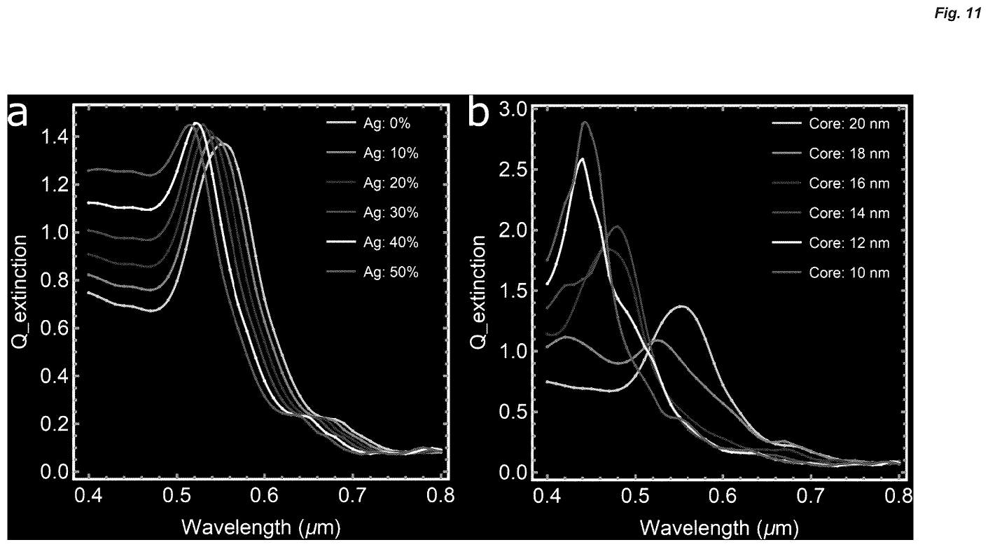

Regarding the multiple-metallic nanostructures, another two examples are shown in FIG. 11. In both examples, a bimetallic octahedron (edge length ≈20 nm) consisting of Au and Ag atoms was created.

In the first example, it is assumed the doping of Ag in the Au nanoparticle was uniform so that all the dipoles had the same portions of Au and Ag, thus sharing the same refractive index and polarizability. In the simulation, the peak blue-shifted with an increased portion of Ag (FIG. 11a).

In the second example, we simulated the UV-Vis spectra for a series of Au@Ag core-shell structures, where the dipoles belonging to the core were composed purely of Au and the shell purely of Ag. The shape of the nanoparticle was an octahedron with an edge length of 20 nm. The core was set to be an octahedron with its edge length decreased gradually from 20 nm to 10 nm, and the simulation showed the emergence of the Ag feature peaks between 400 nm and 500 nm due to the expansion of the Ag shell (FIG. 11b).

### 2.3 Au/Au—Ag Bimetallic Nanoparticles Using Superellipsoid as the Shape Descriptor and their Spectroscopic Properties

Before creating the simulated chemical space, we created a geometry set consisting of a variety of shapes originating from the superellipsoid, which can be defined by Eq. (15).

$${( {\vert \frac{x}{a} \vert}^{r} + {\vert \frac{y}{b} \vert}^{r} )}^{t / r} + {\vert \frac{z}{c} \vert}^{t} \leq 1 \qquad ( 15 )$$

where (a, b, c) and (r, t) tunes size and the curvature of the superellipsoid respectively.

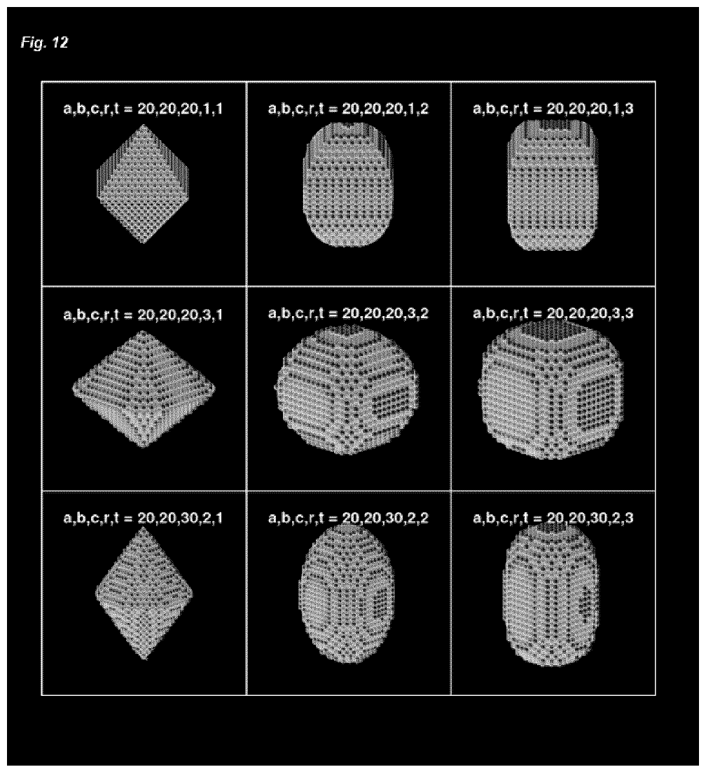

In our simulation, we set a=b=20 nm and varied c in the range of [20, 60] nm with a constant interval of 5 nm to introduce anisotropy. The curvature of the shape was tuned by changing both r and t in the range of [1,3] with an interval of 0.2. By changing the parameter set (c, r, t) and keep a=b=20 nm in Eq. (15), we created a 9×11×11 geometry set including spheres, octahedra, rods, bipyramids, etc., (See FIG. 12 for examples). The dipoles were all approximated from the continuum geometries. Then the corresponding extinction spectra of nanoparticles with the same shapes can be simulated with PyDScat-GPU as described in Section 2.2. Note geometrical error in representing the original shape with dipoles could be introduced when converting a continuum geometry into the dipoles.

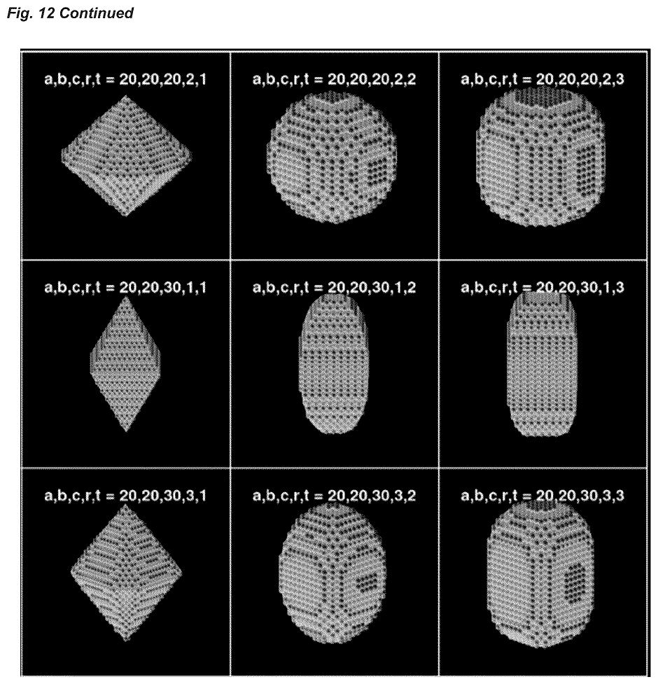

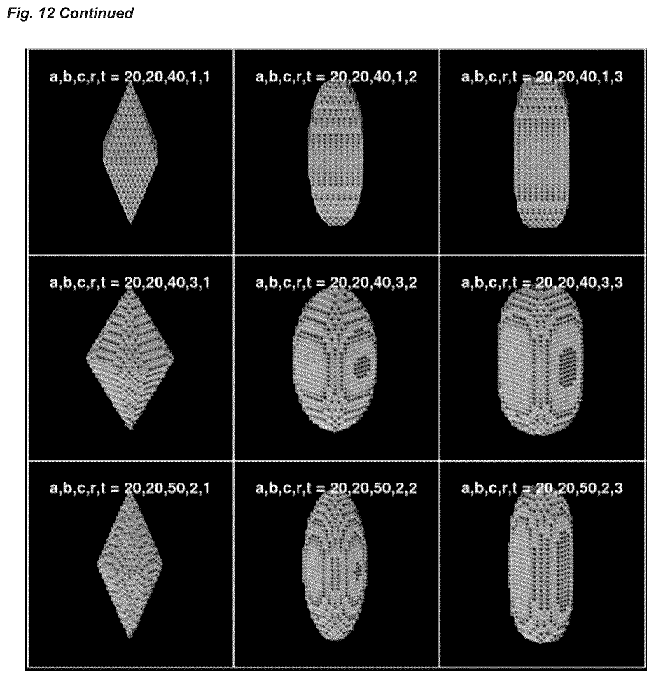

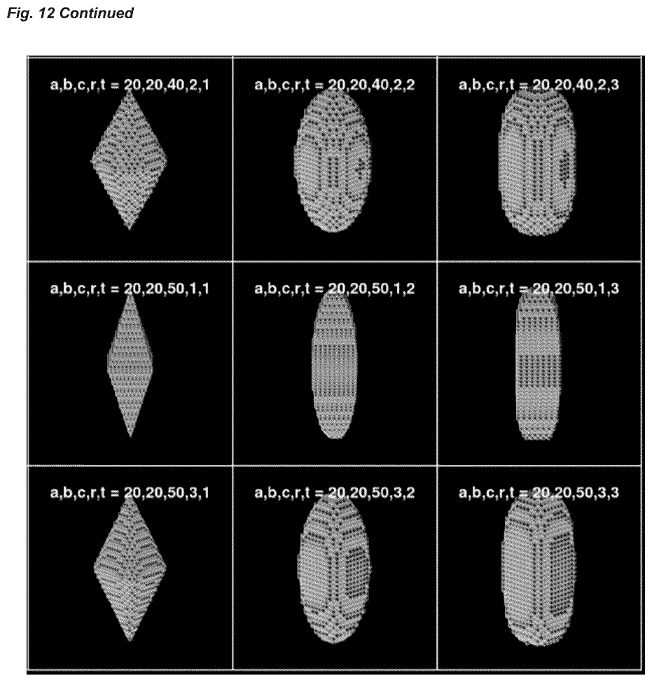

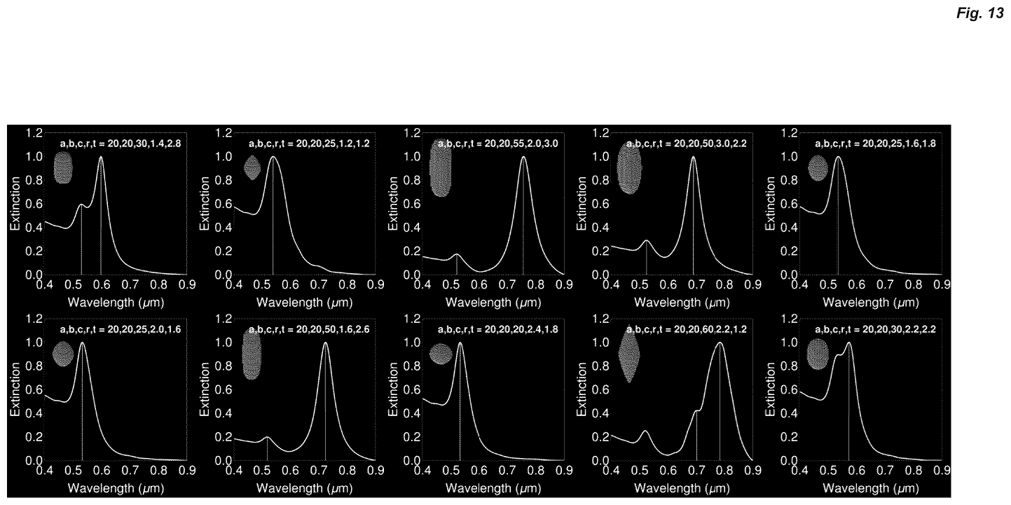

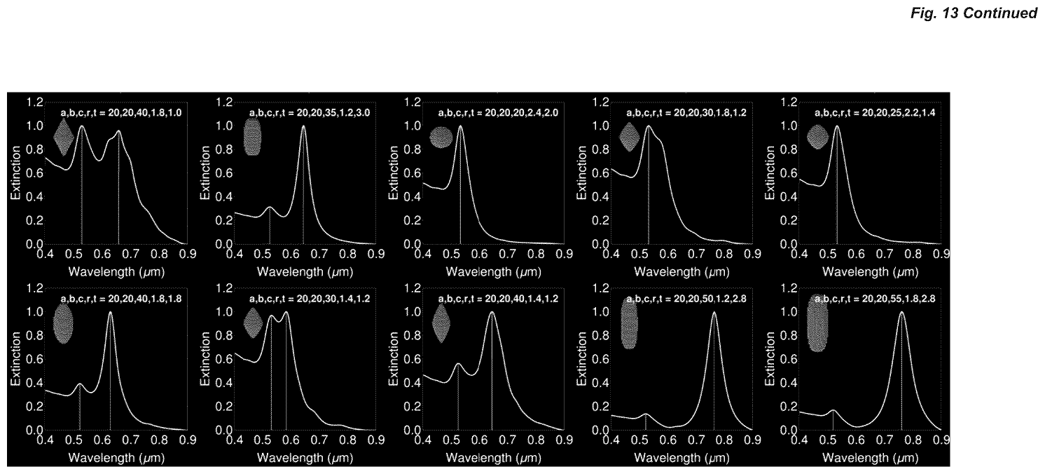

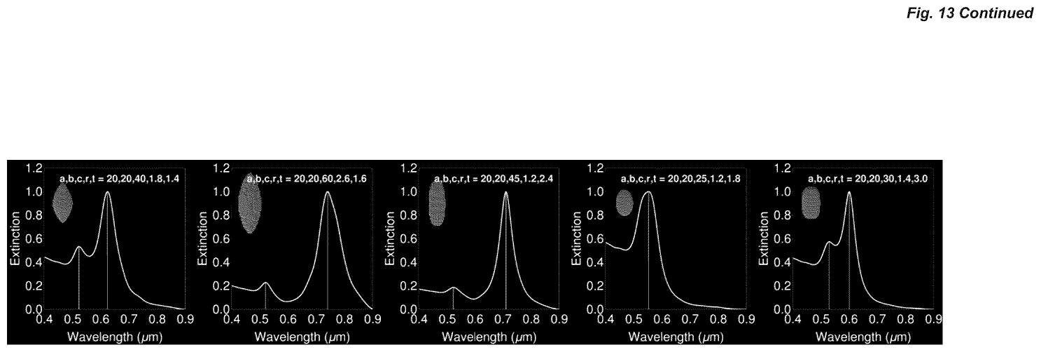

For the first simulated chemical space, the dipole component is purely Au. Thus, a 9×11×11 set of Au nanoparticles originated from the geometry set can be created and will be used to define the first simulated chemical space (See FIG. 13 for examples and their corresponding UV-Vis spectra).

For the second simulated chemical space, the dipole is composed of both Au and Ag, where the portions of one dipole depend on a dipole component function (DCF). Thus, a set of nanoparticles with not only different shapes but also compositions can be created. For a single dipole in the nanoparticle with its geometry defined by the parameter set (a, b, c, r, t), we first calculate its “relative distance” d_{R} via Eq. (16), which indicates the relative position of the dipole to the centre:

$${d}_{R} = {( {\vert \frac{x}{a} \vert}^{r} + {\vert \frac{y}{b} \vert}^{r} )}^{t / r} + {\vert \frac{z}{c} \vert}^{t} \qquad ( 16 )$$

where (x, y, z) is the dipole position.

The Ag and Ag portions in the dipole are determined by the relative distance (d_{R}) through Eq. (17) and (18).

$${p}_{A g} = \frac{1}{1 + {e}^{- {v}_{DCF , 1} ( {d}_{R} - {v}_{DCF , 2} )}} \qquad ( 17 )$$

$${p}_{A u} = 1 - {p}_{A g} \qquad ( 18 )$$

where v_{DCF,1} and v_{DCF,2} are changeable coefficients. These equations defined a higher portion of Ag in the outer layer of nanostructures.

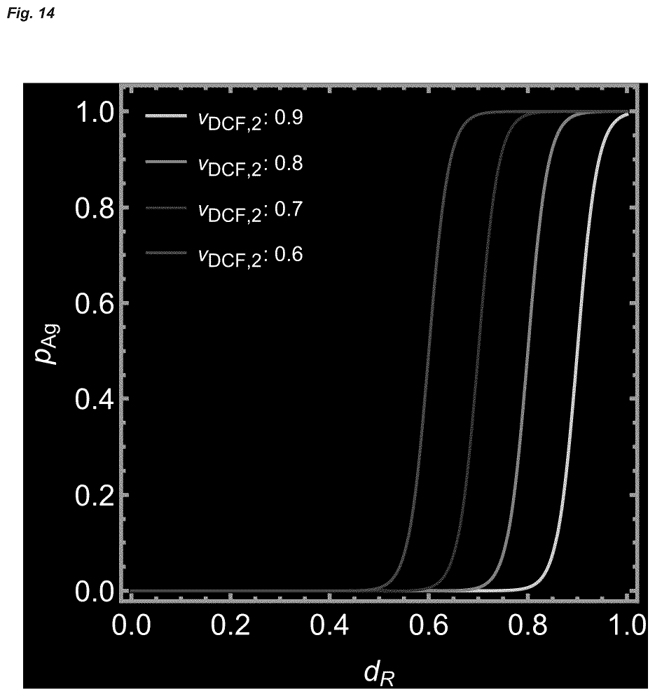

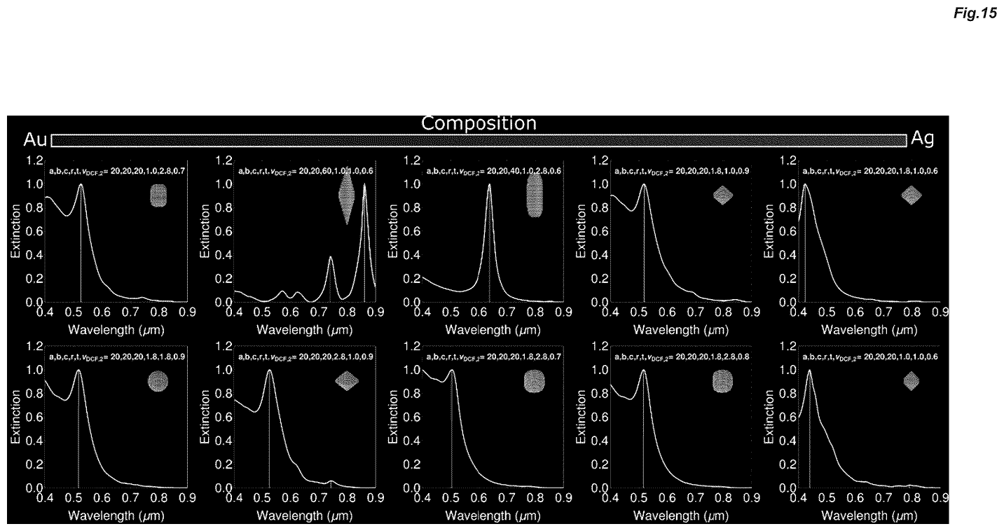

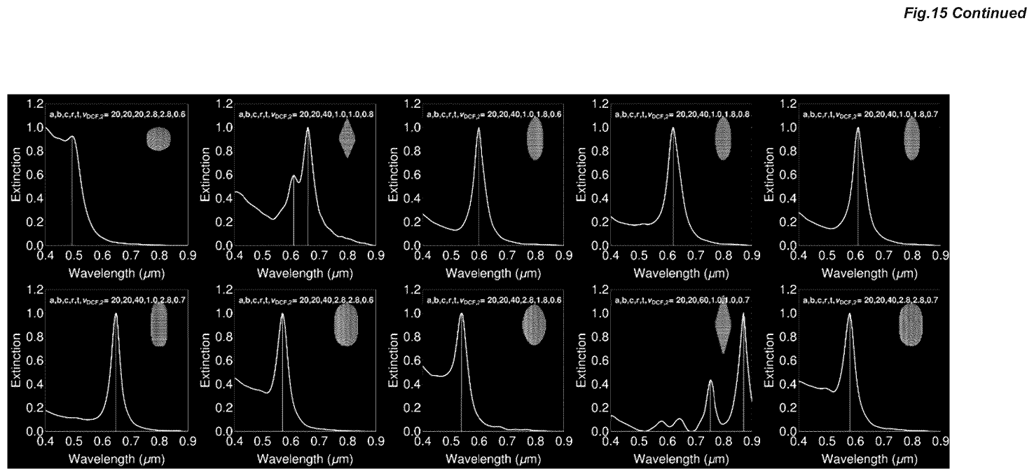

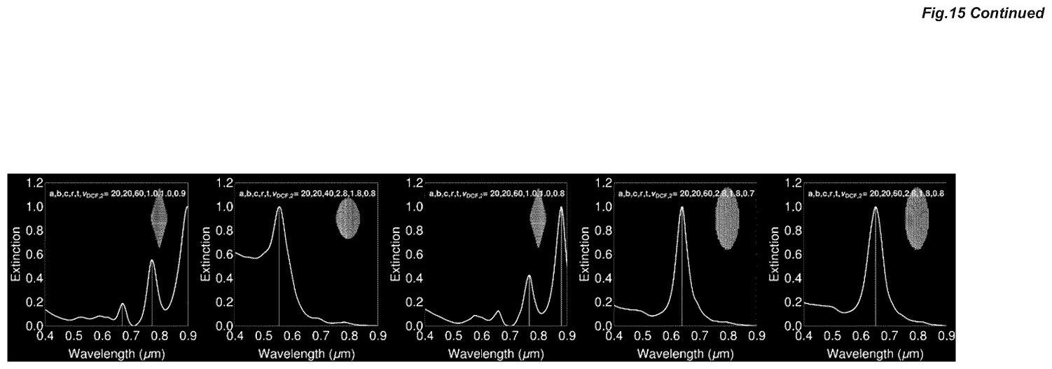

By keeping v_{DCF,1} as 50 and varying v_{DCF,2} from 0.9 to 0.6 with an interval of 0.1, the distribution of Ag in the out layer in the nanostructures was changed (FIG. 14). Thus, a 9×11×11×4 set of Au—Ag bimetallic nanostructures was created with all the shapes originated from the geometry set but distinct v_{DCF,2}. Their extinction spectra were further simulated to create the second simulated chemical space, which will be discussed below (See FIG. 15 for examples and their corresponding UV-Vis spectra).

### 2.4 Simulated Chemical Space from Uniquely-Shaped Nanoparticles

### 2.4.1 Simulated Chemical Space 1

The first chemical space was created utilising the 9×11×11 Au nanoparticle set, which is generated by varying the superellipsoid parameters (c, r, t) as described in Section 2.3. The input variables in the chemical space include three variables (v₁, v₂, v₃) that can change the values of (c, r, t), thus varying the shapes of the nanoparticles. A fourth variable v₄ was used to introduce the Au octahedra as by-products. All the variables of (v₁, v₂, v₃, v₄) are in the ranges from 0 to 1.

Since the Au nanoparticle set is discrete and originates from different geometries with values of (c, r, t), a linear transformation and a piece-wise rounding function was used to map the first three input variables (v₁, v₂, v₃) to the geometry parameters (c, r, t) as following: First, each of the (c, r, t) variables was sorted in ascending order. For the sampling point in the input space with (v₁, v₂, v₃), we need to determine which (c, r, t) values this point corresponds to. The i^{th} value of c, j^{th} value of r and kth value of t were chosen according to the values of (v₁, v₂, v₃), with the indexes being determined by Eq. (19), (20) and (21) respectively.

$$i = 1 + ⌈ 8 {v}_{1} ⌋ \qquad ( 19 )$$

$$j = 1 + ⌈ 10 {v}_{2} ⌋ \qquad ( 20 )$$

$$k = 1 + ⌈ 10 {v}_{3} ⌋ \qquad ( 21 )$$

where ┌x┘ outputs the nearest integer to x.

For a given point (v₁, v₂, v₃, v₄), the sample is composed of (1−v₄) amount of the Au nanoparticles with their geometry determined by (v₁, v₂, v₃) as described above, together with v₄ amount of Au octahedra with an edge length of 20 nm as by-products. To obtain the extinction spectrum of the sample, the original extinction spectra of the nanostructure and the by-products were simulated, and their weighted summation were used as the final spectrum of the sample. This final spectrum was normalized to the range of 0 to 1 for further data processing.

### 2.4.2 Simulated Chemical Space 2

In the second simulated chemical space, an extra dimension to control the doping of Ag into Au nanostructures was added as described in Section 2.3. The input variables were defined as (v₁, v₂, v₃, v₄, v₅) with (v₁, v₂, v₃) determining the geometry in the same way as that in the first simulated chemical space by Eq. (19), (20) and (21). v₄ modified the distribution of Ag in nanostructures by selecting the v_{DFC,2} for the dipole component function (Eq. (16), (17) and (18)). Since there are four values of v_{DFC,2} in creating the set of Au—Ag bimetallic nanoparticles, the h^{th} value of v_{DFC,2} was used for a given 14 through Eq. (22). The discrete values of v_{DFC,2} were sorted in descending order from 0.9 to 0.6 first.

$$h = 1 + ⌈ 3 {v}_{4} ⌋ \qquad ( 22 )$$

The effect of by-products was also introduced via v₅ through the same way as that in the first simulated chemical space. For a given (v₁, v₂, v₃, v₄, v₅), the sample is composed of (1−v₅) amount of the Au—Ag bimetallic nanostructures depending on (v₁, v₂, v₃, v₄) as described above, together with v₅ amount of Au—Ag bimetallic octahedra with an edge of 20 nm and v_{DCF,2}=0.9 as the by-products.

### 2.5 Exploration in the Simulated Chemical Space

The exploration algorithm was tested and benchmarked with Random Search on both simulated chemical spaces. Comparing to Random Search, the exploration algorithm based on MAP-Elites showed a better performance in both exploring the chemical space to find diversified samples and optimised the performance of individual elite.

### 2.5.1 Method

The behaviour space to classify samples was based on the position of the most prominent peak. First, the wavelength range of [0.4, 0.9] μm was discretized into 10 subregions, with a region width of 0.05 μm. Then for a given sampling point of (11, 12, 13, 14) (for simulated chemical space 1) or (v₁, v₂, v₃, v₄, v₅) (for simulated chemical space 2), the corresponding extinction spectrum was simulated to give the peak prominences and positions.

In searching the peaks, the lowest threshold for peak prominence was set to 0.01. If the total peak number was lower than 2, the sample was no further processed and discarded. The class index of these discarded samples was set as 0. Depending on which subregion the most prominent peak is located in, a class index was assigned to the sample (from 1 to 10 with increased wavelength). The fitness of the sample was further calculated through the percentage of the extinction area within w range near the most prominent peak, which enabled the search to get rid of other peaks except for the most prominent one (Eq. (23)).

$$F = \frac{{\int }_{{x}_{p e a k} - w}^{{x}_{p e a k} + w} {I}_{x} d x}{\int {I}_{x} d x} \qquad ( 23 )$$

where F is the fitness function, Iₓ is the absorption of the normalized UV-Vis spectrum at wavelength x, xₚₑₐₖ is the position of the most prominent peak, and w is a parameter to define the region near the peak. Here we set w to 0.05 μm, which has similar range to the absorption area of one peak.

An absorption boundary condition in the input space was defined so that if the input variable is smaller or larger than the lower or upper boundary, the variable will be replaced by the lower or upper boundary. This boundary condition was used in all the tests. The initial sampling number was 10. The batch size for one step was 23 for both exploration algorithm and Random Search, which is consistent with the batch size that will be used in the actual nanoparticle synthesis. In the exploration algorithm, among these 23 samples, 10 samples were mutated from the parent set, 10 samples were from crossover among parents with a further 40% chance of mutation and 3 samples were randomly generated in the input chemical space. This setting distributed resources equally to mutation and crossover, with a small portion of random sampling to avoid being trapped locally. In the mutation process, we sampled a vector from a multi-Gaussian distribution with a mean of 0 and the same standard deviation for all dimensions and then added this vector to the original sampling point. The standard deviations were 0.08 and 0.15 in the first and second simulated chemical space, respectively. The standard deviation is higher in the second chemical space due to its increased dimensionality and complexity. The estimated upper boundaries for both the mean fitness among all the elites and elite number were from a grid search with a 0.05 interval of mixture rate (v₄ for simulated chemical space 1 and v₅ for simulated chemical space 2) from 0 to 1 for all the shapes and composition in the nanoparticle set.

### 2.5.2 Class Distribution and Interconnectivity in the Simulated Spaces

In both spaces, for a given mixture rate, the input space was discretized because of the round functions in Eq. (19)-Eq. (22). Varying the input parameters within the discretized regions does not change the output spectrum. By assigning each region with a class index based on its spectrum (Section 2.5.1) and summing the volume of the regions belonging to the same class, the phase volumes of the classes in the input space can be calculated. It should also be noted that the volumes of the regions at the input boundaries of 0 and 1 are smaller considering the definition of the round functions (Eq. (19)-Eq. (22)).

The interconnectivity among classes can be estimated by the interconnectivity of the discrete regions. For example, to estimate the interconnectivity between class X and Y, we can obtain all the discrete regions that belong to class X and their neighbouring regions. The contact area of the neighbouring regions that belongs to class Y was a good estimation of the interconnectivity between class X and Y.

However, the chemical space is not discrete in the dimension of the mixture rate, and no discrete regions can be defined in this dimension. To estimate the volume in the input space and the interconnectivity of different classes, we sampled discretely in the dimension of the mixture rate with an interval of 0.05 from 0 to 1. Consequently, the discrete regions for both simulated spaces were created respectively. By summating the volumes of the regions belong to the same class, we estimated the phase volumes of different classes in the input space. By calculating the contact area from the neighbouring regions as described above, we estimated the interconnectivity among classes (see FIG. 16 and FIG. 17 for simulated chemical space 1 and 2 respectively).

### 2.5.3 Results

We randomly sampled 10 points as the initial data set. Then we further explore the simulated chemical space 1 and 2 for another 50 and 200 steps respectively. Since both the exploration strategy based on MAP-Elites and Random Search contain stochastic effects from choosing random variables, the search was repeated 16 times to elaborate their capability in exploring the chemical space. In each repeat, the mean fitness among all the elites and the elite number were measured.

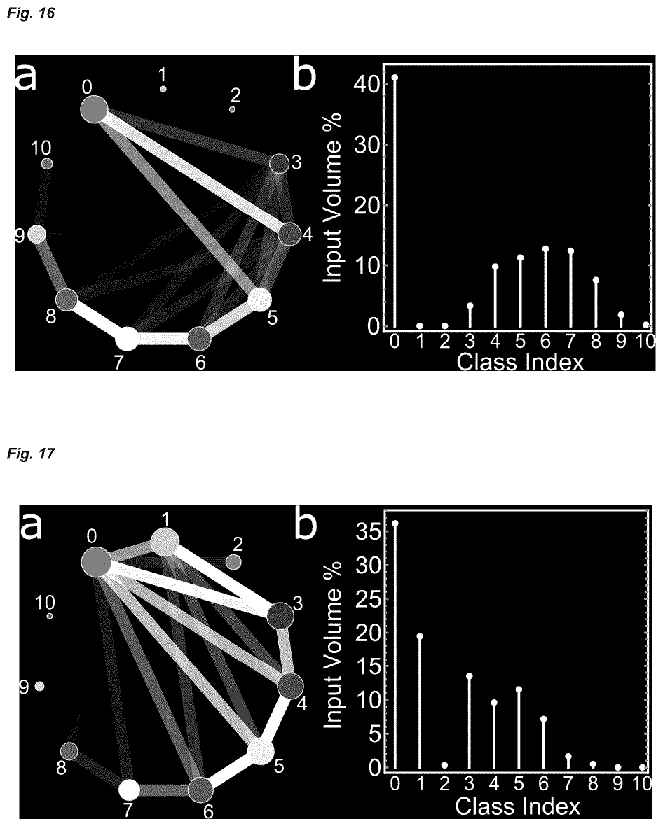

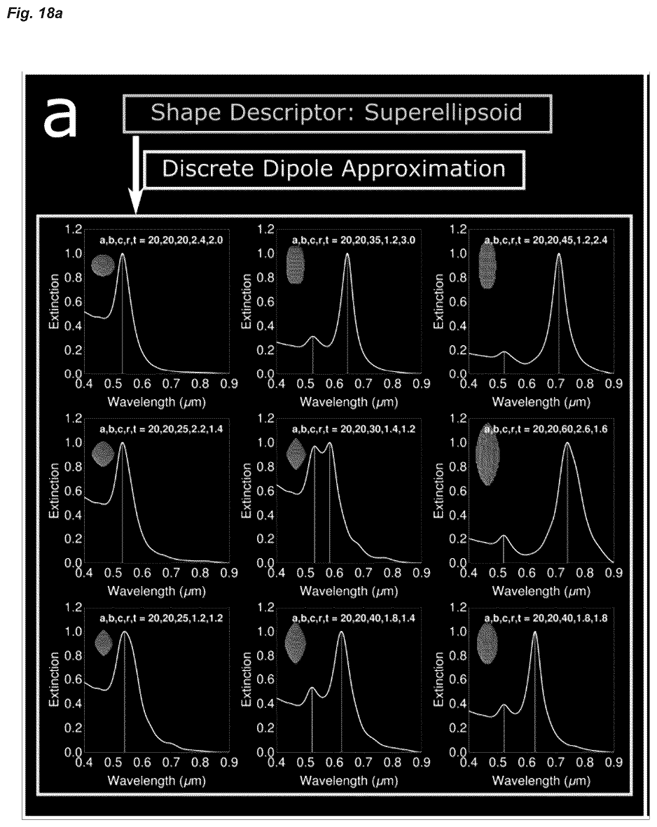

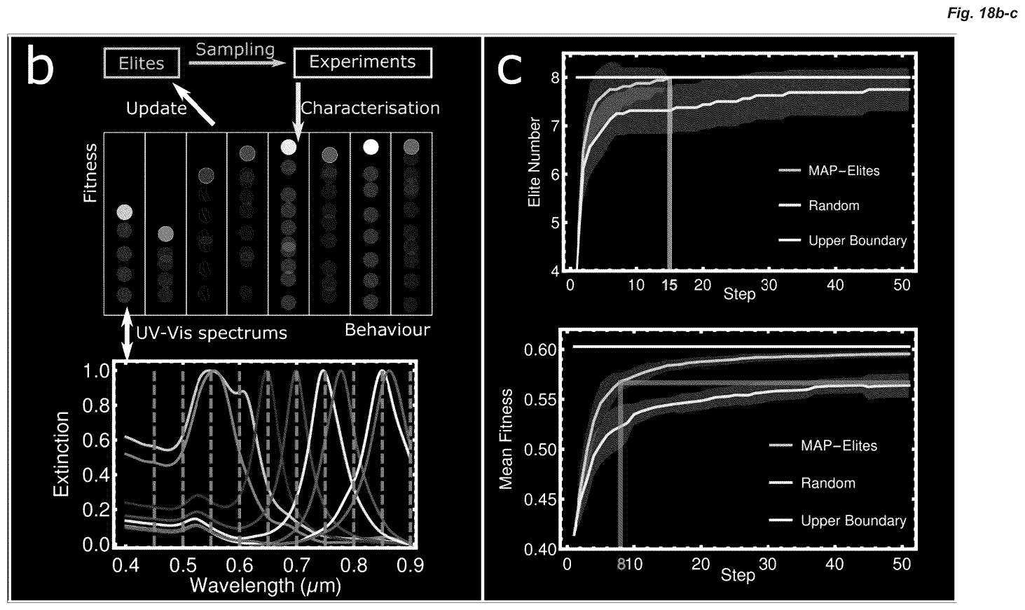

The results from simulated chemical space 1 are shown in FIG. 18 together with their standard deviation for every step. The upper boundaries are estimated from the grid search. The mean fitness is defined by averaging the fitness values of different elites. In looking for elites belonging to different classes, Random Search almost converged after 51 steps and still cannot find all kinds of elites in the space, while with the exploration algorithm, all the possible elites were found after 15 steps. And the average mean fitness of Random Search at the end (0.564±0.012) is lower than both that from the exploration algorithm (0.595±0.003) and the estimated upper boundary (0.603). The exploration algorithm exceeded the final average mean fitness of Random Search within 8 steps and ended up with an average mean fitness ca. 99% of the estimated upper boundary.

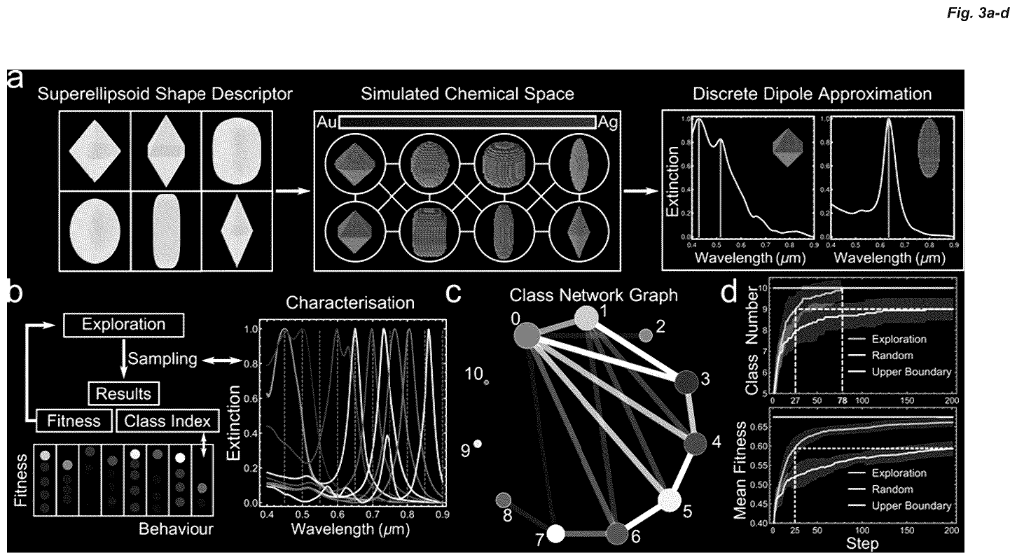

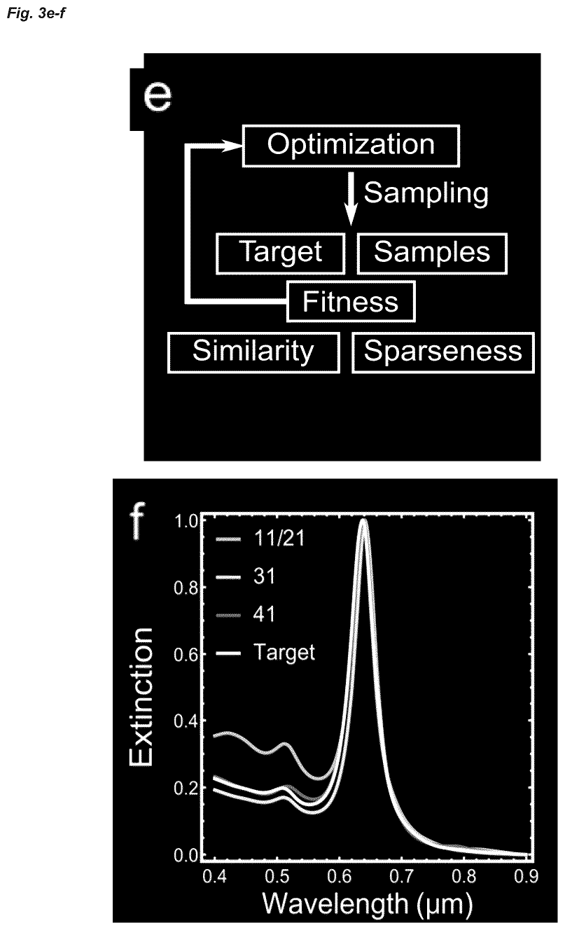

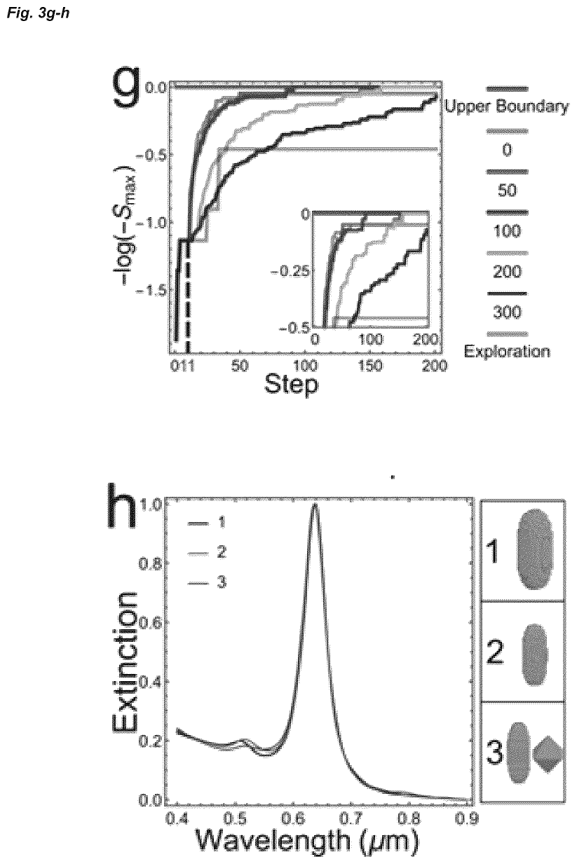

The results from exploring chemical space 2 are shown in FIG. 3, where we found a set of high-quality and diversified UV-Vis spectra. Since this space is more complicated, after the initial sampling, the number of steps for exploration was increased from 50 to 200. After the exploration, Random Search still cannot find all the elites, with a large standard deviation of 0.5 among the repeats. The exploration algorithm outperformed it after 27 steps and can find all the elites after 78 steps (FIG. 3e top). After 25 steps, the average mean fitness from the exploration algorithm exceeded the final mean fitness from Random Search (0.593±0.019), and eventually reached 0.661±0.010, which is ≈0.014 smaller than the upper boundary (0.675) (FIG. 3e bottom) and ca. 98% of it.

This test benchmarked the capability of the exploration algorithm in searching the feature space over Random Search, showing it can return a set of high-performance and diversified solutions. When we applied the exploration algorithm in real experiments in an autonomous platform, more modifications were introduced and will be discussed in the following sections.

### 2.6 Optimisation in the Simulated Chemical Space

After the exploration, the fine-tuning the optical properties of the nanoparticles to approach a given target was necessary. Here we implemented the optimisation algorithm based on global search with local sparseness (GS-LS) and utilised the evolutionary algorithm (EA) as the optimiser after the space was explored. With the optimisation algorithm, it is demonstrated that not only the global maximum, but also multiple optimal solutions of different nanostructures can be found successfully by considering the local sparseness in the simulated space.

### 2.6.1 Method

The goal of optimisation is to find the sample with the highest fitness but also several other samples with moderate fitness that are separated in the input space. In our experiment, the fitness (F) (Eq. (3)) was a linear summation of the local sparseness term (Eq. (1)) and the similarity metric (M_{S}) (Eq. (2)). The local sparseness term was used to quantify the local sampling density (Eq. (1)) while the similarity metric measures the absolute spectrum difference as well as the peak position difference of the highest peak (Eq. (2)).

$$S ( x ) = \frac{1}{K} {\sum }_{i = 1}^{K} d i s t ( x , {y}_{i} ) \qquad ( 1 )$$

where dist(x, y) measures the distance between x and y in the input space, and yᵢ is the i^{th} closest sample to x.

$${M}_{S} = - \vert p - {p}_{t a r g e t} \vert - {k}_{1} {\sum }_{i} \vert {I}_{x , i} - {I}_{t a r g e t , i} \vert + {k}_{2} \qquad ( 2 )$$

where p and p_{target} are the peak positions of the highest peak in the UV-Vis spectra of the sample and the target. I_{x,i} and I_{target,i} are the i^{th} intensity of the UV-Vis data of the sample and the target respectively. k₁ is used to tune the importance between constraining the peak position and increasing the overall similarity between the spectra, and was set as 0.2. It should be noted the unit of the wavelength (|p−p_{target}|) here is micrometer, thus this term is trivial and optimisation was mainly aimed to find the exact same target spectrum, which was different from the experimental optimisation later, where we focused more to find samples with the same peak positions. When two identical UV-Vis spectra are found, both |p−p_{target}| and Σᵢ|I_{x,i}−I_{target,i}| reduce to 0 and k₂ puts an upper boundary of the similarity metric, which was set as 1 when plotting the fitness function.

$$F = {M}_{S} + {k}_{3} S \qquad ( 3 )$$

where S is the local sparseness term defined in Eq. (1). k₃ is used to tune the importance of the local sparseness and are varied from 0 to 300 with an interval of 50 considering the scale of S in the benchmark (See below).

The optimisation was conducted in simulated chemical space 2. The same absorption boundary condition in the input space as that in the exploration algorithm was used. Exploration was conducted as described above to see if it was able find the intended target. For benchmarking, the optimisation started after 11, 21, 31 and 41 steps of the exploration (including the initial random sampling in the exploration algorithm), and stopped when a complete 201 steps including both exploration and optimisation was reached. The data from exploration were used as the initial dataset for optimisation.

In optimisation, the five samples with the highest fitness (Eq. (3)) from all the available data (including those from previous steps) were selected as the parents for crossover and mutation. Ten unique nearest neighbours (including the sample itself) are used to calculate the local sparseness. The local sparseness term was updated from step to step. There are 23 samples generated per step and among these 23 samples, 10 samples were mutated from the parent set, 10 samples were from crossover among parents with a further 40% chance of mutation and 3 samples were randomly generated in the input chemical space. In the mutation process, a vector from a multi-Gaussian distribution with a mean of 0 and a standard deviation of 0.15 for all dimensions was sampled and then added the original sampling point. Note all the UV-Vis spectra were normalized before data processing. The upper boundary of the similarity metric is k₂, which is set as 1.

One target spectrum was set for the benchmark. Its parameters to control the nanostructure was listed in Table 2-1. The way that chemical space 2 was defined makes the similarity landscape intrinsically flat (e.g., varying v₁ from 0.6875 to 0.8125 does not change the similarity, because it is that matters). The target spectrum can be achieved by sampling in a region and the variable ranges of this region are also listed in Table 2-1. Note that v₅ defines the mixture rate and is continuous.

TABLE 2-1
The corresponding parameters of (c, r, t)
to define the nanostructure for the
target spectrum. The input parameter
ranges that correspond to the target
in the optimisation.

| Target | c (nm) | r | t | ν_{DCF,2} | ν₅ |
|---|---|---|---|---|---|
| 1 | 50 | 2.6 | 2.6 | 0.9 | 0 |
| Target | ν₁ | ν₂ | ν₃ | ν₄ | ν₅ |
| 1 | [0.6875, | [0.75, | [0.75, | [0, | 0 |
| | 0.8125] | 0.85] | 0.85] | 0.16667] | |

The input difference between the highest-performance sample and the input variable range of target (Table 2-1) was monitored during the optimisation. From v₁ to v₄, if the value of the sample is within the variable range of the target, the difference is 0. If the input value of the best sample is out of the range, its absolute differences with both the upper and lower boundaries were calculated, and the smaller value was taken as the difference. For v₅, the absolute difference is calculated normally. The difference will be discussed below.

### 2.6 Results

Although exploration based on MAP-Elites can find the elites with optimal absorption peaks, it is not enough to fine-tune the optical properties due to the wavelength window when we defined the subregions (e.g., if we require the target with an absorption peak at 654 nm, the algorithm will treat all the samples with absorption peak from 650 nm to 700 nm as the same class). Furthermore, it distributes the resources to the exploration and parallel optimisation of the multiple elites. Therefore, purely exploration can increase the UV-Vis similarity to the target but will converge after several steps and is unlikely to find the global maximum.

An optimiser that aims to increase the similarity between the sample and the target is necessary if we want to fine-tune the optical properties. However, UV-Vis spectrum is not a unique characteristic of the nanostructures and multiple different structures can share similar UV-Vis signals. Thus, many local maxima regarding the similarity can coexist in the chemical space, each of which is as valid as the others. By increasing the weight of the local sparseness, the search was encouraged in less-sampled regions further, which helps to avoid being trapped in any single local maximum. However, if the weight of the local sparseness is too high, the algorithm prefers the region with less samples and focuses less in increasing the similarity, while increasing the similarity metric is the actual task. The phenomena were observed during the benchmark.

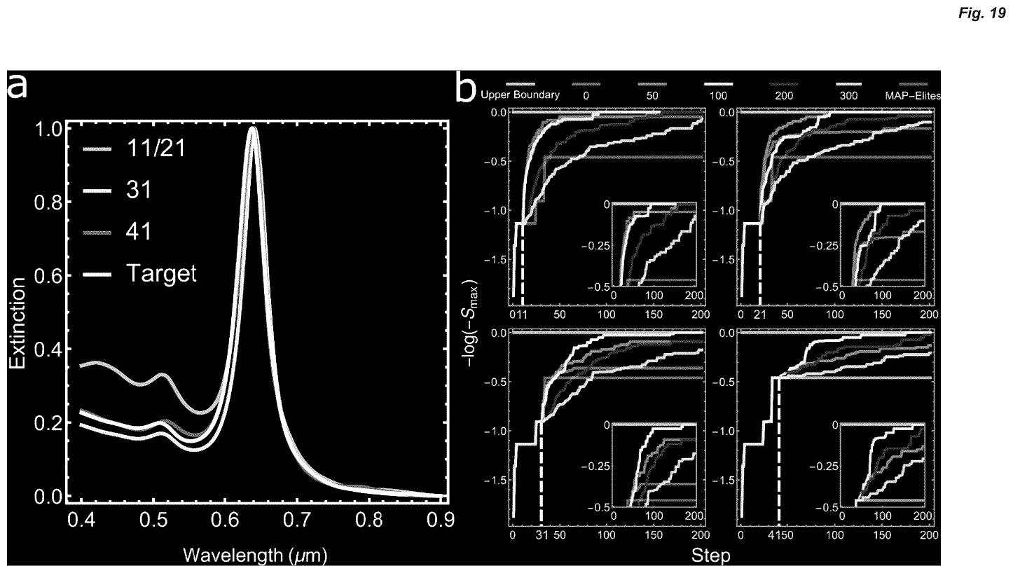

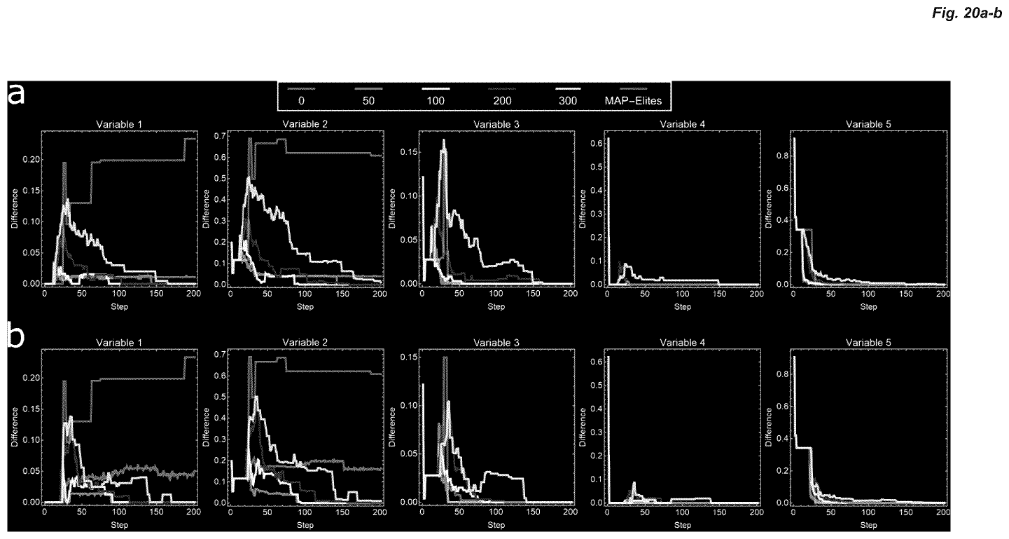

FIG. 19 shows the results of optimisation towards the target. Initial data set for optimisation was created by running exploration for 11, 21, 31, 41 steps respectively. The total step number including both exploration and optimisation was controlled to be 201. The exploration algorithm was also run for 201 steps as a reference, showing it cannot sufficiently reach the global maximum regarding the similarity metric. All the tests of the optimisation algorithm were repeated 16 times independently with the same initial data set, and the average results of the repeats are shown. FIG. 19a shows the target and the most similar UV-Vis spectra to it after 11, 21, 31 and 41 steps of exploration. FIG. 19b shows the increase of the highest similarity metric from the samples found by the optimisation algorithm with varied sparseness coefficient k₃ and initial data set during the optimisation. When the k₃ was set as 0, the local sparseness did not functionalize at all, and the algorithm became GA. The solutions were trapped in the local maximum due to the complexity of the search space and the bias from the initial data set. With more data points from exploration, the final similarity after optimisation was decreased because more local maxima acted as the parents and tended to damp the search. When k₃ was set as 50, the local sparseness term encouraged the global search. Thus, the efficiency of finding global maximum can be increased (see the case with starting step of 21). But it is still possible to be trapped in the local maxima during the optimisation, which damps the overall efficiency (see the case with starting step of 11) or forbids the convergence to the global maximum (see the cases with starting step of 31 or 41). When k₃ was set as 100, the optimisation algorithm can always find the global maximum efficiently. When k₃ was further increased to 200 or 300, it tended to explore the unsampled region more, so the increase of the similarity metric is slower. The average absolute differences (among 16 repeats) for the five variables in the input space during the optimisation are shown in FIG. 20.

Another important purpose of the optimisation algorithm is to find multiple solutions with similar UV-Vis but are separated in the input space (v₁, v₂, v₃, v₄, v₅), which represents the local maxima in the similarity landscape. Here we will analyse the result from one repeat where the starting step of optimisation was 11 and k₃=100.

Because the implementation of the round function (┌vᵢ┘, where vᵢ is an input space variable for i from 1 to 4), the chemical space is intrinsically flat, i.e., samples within a vicinity in the input space can have the same nanostructure and mixture rate, so that the same similarity metric. This feature created multiple local maximum regions instead of points in the input space. To check the structural diversity of the samples from the optimisation, sampling points corresponding to the same nanostructure and mixture rate from the same vicinity were removed. K-nearest neighbour criteria was used to filter solutions that are close to the same local maximum. After that, sampling points were selected as the solutions so that the solution's similarity metric is the highest among its ten nearest neighbours in the input space.

A point considered here is whether these local maximum regions sampled by the optimisation strategy? Also considered is whether the K-nearest neighbour filtering strategy increase the structural diversity in the solutions.

Since the first four variables in the input space (v₁, v₂, v₃, v₄) controls the nanostructure according to Eq. (19) to (22), the nanostructure parameter space can be defined as (c, r, t, v_{DCF,2}, v₅), where v₅ still represents the mixture rate. The local maxima in this parameter space correspond to the local maximum regions in the input space. To find the local maxima, grid search was conducted in all the possible combinations of (c, r, t, v_{DCF,2}) as well as v₅ in the range from 0 to 1 with an interval of 0.05. The following analysis will be in the parameter space of (c, r, t, v_{DCF,2}, v₅).

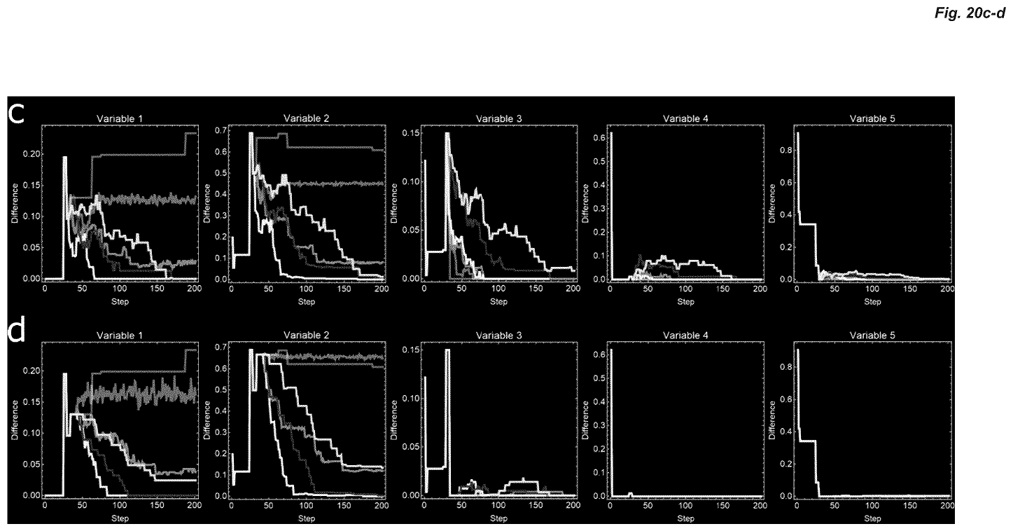

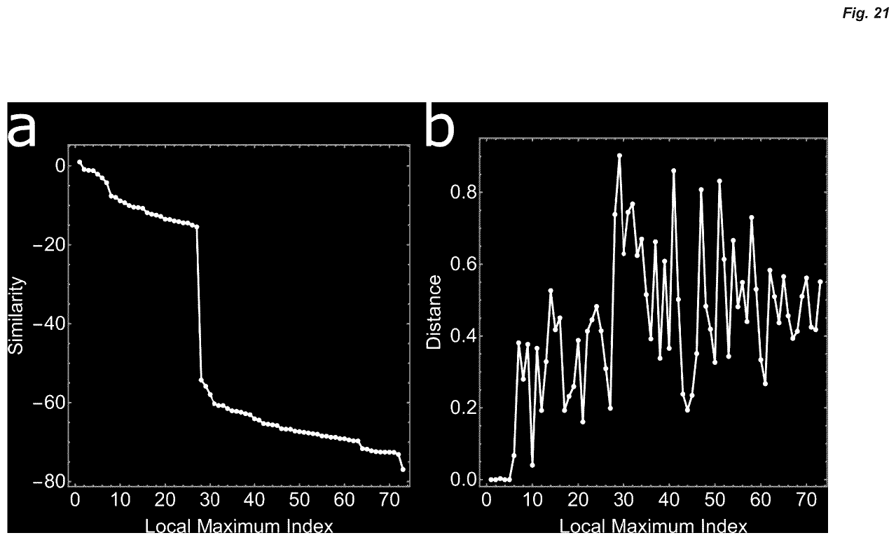

Multiple local maxima (73 in total) exist in this simulated space. The optimisation was guided by the similarity metric so that local maxima with higher similarity are more likely to be found. The distance between the local maximum and its closest solution (structure) indicates how well this local maximum is optimised. The local maxima were sorted in a descending order regarding their similarity metrics (FIG. 21a), and the increased distances of these local maxima to the closest samples are shown in FIG. 21b. These results indicated the preference of searching local maxima with higher similarities during the optimisation. In calculating the distance, to make the scale consistent, every dimension in the space of (c, r, t, v_{DCF,2}, v₅) was normalized from 0 to 1 by a linear transformation.

The top five local maxima with the highest similarity metrics are shown in Table 2-2. To check if the optimisation algorithm found these local maxima, the solutions that are closest to them are shown in Table 2-3, with neglectable difference from the local maxima (only a difference of 0.030 in of v₅ in solution 4).

TABLE 2-2
The local maxima in the nanostructure parameter space and
their corresponding local maximum regions in the input space.

| Local | | | | | |
|---|---|---|---|---|---|
| maximum | c (nm) | r | t | ν_{DCF,2} | ν₅ |
| 1 | 50 | 2.6 | 2.6 | 0.9 | 0 |
| 2 | 40 | 1.2 | 2.6 | 0.9 | 0 |
| 3 | 40 | 1.0 | 2.2 | 0.8 | 0.1 |
| 4 | 45 | 1.6 | 2.6 | 0.9 | 0 |
| 5 | 45 | 1.4 | 2.2 | 0.9 | 0 |
| Local | | | | | |
| maximum | ν₁ | ν₂ | ν₃ | ν₄ | ν₅ |
| 1 | [0.6875, | [0.75, 0.85] | [0.75, 0.85] | [0, | 0.0 |
| | 0.8125] | | | 0.16667] | |
| 2 | [0.4375, | [0.05, 0.15] | [0.75, 0.85] | [0, | 0.0 |
| | 0.5625] | | | 0.16667] | |
| 3 | [0.4375, | [0.00, 0.05] | [0.55, 0.65] | [0.16667, | 0.1 |
| | 0.5625] | | | 0.50] | |
| 4 | [0.5625, | [0.25, 0.35] | [0.75, 0.85] | [0, | 0.0 |
| | 0.6875] | | | 0.16667] | |
| 5 | [0.5625, | [0.15, 0.25] | [0.55, 0.65] | [0, | 0.0 |
| | 0.6875] | | | 0.16667] | |

TABLE 2-3
The parameters of the nanostructures from
the solutions closest the five local
maxima. Other parameters to control the
nanostructure are constant: α = b = 20 nm
and V_{DCF,1} = 50.

| Solution | c (nm) | r | t | ν_{DCF,2} | ν₅ |
|---|---|---|---|---|---|
| 1 | 50 | 2.6 | 2.6 | 0.9 | 0.0000 |
| 2 | 40 | 1.2 | 2.6 | 0.9 | 0.0000 |
| 3 | 40 | 1.0 | 2.2 | 0.8 | 0.1030 |
| 4 | 45 | 1.6 | 2.6 | 0.9 | 0.0000 |
| 5 | 45 | 1.4 | 2.2 | 0.9 | 0.0000 |

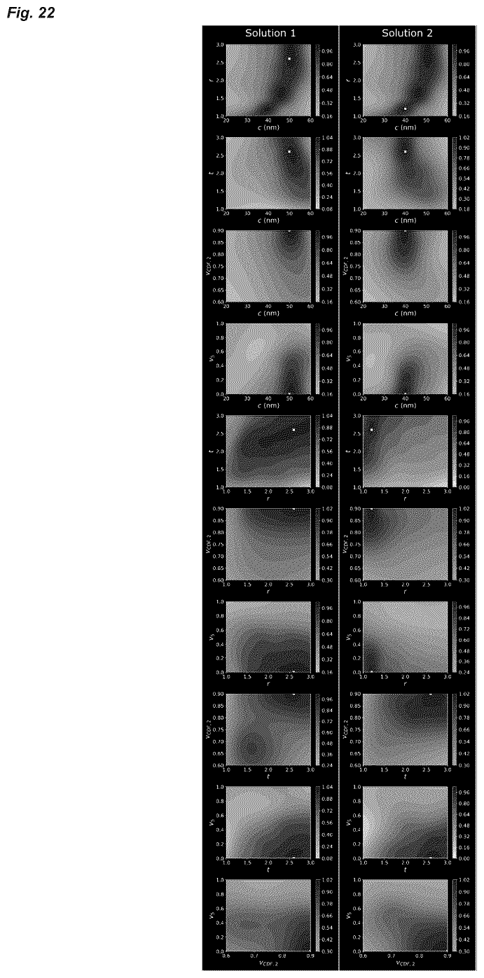

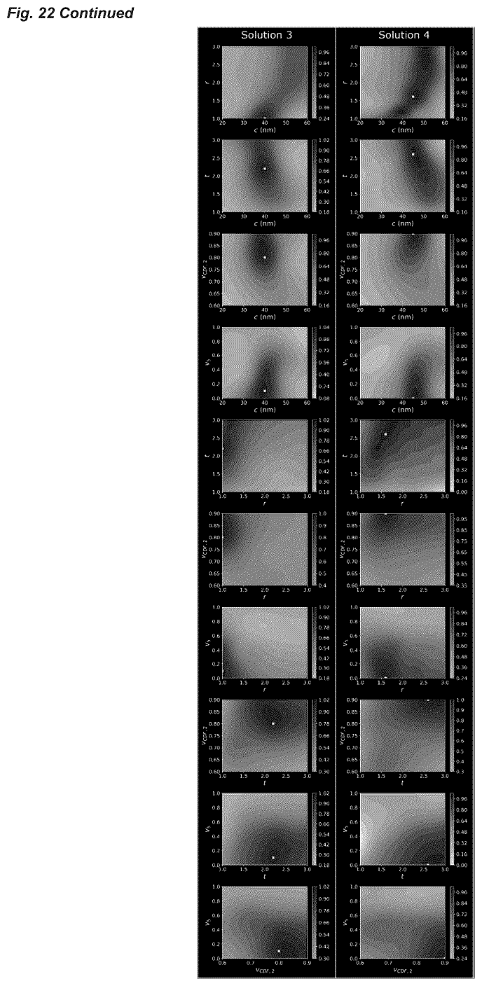

To visualize top five local maxima and their corresponding solutions in the high dimensional space of (c, r, t, v_{DCF,2}, v₅), a Gaussian process (GP) was trained with the data from optimisation. For every local maximum, the similarity distribution on the planes that composed of any of the two dimensions and pass the local maximum are shown in FIG. 22. The local maxima are indicated by the red points. Note the range of the similarity metrics was normalized from 0 to 1 by a linear transformation. The nearest solution to the local maximum was projected into the plane and indicated by the cross mark. These results demonstrate the successful search of high-performing local maxima with the optimisation strategy.

TABLE 2-4
The input variables and their corresponding parameters of the
nanostructures from the solutions when the starting step of was 11
and k₃ = 100. Other parameters to control
the nanostructure are constant: α = b = 20 nm and v_{DCF,1} = 50.

| Solution | ν₁ | ν₂ | ν₃ | ν₄ | ν₅ |
|---|---|---|---|---|---|
| 1 | 0.8044 | 0.8441 | 0.8133 | 0.0000 | 0.0000 |
| 2 | 0.6891 | 0.8441 | 0.8133 | 0.1035 | 0.0409 |
| 3 | 0.7732 | 0.7472 | 0.7332 | 0.1155 | 0.0000 |
| 4 | 0.4764 | 0.0989 | 0.8084 | 0.0000 | 0.0000 |
| 5 | 0.4583 | 0.0000 | 0.5670 | 0.4820 | 0.1030 |
| Solution | c (nm) | r | t | ν_{DCF,2} | ν₅ |
| 1 | 50 | 2.6 | 2.6 | 0.9 | 0.0000 |
| (Local maximum 1) | | | | | |
| 2 | 50 | 2.6 | 2.6 | 0.9 | 0.0409 |
| 3 | 50 | 2.4 | 2.4 | 0.9 | 0.0000 |
| 4 | 40 | 1.2 | 2.6 | 0.9 | 0.0000 |
| (Local maximum 2) | | | | | |
| 5 | 40 | 1.0 | 2.2 | 0.8 | 0.1030 |
| (Local maximum 3) | | | | | |

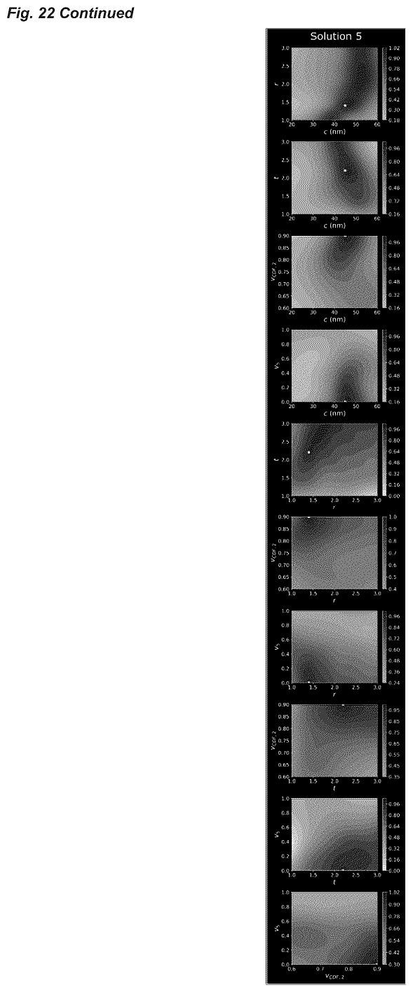

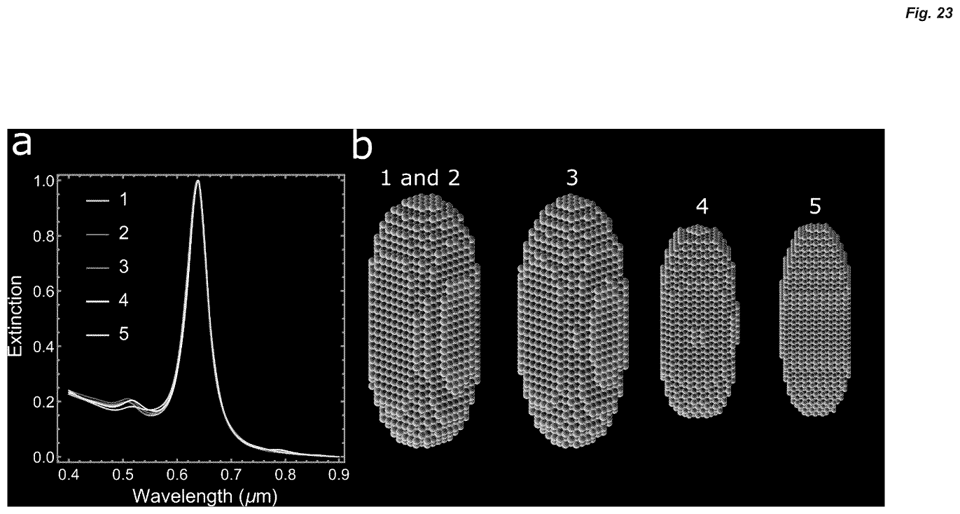

Despite the successful search of the multiple local maxima, multiple sampling points can correspond to the same local maxima. Simply sorting the sampling points according to their similarity does not enable the diversity in the solution set. Another step of filtering out the sampling points near the same local maximum to get a set of solutions with more chance to correspond to more different local maxima is necessary. Here we used the K-nearest neighbour criteria. The solutions were selected so that every solution has the highest similarity among its ten nearest neighbours (including itself). Without the ten-nearest neighbour criteria, the top five solutions with the highest fitness are all in the vicinity of the global maximum. With the ten-nearest neighbour criteria, the five samples correspond to three different local maxima (including the global maximum). The input variables of the solutions and their corresponding nanostructure parameters are shown in Table 2-4. Their UV-Vis spectra and the discrete dipole representation of the nanostructures without by-products are shown in FIG. 23.

To further measure the diversity of the solution set, both the number of the unique local maxima (N_{L}(x)) and the least sampling number (Nᵢ(x)) were defined as following:
For every solution, the local maximum that is closest to it is recorded.
1. N_{L}(x): the number of the unique closest local maxima to the first x solutions.
2. Nₗ(x): at least the first Nₗ solutions need to be selected so that the number of the solutions' closest local maxima is no less than x.

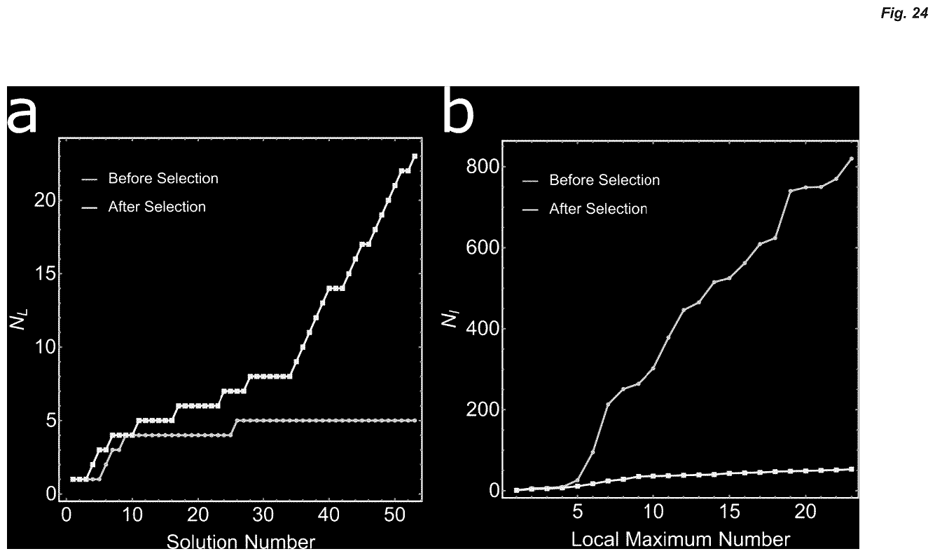

The two functions of N_{L}(x) and Nₗ(x) were compared before and after the selection of ten-nearest neighbours (FIG. 24). With the selection criteria, the number of unique local maxima (N_{L}) closest to the given top x solutions was increased (FIG. 24a). The required number of solutions (Nₗ) so that they correspond to different local maxima was decreased (FIG. 24b). Both results demonstrated the increased solution diversity from the vicinity of different local maxima after the implementation of K-nearest neighbour criteria.

### 3. Exploration of the Experimental Chemical Spaces in the Seed-Mediated Synthesis of Au Nanoparticles

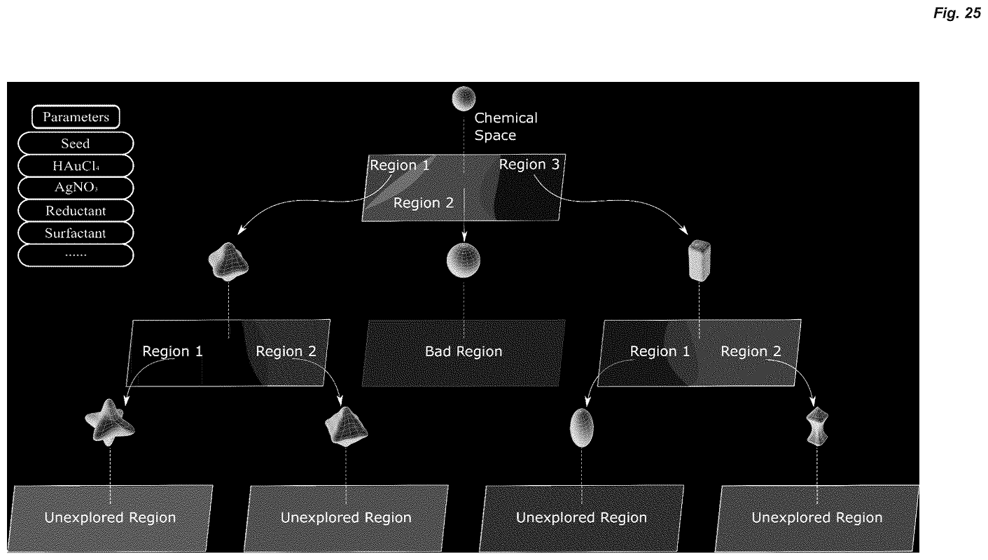

A multistep growth strategy was applied to engineer and explore Au nanostructures (FIG. 25). With the seed-mediated method and overgrowth of Au nanoparticles, multiple hierarchically-linked chemical spaces are generated and investigated. The resulting nanoparticles discovered in a given chemical space can be selected and used as seeds for the next level exploration in the hierarchical series. The exploration algorithm based on MAP-Elites as described above was implemented to investigate three chemical spaces to get a diversified set of nanostructures.

Instead of detecting the nanostructures directly with TEMs, UV-Vis spectra were used to indicate the diversity of nanostructures during the search. The flexible manipulation of various UV-Vis features was demonstrated by varying the definition of classes and fitness in the exploration of the three chemical spaces, which resulted in the emergence of multiple uniquely-shaped nanoparticles (Section 3.2, Section 3.3 and Section 3.4).

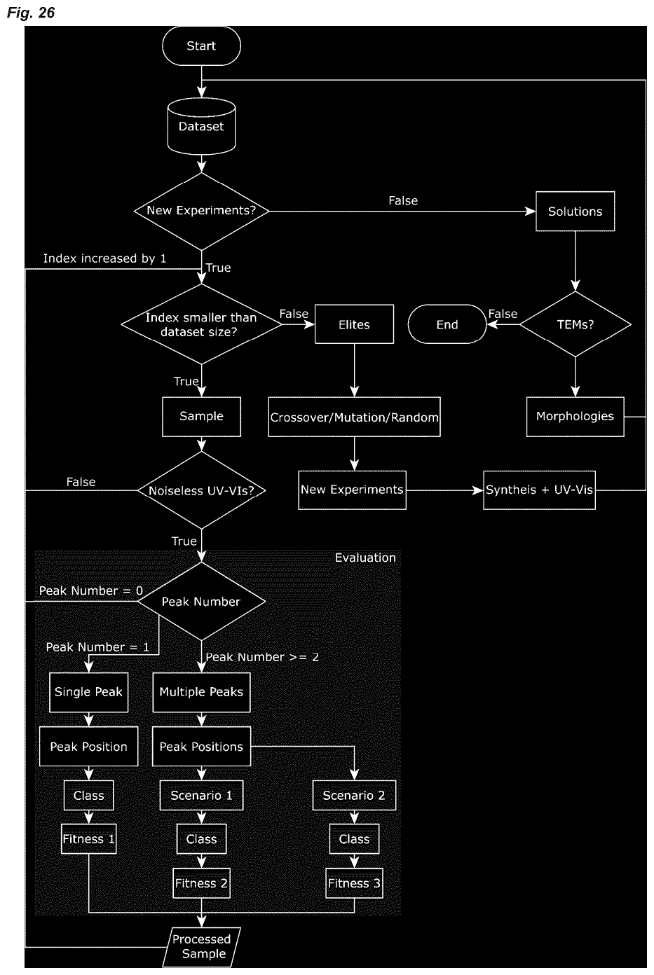

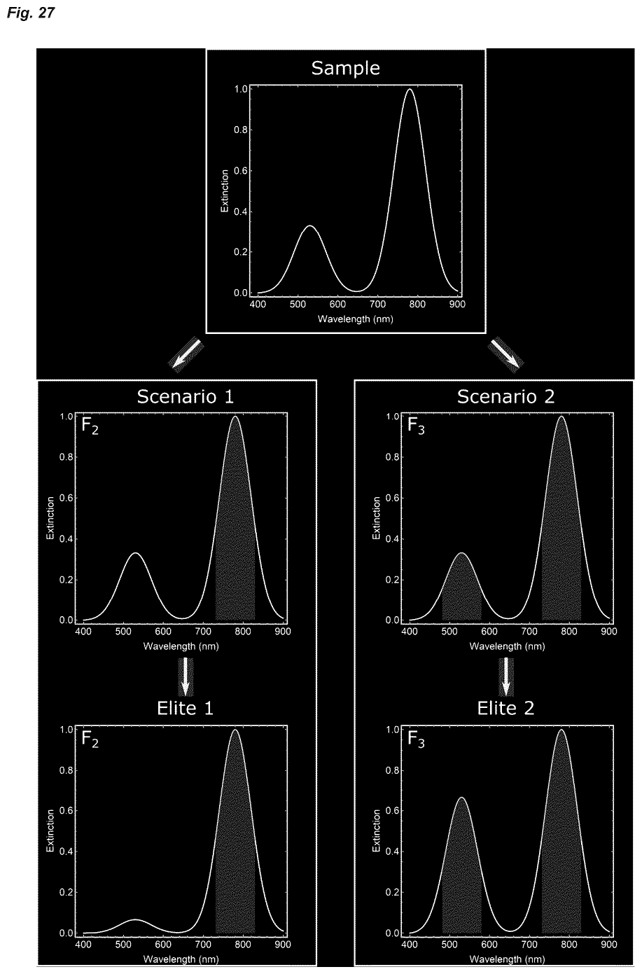

The flow diagram of the exploration strategy is shown in FIG. 26. The dataset is updated during the investigation and used to design new experiments. If there is not prior knowledge of the chemical space and the dataset is empty, random sampling will be applied in designing the first batch of experiments. Otherwise, the samples in the dataset will be evaluated to allocate their classes and fitness with certain criteria, which will generate new experiments. The evaluation of the samples is described below:
1. The sample's UV-Vis spectrum is obtained and will be further processed if the following two criteria are met:
   a. The sample spectrum is not too noisy.
   b. The UV-Vis peak number is >0.
2. If there is only one peak, the sample has the single peak feature and belongs to the single peak system. The full spectral wavelength range is discretized into multiple subregions first and depending on which subregion the sample's peak is located in, a class index will be assigned to it. Its fitness will be calculated further (F₁).
3. If there are more than two peaks, the sample has the multiple peak features and belongs to the multiple peak system. The most prominent two peaks will be considered. Again, the full spectral wavelength range is discretized into multiple subregions. The subregions which the sample's most prominent and second most prominent peaks are in will be recorded. Two different fitness functions (F₂, F₃) are calculated independently for a given sample. These fitness functions correspond to two different scenarios: scenario 1 to focus on the most prominent peak, making it the only signal in the spectrum and scenario 2 to encourage the continued production of multiple signal peaks. Two class indexes were assigned to the sample depending on the allocated subregions for each peak, and the chosen fitness function. For example, considering a sample (x) with its two peaks in the {i^{th}, j^{th}} subregion, two fitness values were calculated (F₂, F₃) respectively. Then two class indexes as {i^{th}, j^{th}, 1} and {i^{th}, j^{th}, 2}, which indicate the different scenario, can be assigned to the sample (See FIG. 27).

The samples in the dataset were evaluated as described above. For every unique class, the sample with the highest fitness in the class was selected and designated as an elite. The elites from different classes defined the elite set. This elite set was used as the parent set in designing new experiments. The new experiments generated by a combination of crossover and mutation processes within the elite set, and also a small portion of random sampling. The single-peak and multiple-peak systems can be explored simultaneously, sequentially or selectively by constraining the classes that can be added to the elite set. The new experiments are conducted by the autonomous platform as described in Section 1. The process including conducting experiments, analysing data and generate new experiments iterated many times as needed.

The evaluation criteria of classification and fitness are essential because they directly affect the selection of the elites (parents), thus the exploration. Various general criteria can be defined dependent on the UV-Vis features we wish to explore, all of which will be demonstrated with the three chemical spaces detailed below. These criteria can be either static or dynamically changed during the exploration process. For example, considering the exploration of the single-peak system, the exploration can be focused by increasing the number of subregions in an area of interest within the spectral range whilst decreasing this number elsewhere. This change increases the number of the elites with a particular desired behaviour, thus allocates more resources to increasing the performance of these elites. It makes the exploration more focused on elites with specific behaviours by scarifying the overall diversity of the elites. It can be done with greater confidence as the exploration proceeds and areas of the space are revealed to be less interesting.

Transmission electron microscopy offered detailed information of nanostructures directly and can be used to add extra information to the dataset after exploring the space for enough steps. The information from TEM images can help to modify the classification criterion to constrain the exploration for specific UV-Vis features. It can also help to modify the fitness function in a more explicit way, searching for samples which were difficult to reach before. This dynamic process was illustrated in exploring the first chemical space in three stages, each of which will be discussed in Section 3.2.

### 3.1 Chemical Reagents

All stock solutions were made using Type I ultrapure water (18.4 MΩ·cm).
Hexadecyltrimethylammonium bromide, >99%, Acros Organics, CTAB.
Hexadecyltrimethylammonium chloride, >99%, Acros Organics, CTAC.
Gold (III) chloride trihydrate, 99.9%, Sigma-Aldrich, HAuCl₄.
Ascorbic acid, 99.9%, Sigma-Aldrich.
Silver nitrate, 99.9999%, Sigma-Aldrich, AgNO₃.
Hydroquinone, 99.5%, Acros Organics.
Hydrochloric acid, ACS Reagent, 37%, Honeywell Fluka™, diluted with Type I water.
Sodium hydroxide, 98%-100.5%, Honeywell Fluka™, NaOH.
Orion™ pH buffer solutions (4.0, 7.0 and 10.0), Thermo Scientific™

### 3.2 Chemical Space 1: Seed-Mediated Synthesis on Cuboctahedron Single Crystals

The first chemical space was based on the 2 nm Au seed. It served as the starting point of the exploration in the multistep growth. The reagent concentrations were tuned to suit the volumes of the syringe pumps.

### 3.2.1 Experimental Details

### Synthesis of the 2 nm Au Seed:

1. HAuCl₄ (5 mL, 0.5 mM) and CTAB (5 mL, 0.2 M) were mixed in a 14 mL vial.
2. Under vigorous stirring ice-cold NaBH₄ (0.6 mL, 0.01 M) was added to the solution. The solution was stirred for 5 minutes.
3. The solution was aged for 25 minutes at 30° C.
4. The seed solution was diluted by adding 49.4 mL Type I ultrapure water for the accurate pump volume transfer.
   Synthetic procedure. Each reaction was performed with the followed order of addition by the platform, using volumes provided by the algorithm:
1. CTAB (0.2 M) added.
2. HAuCl₄ (1.0 mM) added.
3. AgNO₃ (0.25 mM) added.
4. Ascorbic acid (3.3 mM) added.
5. 10 seconds allowed for the reduction to complete.
6. Total volume constrained by addition of water if necessary.
7. 2 nm Au seed (0.50 mL) added.

The overall volume of the synthesised sample was constrained to 12.00 mL with boundary conditions in the algorithm and addition of water. The boundary conditions will be discussed below. The volume of the seed solution for each reaction performed was fixed at 0.50 mL.

Each reaction solution was stirred during the synthetic procedure listed above. Once the synthetic procedure was over, all the solutions were kept undisturbed for 1 hour to complete the growth process.

### UV-Vis Characterisation and Data Processing:

All the samples from experiments were characterised with the QE-PRO UV-Vis spectrometer from Ocean Insight Ltd. The raw data was normalized to the range from 0 to 1, directed through a low-pass filter (with a passing frequency of 15) to remove noise, normalized again and further interpolated with a cubic spline. The average absolute difference before and after passing through the low-pass filter was calculated and if it was larger than a threshold (0.005), we regarded this sample as too noisy and discarded it. Samples without detectable peaks or with the largest peak prominence less than 0.2 were also discarded. The threshold of detecting peaks was set as 0.02 in the experiments.

### TEM

Carbon coated 400 mesh copper grids (Agar Scientific/product code AGS160-4) were glow discharged using a Quorum Q150T ES high vacuum coater. Samples (5 μL) were drop cast onto the carbon film surface and left to dry. JEOL1200 EX TEM was used at 80 kV. Images were captured using a Cantega 2k×2k camera and Olympus ITEM software.

### Stability of the System

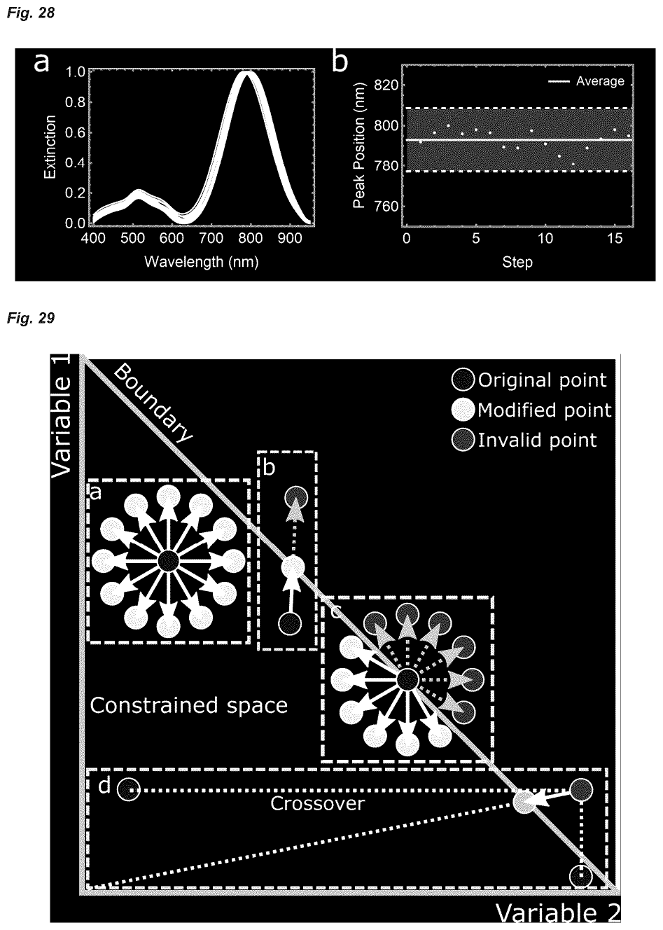

To monitor and demonstrate the platform's ability to consistently reproduce results, 1 of 24 vials on the CRM was used to perform an identical reaction (Table 3-1) during each step of the entire 16-step exploration. These 16 samples were analysed alongside the exploration samples and served as standards for the stability of the whole system and the stock solutions being used. The spectra corresponding to these samples are shown in FIG. 28a, with small differences in the shape yield and shift in the peak of the longitudinal mode (FIG. 28b). Given the chemical sensitivity in nanomaterial synthesis, such minor differences can be attributed to inherent variation in chemistry as these are growth processes rather than strict mechanistic processes as well as the slight inconsistency of robotic operations, temperate, stock solutions and UV-Vis spectrometers, etc., but were found to be negligible on this system and consistently monitored.

TABLE 3-1

| | CTAB | HAuCl₄ | AgNO₃ | Ascorbic | Water | Seed |
|---|---|---|---|---|---|---|
| Standard | (mL) | (mL) | (mL) | acid (mL) | (mL) | (mL) |
| 1 | 4.40 | 2.50 | 1.80 | 1.10 | 1.80 | 0.50 |

The input parameters as the standard samples to test the stability of the autonomous platform in chemical space 1. The concentrations of the reagents are available in the same section above.

### 3.2.2 Data Processing and Algorithm

To explore the chemical space and obtain the nanostructures with high qualities, the exploration algorithm was customized from several aspects including adding extra operations in mutation and crossover to satisfy the new boundary conditions, changing the definition of different classes in the behaviour space and modifying the fitness function accordingly.

### Input Space, Boundary Conditions and Sampling Strategy

The input space was defined by the normalized variables with a range from 0 to 1 derived from the chemical reagent volumes (with the range from 0 to 11.5 mL as listed below) through a linear transform, and the overall volume of a sample should be constrained to avoid overflow, which applies boundary conditions in the input space. After crossover and mutation, a new set of experiments will be generated through the reverse linear transform to map the variables back to the reagent volumes. The ranges of the volumes for individual reagent and boundary conditions are listed below:
CTAB (0.2 M): 0 to 11.50 mL
HAuCl₄ (1.0 mM): 0 to 11.50 mL
AgNO₃ (0.25 mM): 0 to 11.50 mL
Ascorbic acid (3.3 mM): 0 to 11.50 mL
Boundary conditions: Σᵢⁿvᵢ≤1 and vᵢ∈[0,1]

where vᵢ is the normalized variable from the linear transformation of the reagent volume (CTAB, HAuCl₄, AgNO₃ and ascorbic acid). Note in every experiment, an extra amount of water was added to keep the overall volume constant if necessary.

In the mutation, a vector will be generated from the multiple-dimensional Gaussian distribution and is then added to the original sampling point. If the addition breaks a boundary condition, an absorption boundary will be set: the perturbation vector will be scaled down so that it is still within the boundary (FIG. 29a-b). For samples on the boundaries, invalid mutations can happen due to the scaling operations and absorption boundary conditions described above, which can trap the samples there. To mitigate this problem, a flag is created according to the chance of mutation first and if the sample should mutate, mutation was performed until a valid outcome occurs (FIG. 29c).

In the crossover, after exchanging variables between two samples, the boundary condition can be broken. In this case, all the input variables will be scaled down so that the summation of them is 1 to maintain the boundary condition and avoid overflow in the experiments (FIG. 29d).

### Algorithm Parameters

The investigation of chemical space 1 was divided into three stages, each of which was subdivided into multiple steps. A single step of any stage was simply 24 reactions performed on the platform (including the standard sample). To begin, the first random sampling step and the subsequent 9 steps were for the open-ended exploration without bias, with multiple systems and general fitness functions (stage 1). TEM validation confirmed the existence of nanorods in multiple-peak systems when fitness was concerned with fitness scenario 1 (increasing the prominence of a single peak). The constrained exploration (stage 2) which focused on samples with multiple-peak features, with an explicit fitness function, was conducted for 4 steps. The new fitness function was defined so that the less prominent second peak was minimized. The final of the three stages was an exploitation process and was performed for an additional two steps. During exploitation, the absorbance signals from by-products were explicitly considered during the search. The function of this final stage was to decrease/remove signals from assumed by-products. The majority of samples with further increased absorption bands from the most prominent peaks were discovered after stage 2 and stage 3.

Among the 23 samples available per step of any given stage, 10 samples were mutated from the elite (parent) set, 10 samples were from crossover among elites each of which had a 40% chance of mutation and 3 samples were randomly generated in the input chemical space. This distributed equal resources for crossover and mutation processes in each step. As the dimensionality of the chemical space is high, the relatively small initial random dataset (23) has the potential to bias the exploration, therefore a small portion of random sampling was added in every step to reduce the initial bias. The standard deviations of the multi-dimensional Gaussian distribution were set to 0.05, 0.08, 0.08 for stage 1, 2 and 3 respectively. The slight increase of the standard deviation in stage 2 and 3 was used to get rid of any local maxima from the open-ended exploration. Considering the dimensionality of the space, we set 1 step for initial random sampling and 15 steps for investigation using MAP-Elites.

### Open-Ended Exploration of Chemical Space 1 (First 10 Steps Including Random Sampling)

The open-ended exploration of the chemical space was started by sampling 23 points randomly. These samples were then used to create an initial parent set.

### Classes and Fitness Functions

In the open-ended exploration of the real chemical space, both single and multiple peaks should be considered. For samples with one single peak, the class was purely dependent on the subregion which its peak was located in. For samples with multiple peaks, the most prominent two peaks were selected for further evaluation. In several literature reports, many Au nanostructures have intrinsically two comparable prominent peaks^{9,10}. But also maximizing one of the peaks can lead to a higher yield of one nanostructure if these peaks correspond to two different nanoparticles. As described above, for the multiple-peak systems, we evaluated a given sample's performance in two scenarios: if the UV-Vis absorption is purely from one peak (scenario 1, Eq. (25)) or from both peaks (scenario 2, Eq. (26)). It was necessary to include both scenarios in the exploration to enable diversity. In the multiple-peak systems, the UV-Vis peak positions were no longer the only criteria for classification and extra classes with varying fitness should be considered.

### Wavelength Region Discretization:

### Single Peak System:

400-600 nm at 25 nm intervals
600-950 nm at 50 nm intervals

### Multiple Peak System:

400-950 at 50 nm intervals.

In the single peak system, the class assigned is entirely derived from the subregion the peak lies in. In the multiple peak system, the subregion indexes in which the most and the second most prominent peaks are located were obtained, and used to determine the class. The two fitness functions were further evaluated which indicated if one of the peaks is dominant or both peaks contributed significantly to the spectrum respectively. Finally, this sample was assigned to two classes corresponding to the two fitness functions respectively. This strategy can create elites with similar peak positions but different UV-Vis features, which further enabled the diversity of our exploration.

The fitness functions (F) used in the single-peak system (Eq. (24)) and the two different scenarios of the multiple-peak system (Eq. (25) and (26)) are defined as follows:

### Single-Peak System:

$$F = \frac{{\int }_{{x}_{p e a k 1} - w}^{{x}_{p e a k 1} + w} {I}_{x} d x}{\int {I}_{x} d x} \qquad ( 24 )$$

### Multiple-Peak Systems:

Scenario 1: UV-Vis from One Dominant Peak:

$$F = \frac{{\int }_{{x}_{p e a k 1} - w}^{{x}_{p e a k 1} + w} {I}_{x} d x}{\int {I}_{x} d x} \qquad ( 25 )$$

Scenario 2: UV-Vis from Both Peaks:

$$F = \frac{{\int }_{x ∈ A ⋃ B} {I}_{x} d x}{\int {I}_{x} d x} \qquad ( 26 )$$

where Iₓ is the absorption of the UV-Vis spectrum at wavelength x, xₚₑₐₖ₁ and xₚₑₐₖ₂ are the position of the most and the second most prominent peaks, the integration range in Eq. (26) is defined by the union of two sets (A∪B), where A=[xₚₑₐₖ₁−w, xₚₑₐₖ₁+w] and B=[xₚₑₐₖ₂−w, xₚₑₐₖ₂+w], and w is a parameter to define the region near the peak and is tunable for individual fitness functions. Here we used 50 nm for all the functions in the exploration.

### Constrained Exploration and Exploitation in Chemical Space 1 (Steps 11-16, Including Stage 2 and 3)

Following from the initial 10 steps of exploration of chemical space 1, Au nanorods were found in the multiple-peak system when following scenario 1. Later, the criterion of classification was changed to put more focus on this scenario. During the initial steps of open-ended exploration, the focus of scenario 1 fitness function (Eq. (25)) was purposefully general. It was used to calculate the absorption area percentage near the dominant peak, however, this alone is not explicit enough to guide further search. A more explicit fitness function to decrease the secondary peak, decrease peak broadness and also to minimize the formation of by-products should be defined (Eq. (27)). The search continued for another 4 and 2 steps (stage 2 and 3 respectively) successively with different coefficients of the new fitness function (Eq. (27)). Overall, they correspond to the three stages with varying evaluation criteria for open-ended exploration (10 steps), constrained exploration (4 steps) and exploitation (2 steps).

The aim of constrained exploration and exploitation is to efficiently increase the original fitness value (Eq. (25)) by considering other explicit factors that influences the absorption band. The explicit features considered by (Eq. (27)) allowed us to increase not only fitness values in stages 2 and 3, but also the more general implicit fitness measure of Eq. (25) of the final individual elites. The original fitness function represents a general form of sample performance and the samples with the highest scores regarding to it were selected for TEM validation.

### Classes and Fitness

The class definition in both constrained exploration and exploitation is similar to that in the simulated chemical space. To make the exploration focused, the possible number of classes was decreased. The wavelength ranging from 400 nm to 950 nm was discretized with an interval of 50 nm and the most prominent must be in a higher wavelength subregion compared to the second one. Depending on which subregion the most prominent peak was in, a class index was assigned to it. The explicit fitness function (F) is defined below:

$$F = - {k}_{1} {w}_{1} - {k}_{2} {w}_{2} - {k}_{3} \overline{{I}_{x , w}} - {k}_{4} \overline{{I}_{b}} \qquad ( 27 )$$

where w₁ and w₂ are the widths at half prominence of the most and second most prominent peak, I̅_̅{̅x̅,̅w̅}̅ is the average absorption near the lower peak position x within a range of w, I̅_̅{̅b̅}̅ is the average absorption of a user-specified region that indicates the existence of by-products. Here this region was set to be from 550 nm to 600 nm and also w=50 nm.

To initialise stage 2, all the available data were evaluated with k₁=0.002, k₂=0, k₃=1, k₄=0 to create the initial parent set. The coefficients were selected to decrease the composition of the lower peak and minimize the broadness of the most prominent peak. 4 steps were run with the same coefficient for constrained exploration. A further 2 steps were run with modified coefficients of k₁=0.002, k₂=0.002, k₃=1, k₄=1, which defined the fitness more explicitly by considering the by-product peak to increase purity. In the later 2 steps, the search is more focused on minimizing the peaks corresponding to spheres or potential polyhedra, while the broadness of the most prominent peak should be maintained, so that the monodispersity of the rods were not sacrificed. After the constrained exploration and exploitation, all the available samples would be re-evaluated by the original fitness (Eq. (25)) for the final solutions. Consequently, four new nanorod samples with increased fitness were found. The UV-Vis and TEM characterisation will be discussed below.

### 3.2.3 Results and Discussion

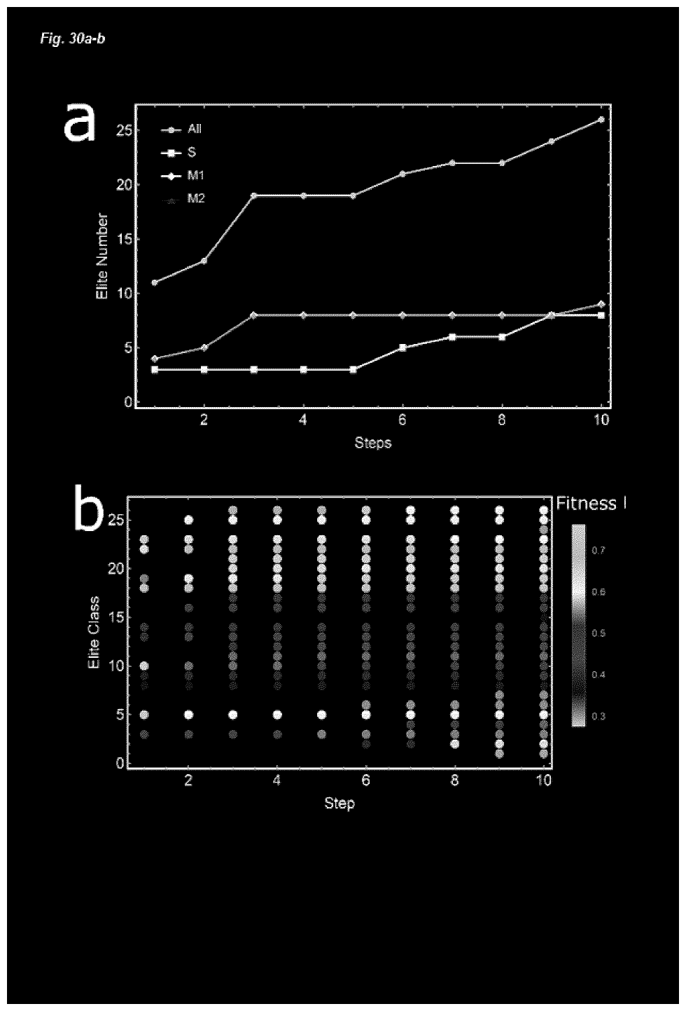

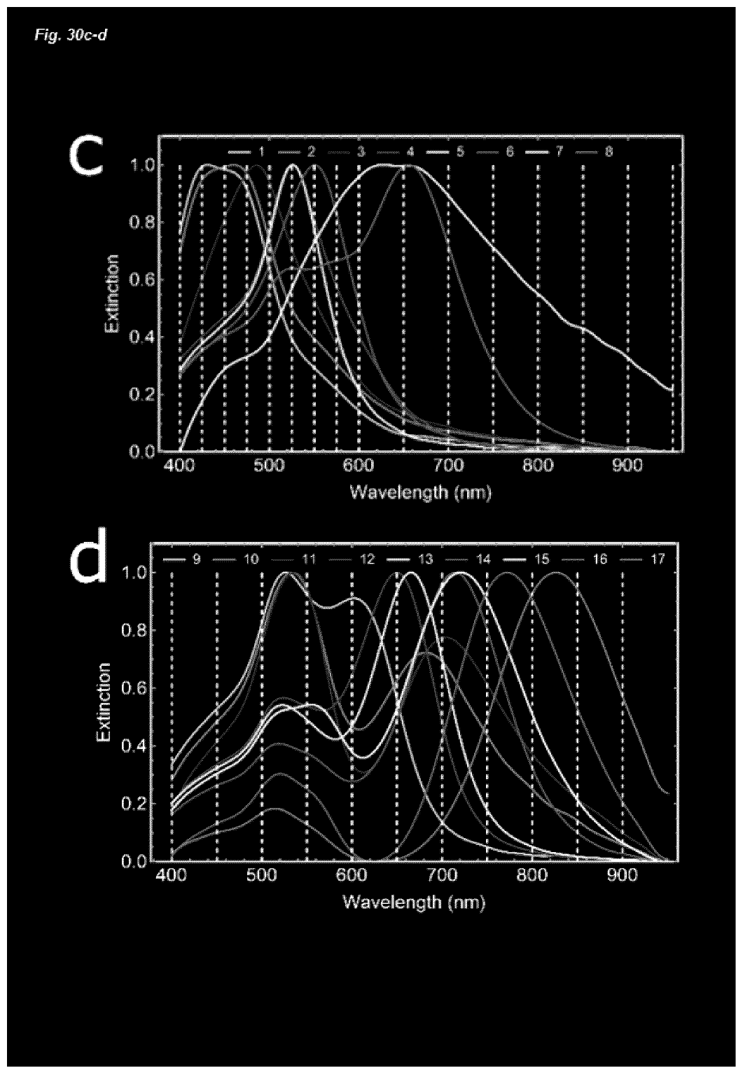

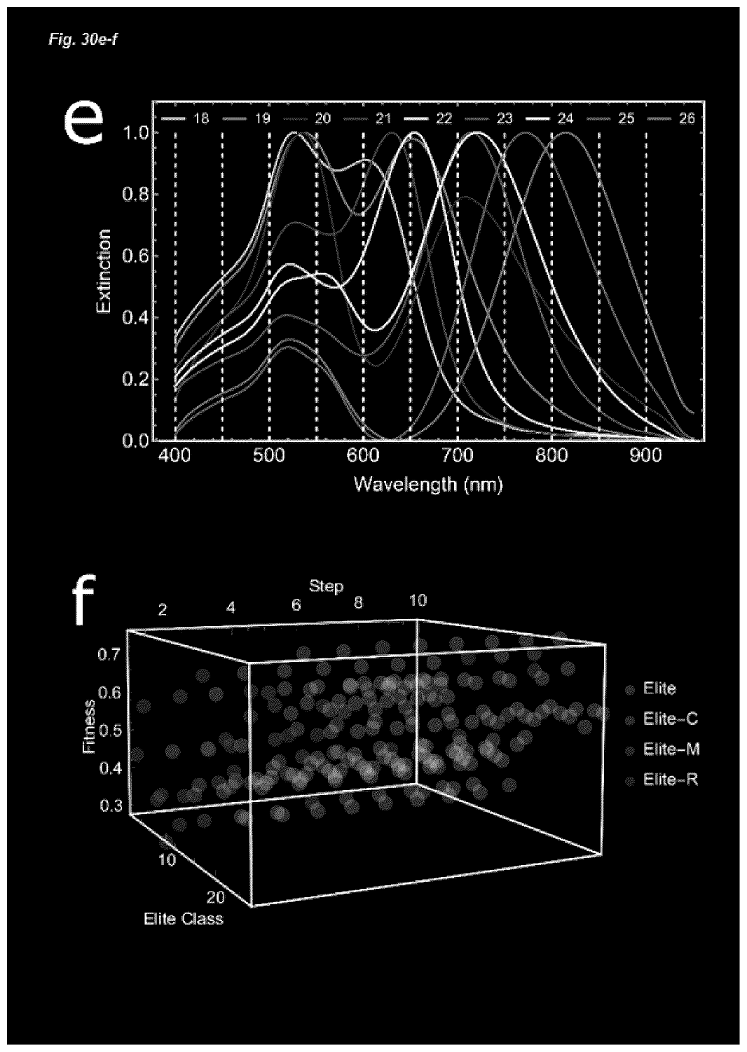

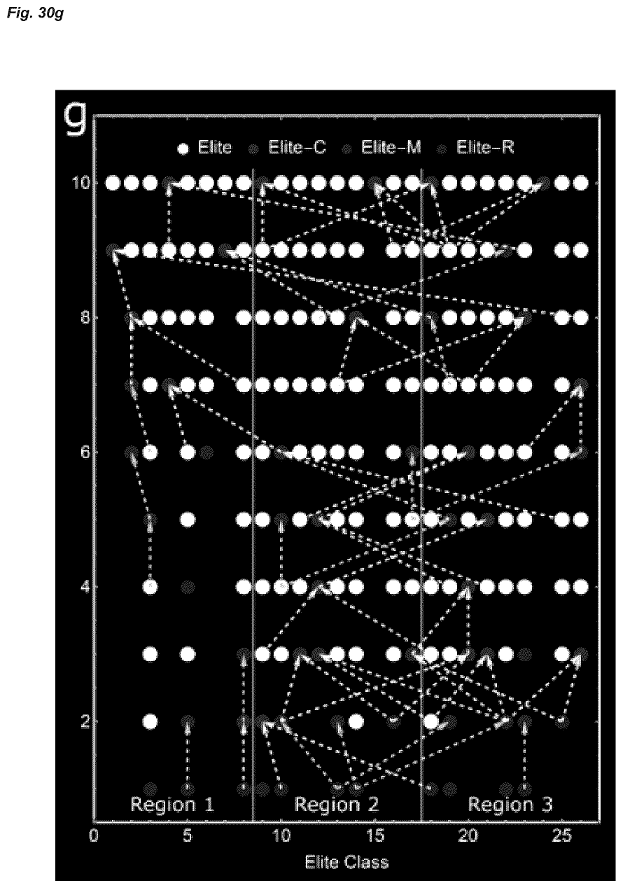

After the initial 10 steps of open-ended exploration, 26 elites were found (FIG. 30a). Depending on the peak numbers and the desired UV-Vis features, they were divided into three sets: 1. single-peak system; 2. multiple-peak system with scenario 1; 3. multiple-peak system with scenario 2. The final number of elites contributed from each set was 8, 9, and 9 respectively. The fitness values of different elites in the exploration are shown in FIG. 30b. The UV-Vis spectra of the individual sets are shown in FIG. 30c-e.

In the single-peak system, a variety of samples with their peak positions spreading from 420 nm to 650 nm were obtained.

For the multiple-peak systems with different scenarios, their elite numbers are the same in every step. The diversity of this system was extended so that the elites with similar peak positions can have different relative peak intensities (e.g., Elite 10 & 19 with peaks in 500-550 nm and 650-700 nm; 17 & 26 with peaks in 500-550 nm and 800-850 nm in FIG. 30) by defining the two search scenarios as described above. The synthetic conditions for the elites are listed in Table 3-2.

It is essential to analyse the emergence of the new elites in the open-ended exploration. The elites can be newly found or get replaced by samples with higher fitness in the same class. As shown in FIG. 30g, the new elites can be from the crossover, mutation or random sampling with increased fitness, indicating all the three sampling strategies contribute to generate new elites.

During the open-ended exploration, three sets were studied including 1. Single-peak system; 2. multiple-peak system with one peak contributing to the spectrum (scenario 1); 3. multiple-peak system with two peaks contributing to the spectrum (scenario 2). Thus, the elites can be divided into three sets, which are separated as region 1, 2 and 3 respectively in FIG. 30g. Region 1 belongs to the single-peak system while regions 2 and 3 belong to the multiple-peak systems. The times of generating new elites from previous elites of different systems define their interactions as discussed further below.

TABLE 3.2

| Class | CTAB | HAuCl₄ | AgNO₃ | Ascorbic acid | Water | Seed |
|---|---|---|---|---|---|---|
| Index | (mL) | (mL) | (mL) | (mL) | (mL) | (mL) |
| 1 | 0.90 | 6.46 | 3.56 | 0.12 | 0.46 | 0.50 |
| 2 | 0.89 | 6.22 | 3.10 | 0.00 | 1.29 | 0.50 |
| 3 | 0.06 | 6.02 | 3.21 | 0.00 | 2.21 | 0.50 |
| 4 | 1.12 | 1.72 | 0.09 | 0.79 | 7.78 | 0.50 |
| 5 (L1-1) | 1.51 | 2.53 | 0.09 | 7.13 | 0.24 | 0.50 |
| 6 | 1.62 | 1.05 | 2.52 | 5.54 | 0.77 | 0.50 |
| 7 | 0.00 | 3.31 | 0.53 | 2.75 | 4.91 | 0.50 |
| 8 | 2.70 | 1.74 | 0.41 | 6.05 | 0.60 | 0.50 |
| 9 | 0.78 | 3.28 | 0.29 | 4.31 | 2.84 | 0.50 |
| 10 | 0.78 | 1.72 | 3.29 | 0.79 | 4.92 | 0.50 |
| 11 | 1.31 | 1.72 | 3.29 | 0.79 | 4.39 | 0.50 |
| 12 | 1.31 | 4.42 | 0.48 | 2.88 | 2.41 | 0.50 |
| 13 | 0.65 | 3.71 | 0.48 | 1.84 | 4.82 | 0.50 |
| 14 | 1.12 | 3.57 | 0.67 | 1.64 | 4.50 | 0.50 |
| 15 | 3.47 | 2.72 | 2.33 | 2.58 | 0.40 | 0.50 |
| 16 | 5.23 | 1.72 | 3.29 | 0.79 | 0.47 | 0.50 |
| 17 (L1-6) | 4.86 | 2.74 | 2.93 | 0.97 | 0.00 | 0.50 |
| 18 | 0.78 | 3.28 | 0.29 | 4.31 | 2.84 | 0.50 |
| 19 | 0.78 | 3.28 | 1.83 | 2.49 | 3.12 | 0.50 |
| 20 | 1.31 | 1.72 | 3.29 | 0.79 | 4.39 | 0.50 |
| 21 | 0.95 | 4.89 | 0.46 | 4.15 | 1.05 | 0.50 |
| 22 | 1.03 | 5.01 | 0.49 | 2.51 | 2.46 | 0.50 |
| 23 | 1.12 | 3.57 | 0.67 | 1.64 | 4.50 | 0.50 |
| 24 | 3.47 | 2.72 | 2.33 | 2.58 | 0.40 | 0.50 |
| 25 | 5.23 | 1.72 | 3.29 | 0.79 | 0.47 | 0.50 |
| 26 | 1.58 | 3.30 | 2.93 | 0.97 | 2.72 | 0.50 |

The input parameters of the elites after exploring the chemical space 1 for 10 steps. The concentrations of the reagents are available in the same section above.

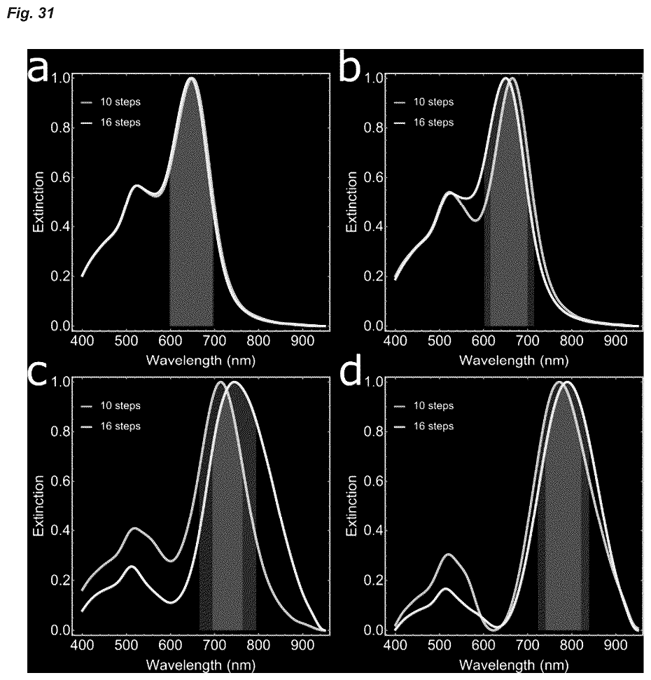

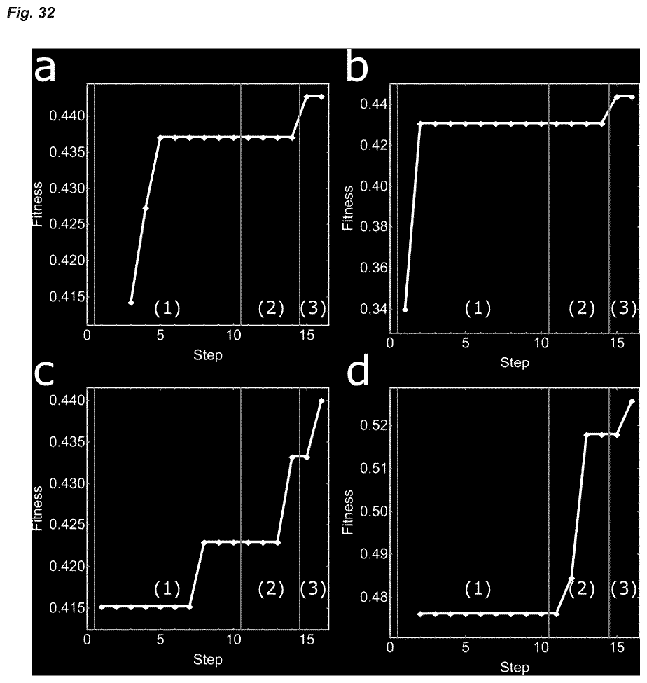

After the open-ended exploration, nanorods were discovered in the multiple-peak system (scenario 1). The UV-Vis peak with a longer wavelength corresponds to the longitudinal mode of nanorods, while the shorter one corresponds to the transverse mode, together with by-products of spheres, triangles or polyhedra. To increase the absorption band of the Au nanorods samples, further 4 steps for constrained exploration (stage 2) and 2 steps for exploitation (stage 3) were run with new classification criteria and fitness as described above. It is noted all the five elites corresponding to Au nanorods with different aspect ratios are available after the initial 10 steps of exploration (stage 1). Comparing to the results from the open-ended exploration, the further search found samples with increased absorption band (Eq. (25)). Consequently, four new Au nanorods (R1-R4) with higher scores were available. The UV-Vis spectra of the best samples before and after the two stages are shown in FIG. 31 for comparison. The increase of the absorption band defined by the original fitness function (Eq. (25)) of these four classes during the overall 16 steps are shown in FIG. 32.

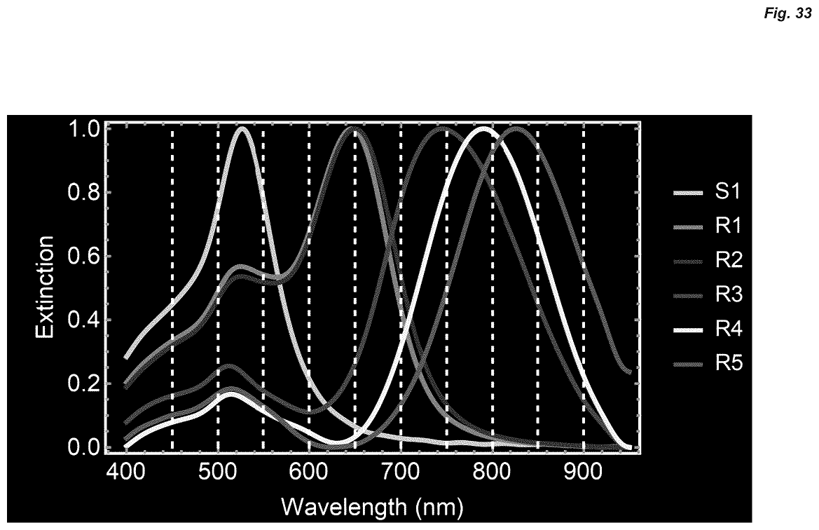

The final UV-Vis spectra of Au nanospheres and nanorods are shown in FIG. 33. The synthetic conditions for the nanorods as well as the nanospheres are shown in Table 3-3. Their labels in the manuscript were also shown in the same table. Note the two nanorod samples with low aspect ratios (R1 and R2) belong to different classes with peak positions near the boundary of the subregions. A similar phenomenon was also observed in the simulated chemical space (FIG. 18c).

TABLE 3-3

| | CTAB | HAuCl₄ | AgNO₃ | Ascorbic acid | Water | Seed |
|---|---|---|---|---|---|---|
| Label | (mL) | (mL) | (mL) | (mL) | (mL) | (mL) |
| S1 (L1-1) | 1.51 | 2.53 | 0.09 | 7.13 | 0.24 | 0.50 |
| R1 (L1-2) | 1.31 | 4.42 | 0.48 | 2.88 | 2.41 | 0.50 |
| R2 (L1-3) | 1.70 | 5.14 | 0.58 | 2.91 | 1.17 | 0.50 |
| R3 (L1-4) | 4.28 | 2.90 | 1.26 | 1.04 | 2.02 | 0.50 |
| R4 (L1-5) | 4.66 | 2.83 | 1.27 | 1.17 | 1.57 | 0.50 |
| R5 (L1-6) | 4.86 | 2.74 | 2.93 | 0.97 | 0.00 | 0.50 |

The input parameters of the final products in chemical space 1. The concentrations of the reagents are available in the same section above.

Nanospheres and nanorods with different aspect ratios were found.

Because the as-synthesised solutions would be used as the new seeds, to reflect the actual states of them, all samples for TEM characterisation were only centrifuged at 12,000 rpm for 10 minutes and washed by water twice to concentrate them, without any further purification.

The fourth rod sample (R4) with a dominant peak around 790 nm has a relatively high aspect ratio (compared to R1 and R2) with a concave and sharper contour compared to R3 and R5. It was used as the seed for the next level of exploration, assuming these features can lead to emergence of novel nanostructures. The chemical space using nanorods as the seed will be discussed in the next section.

### 3.3 Chemical Space 2: Overgrowth of Au Nanorods

A sample of Au nanorods was used as the seed for the further exploration in the second chemical space, with an extra pH variable to influence the growth kinetics. The Au nanorod seed (R4, also labelled as L1-5 in the manuscript) with concave features were reproduced and used directly as the seed solution. Hydroquinone was used as the reductant, whose redox potential is sensitive to the solution pH and can influence the growth kinetics. In chemical space 1, the strong interactions within the multiple-peak systems, but relatively weak interactions between the multiple-peak and single-peak systems were observed. To make the search focus on these two different systems respectively, the strategy was modified so that the exploration will only consider the multiple-peak systems first, then only the single system, which means the exploration happens sequentially.

### 3.3.1 Experimental Details

### Synthesis of Seed

Au nanorods (R4, also labelled as L1-5 herein) were reproduced with the same conditions from studying the first chemical space and aged in 30° C. for 1 hour to complete the growth. The solution was used within 24 hours.

### Synthetic Procedure

Each reaction was performed with the followed order of addition by the platform, using volumes and pH provided by the algorithm:
1. CTAB (0.2 M) added.
2. Hydroquinone (13.1 mM) added.
3. Water added to keep the overall volume as 7.00 mL.
4. pH measure and control of the solution using HCl (0.1 M) and NaOH (0.1 M).
5. AgNO₃ (0.25 mM) added.
6. HAuCl₄ (1.0 mM) added.
7. 10 seconds allowed for the reduction to complete.
8. 0.50 mL of the Au nanorod solution (R4) added.

The pH was tuned to a required value (step 4) before adding any metallic salts because the nanoparticles can form spontaneously without any seed in strong basic conditions and be adsorbed to the surface of the electrode, which can reduce the stability of the system. Before tuning the pH, the overall volume of the solution is constrained to a constant value, which can stabilize the liquid level and pH response. To enable the stability and reproducibility, the pH probe was calibrated daily with standard pH buffer solutions (4.0, 7.0 and 10.0). Before measuring the pH, the pH probe was immersed in the solution for 10 seconds to give enough time to reach the steady state.

In this chemical space, the pH was controlled with a proportional control logic with an overshoot mechanism for a given target pH (pH_{T}). It is described as following:
1. The initial current pH (pH_{C}) was measured and the difference between it and the target was calculated via Eq. (28).
2. Depending on if the target pH is higher or lower than the current pH (pH_{C}), NaOH (0.1 M) or HCl (0.1 M) was added respectively. The volume (V_{add}) for addition was calculated through the proportional control logic (Eq. (29)).
3. The current pH (pH_{C}) was measured again and to see if the solution pH overshot the target pH after the addition. If it overshot, the proportional coefficient k to calculate the next addition volume would be reduced by a factor (f) via Eq. (30).
4. If the termination condition is reached, stop the tuning. Otherwise, go back to step 2. There are three cases for the termination:
   a. The current pH was within a small range of the target pH (pH_{T}±0.2). The current pH (pH_{C}) would be updated after 15 seconds and would still stay in this range (success).
   b. The overall time to tune the pH value was more than 2 minutes, which enables efficiency (failure).
   c. The total volume of the added acid and base was more than 3 mL, which avoids overflow (failure).

$$ΔpH = {pH}_{T} - {pH}_{C} \qquad ( 28 )$$

$${V}_{a d d} = k \vert ΔpH \vert \qquad ( 29 )$$

$$k = f \times k \qquad ( 30 )$$

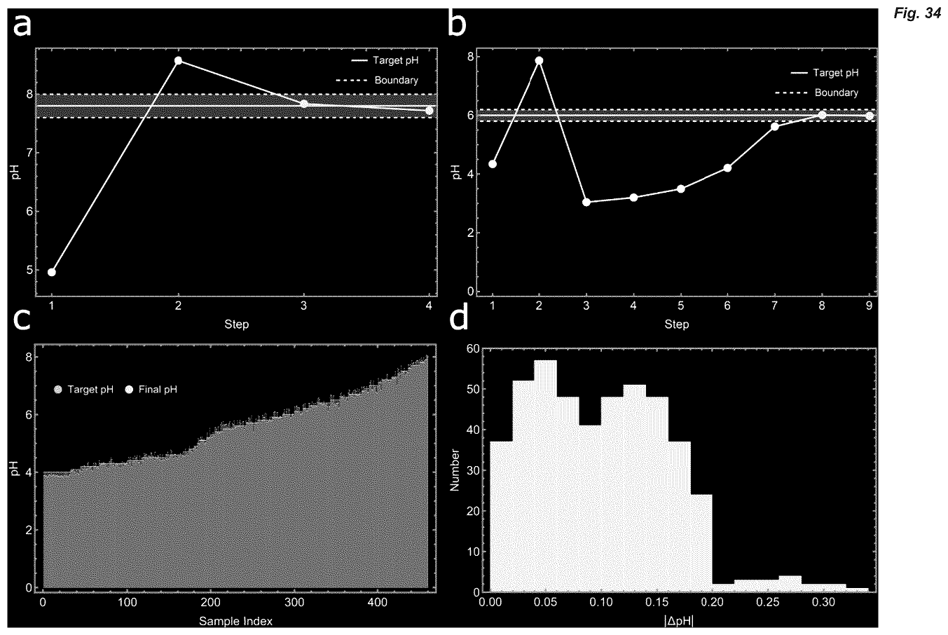

Step 2-4 was iterated until a termination condition is reached. We set the initial k to 25 μL and f to 0.6 in the experiment. The final actual pH was recorded and transformed to the input pH variable in the algorithms. Two test examples for the pH control are shown in FIGS. 34a and b. The target pHs and the final pHs in exploring chemical space 2 (460 samples in total) are shown in FIGS. 34c and d. The operations for the pH control in investigating chemical space 2 are recorded and used to reproduce the samples later.

### UV-Vis Characterisation and Data Processing:

As described above for Chemical Space 1.

### TEM

As described above for Chemical Space 1.

### Stability of the System:

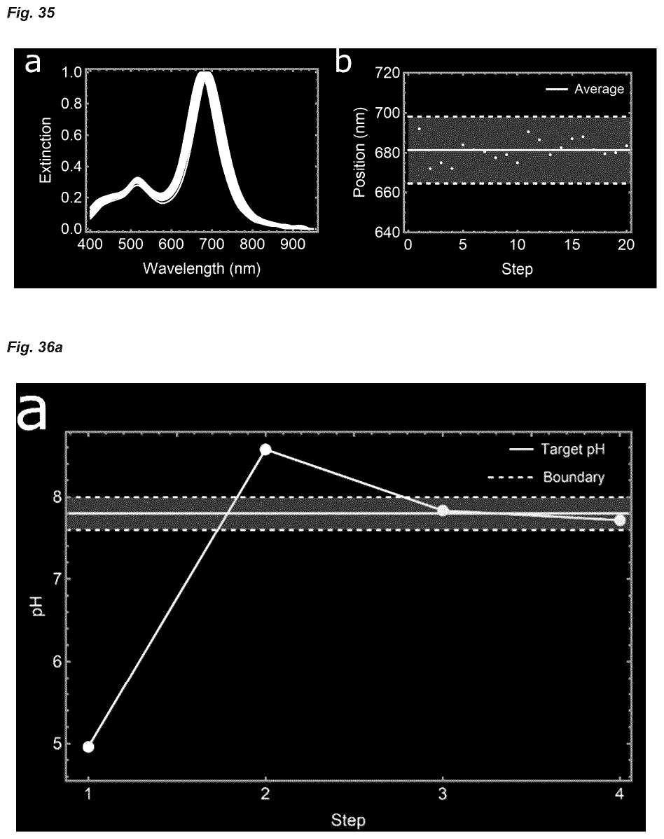

Similar to that in chemical space 1, samples with the same synthetic conditions were produced repeatedly throughout the exploration for each step. They served as standards to demonstrate the stability of the platform. Stock solutions can be reprepared during the repeats. The synthetic condition is listed in Table 3-4. The UV-Vis spectra of these standard samples were recorded to show the stability of the system (FIGS. 35a and b).

TABLE 3-4

| | | Hydro- | | | | | |
|---|---|---|---|---|---|---|---|
| | CTAB | quinone | Water | | AgNO₃ | HAuCl₄ | Seed |
| Standard | (mL) | (mL) | (mL) | pH | (mL) | (mL) | (mL) |
| 2 | 0.26 | 0.20 | 6.54 | 6.34 | 1.28 | 2.02 | 0.50 |

The input parameters as the standard to test the stability of the autonomous platform in chemical space 2. The concentrations of the reagents are available in the same section above.

### 3.2.2 Data Processing and Algorithm

### Input Space, Boundary Conditions and Sampling Strategy

A linear transformation was applied to transform between volumes of chemicals and variables in the algorithm. These variables are always normalized between 0 and 1. The volume range for individual chemicals and pH are listed below:
CTAB (0.2 M): 0 to 7.00 mL
Hydroquinone (3.3 mM): 0 to 7.00 mL
HAuCl₄ (1.0 mM): 0 to 5.00 mL
AgNO₃ (0.25 mM): 0 to 5.00 mL
Target pH: 4.0 to 8.0
Boundary conditions: v_{CTAB}+v_{HQ}≤1 and vᵢ∈[0,1]

where vᵢ is the normalized variable from a linear transformation from the volume of reagent i and also the solution pH. v_{HQ} means the variable from the volume of hydroquinone. Note that the boundary condition was put so that the summation of v_{CTAB} and v_{HQ} is no larger than 1. In generating new experiments, the crossover, mutation and random sampling are the same as these discussed in Section 3.2.

### Algorithm Parameters

Among the 23 samples available per step, 10 samples were mutated from the elite (parent) set, 10 samples were from crossover among elites each of which had a 40% chance of mutation and 3 samples were randomly generated in the input chemical space. This distributed equal resources for crossover and mutation processes in each step. As the dimensionality of the chemical space is high, the relatively small initial random dataset (23) has the potential to bias the exploration, therefore a small portion of random sampling was added in every step to reduce the risk of bias. The standard deviations of the multi-dimensional Gaussian distribution were set to 0.08.

### Exploration of the Chemical Space

The chemical space was explored by running 10 steps only considering the multiple-peak systems (with a random sampling number of 23 in the first step) and another 10 steps only considering the single-peak system. The exploration of the single-peak system was initialised with the data from the first 10 step exploration of the multiple-peak systems.

### Classes and Fitness

The classes were determined following the same procedure described for the single-peak system in the exploration of chemical space 1. For both single-peak and multiple-peak systems, from 400 nm to 600 nm the discretization was done with an interval of 25 nm and 600 nm to 950 nm with an interval of 50 nm.

The fitness (F) was defined in a similar way by considering the absorption band of the individual peak and its corresponding peak width. The fitness functions should guide multiple directions of exploration including:
1. For the single-peak system, the single peak should be sharp and dominant.
2. For the multiple-peak systems, the search should be making one of the peaks more dominant over the others (scenario 1), or two peaks comparable (scenario 2).

All peaks should be sharp to enable the monodispersity and purity of nanostructure populations. Based on these considerations, the fitness functions were defined as follows:

### Single-Peak System:

$$F = {k}_{1} \frac{{\int }_{{x}_{p e a k 1} - w}^{{x}_{p e a k 1} + w} {I}_{x} d x}{\int {I}_{x} d x} - {k}_{2} {w}_{1} \qquad ( 31 )$$

where k₁ and k₂ are coefficients to tune the importance of the individual terms. xₚₑₐₖ₁ is the peak position in the single-peak system and w defines the range of the absorption band and was set to 50 nm. w₁ is the peak width at its half prominence. We set k₁=1 and k₂=0.002 due to the scale of the two terms in the experiments.

### Multiple-Peak Systems:

$$F = {k}_{1} \vert \frac{{\int }_{{x}_{peak 1} - w}^{{x}_{peak 1} + w} {I}_{x} dx}{\int {I}_{x} dx} - \frac{{\int }_{{x}_{peak 2} - w}^{{x}_{peak 2} + w} {I}_{x} dx}{\int {I}_{x} dx} \vert - {k}_{2} ( {w}_{1} + {w}_{2} ) \qquad ( 32 )$$

where xₚₑₐₖ₁ and xₚₑₐₖ₂ are the peak positions of the most prominent two peaks, w defines the absorption band near them and was set to 50 nm, w₁ and w₂ are the peak widths at half prominence for the peaks respectively and k₁ and k₂ are coefficients controlling the importance of individual terms. The first term measures the difference between two peaks' absorption bands. Considering the scales and the relative importance of the two terms, we set k₁=1 and k₂=0.002 to amplify the difference in scenario 1, and k₁=−1 and k₂=0.002 to minimize the difference between absorption bands in scenario 2.

### 3.3.3 Results and Discussion

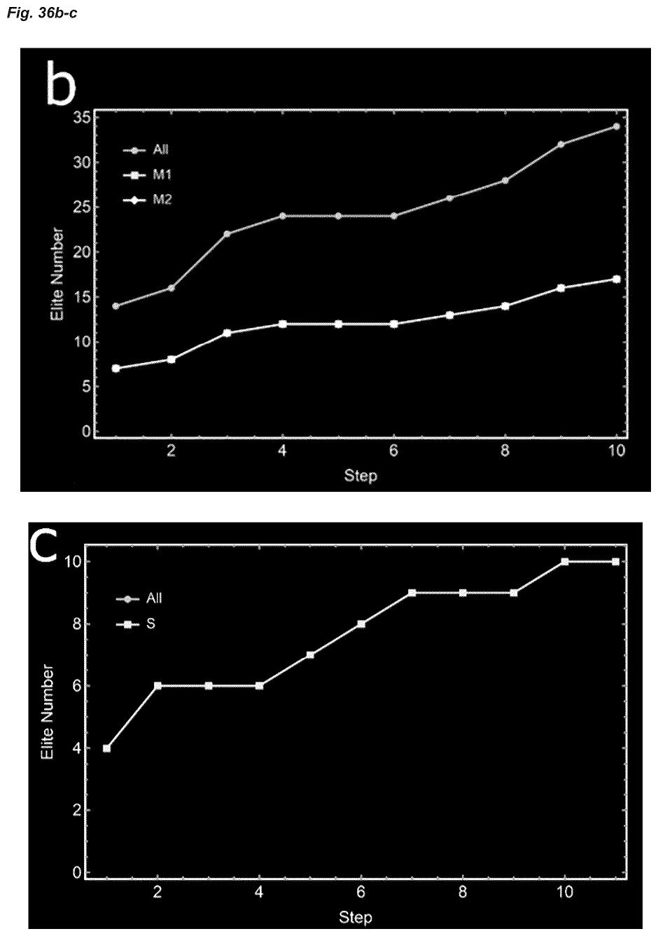

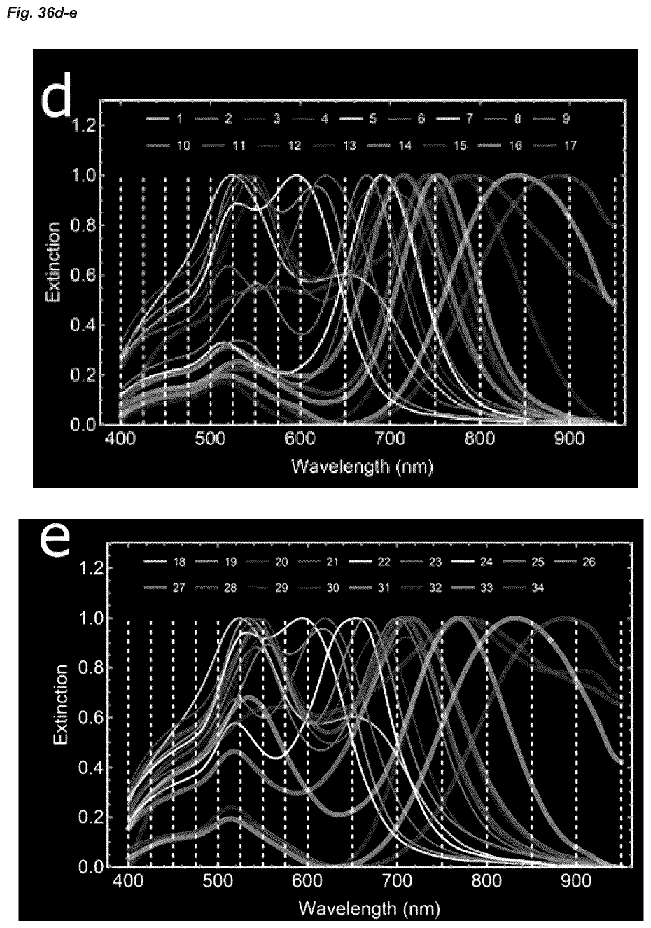

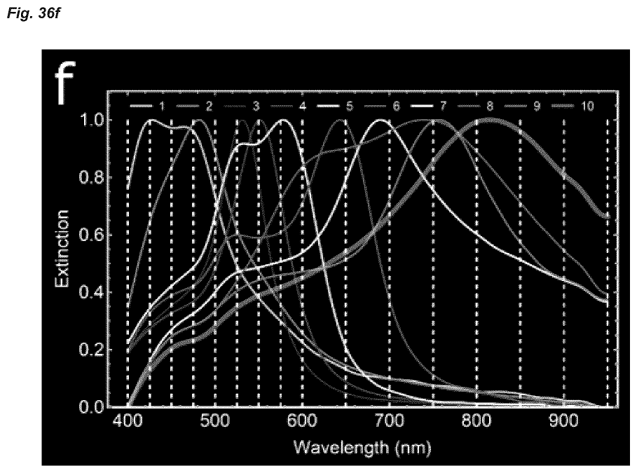

In chemical space 2, the pH-controlled overgrowth of Au nanorods was investigated (FIG. 36). The pH value was tuned to influence the growth kinetics via a proportional control logic (FIG. 36a) as discussed above. In chemical space 1, the interactions between the single-peak system and multiple-peak systems of either scenario, were weaker than those between the different scenarios of the multiple-peak systems, see the Section “Chemical space 1: Seed-mediated synthesis on cuboctahedron single crystals” below. The exploration was split into two stages:
1. Stage 1 consisted of 10 steps, in which the systems explored were only the multiple-peak systems with two different scenarios.
2. Stage 2 consisted of 10 steps also, in which the system explored was only the single-peak system. These 10 steps were initialised using all available data from stage 1.

Different elites were defined in these two stages respectively. The numbers of the elites belonging to multiple-peak systems in the first stage and the single-peak system in the second stage are shown in FIGS. 36b and c. The elites' UV-Vis spectra from both the multiple-peak systems, with two different scenarios, and the single-peak system are shown in FIG. 36d-f.

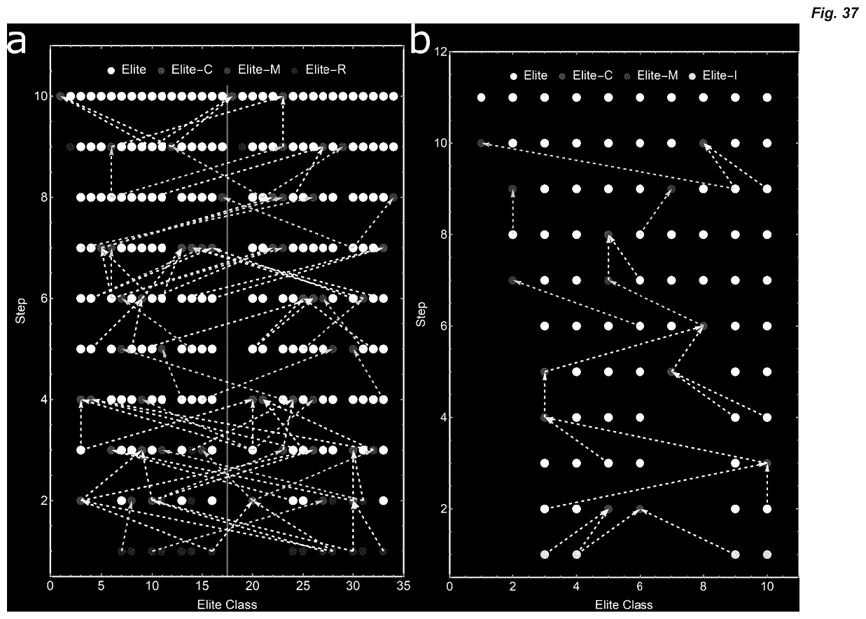

Peak positions spreading from 500 nm to 900 nm were found in the exploration of the multiple-peak systems. The two scenarios that were explored discovered samples with similar peak positions however, the relative intensities of the peaks from one elite to another varied significantly (See Elite 9 & 26 with peaks ranges in 550-575 nm and 650-700 nm; 10 & 27 with peak ranges in 500-525 nm and 700-750 nm as examples for comparison). The multiple-peak systems with different scenarios showed a strong interaction with crossover and mutation among elites to increase their fitness (FIG. 37a). In the multiple-peak systems, the Au nanorod seed was found to transform into three types of nanorods with different aspect ratios including: 1. nanorods with spherical caps (Elite 6, 7 and 22, which correspond to L2-1, L2-2 and L2-10 in the manuscript respectively); 2. nanorods with rectangular caps (Elite 8, 10, 13 and 17, which correspond to L2-3, L2-5, L2-7 and L2-9 in the manuscript respectively); 3. Irregular nanorods similar to dog bones (Elite 9, 11 and 14, which correspond to L2-4, L2-6 and L2-8 in the manuscript respectively). The synthetic conditions for the nanoparticles in the multiple-peak systems are listed in Table 3-5.

The single-peak system was initialised utilising the data from stage 1. The initial elite number was 4 and it increased to 10 after stage 2. Stage 2 exploration was purely driven by the crossover and mutation of the elites as shown in FIG. 37b. In the single-peak system, the peak position can vary from 400 nm to 820 nm and the goal of the fitness was to make this peak more dominant. Samples of elites 6-10 showed strong aggregation and/or overreduction to bulk gold, which would result in a cloudy solution with a large, broadened UV-Vis peak. Elites 1 and 2 produced peak positions below 525 nm were found where a very small volume reductant was added to reduce HAuCl₄. We characterised the remaining elites and found uniquely-shaped Au NPs of spherical polyhedra, bicones and low aspect-ratioed rods, with the spherical polyhedra and bicones transforming into smooth spheres after leaving them overnight (˜ 16 hours). Then the Au nanospheres from bicones with a peak position around 530 nm was used as the seed for the further exploration (chemical space 3), which will be discussed in detail in the next section. The synthetic conditions for the elites found in the single-peak system are shown in Table 3-6.

TABLE 3-6

| | CTAB | Hydroquinone | Water | | AgNO₃ | HAuCl₄ | Seed |
|---|---|---|---|---|---|---|---|
| Class Index | (mL) | (mL) | (mL) | pH | (mL) | (mL) | (mL) |
| 1 | 0.07 | 0.24 | 6.69 | 5.91 | 1.29 | 2.76 | 0.50 |
| 2 | 0.09 | 0.00 | 6.91 | 6.91 | 1.31 | 2.85 | 0.50 |
| 3 (L2-11-1) | 2.95 | 1.15 | 2.90 | 4.30 | 0.00 | 2.65 | 0.50 |
| 4 (L2-12-1) | 1.85 | 1.07 | 4.08 | 6.68 | 0.07 | 3.09 | 0.50 |
| 5 (L2-13) | 0.17 | 0.51 | 6.32 | 6.45 | 0.84 | 3.07 | 0.50 |
| 6 | 0.01 | 0.34 | 6.65 | 6.38 | 1.32 | 2.65 | 0.50 |
| 7 | 0.00 | 0.35 | 6.65 | 6.40 | 1.85 | 2.16 | 0.50 |
| 8 | 0.00 | 1.20 | 5.80 | 4.32 | 0.00 | 3.22 | 0.50 |
| 9 | 0.00 | 0.76 | 6.24 | 5.78 | 1.78 | 3.22 | 0.50 |
| 10 | 0.00 | 1.20 | 5.80 | 4.25 | 0.00 | 2.65 | 0.50 |
| L2-11-2 | 2.95 | 1.15 | 2.90 | 4.30 | 0.00 | 2.65 | 0.50 |
| L2-12-2 | 1.85 | 1.07 | 4.08 | 6.68 | 0.07 | 3.09 | 0.50 |

The input parameters of the elites in the chemical space 2 after exploring the single-peak system. The concentrations of the reagents are available in the same section above. The pH is the experimental value. Note leaving L2-11-1 and L2-12-1 for 16 hours, they converted to L2-11-2 and L2-12-2 respectively.

Note Elite 5 has one dominant peak and a small shoulder peak which is under the threshold to be regarded as a separate peak. The sample is composed of low aspect-ratio nanorods with a longitudinal peak around 575 nm.

### 3.4 Chemical Space 3: Overgrowth of Au Nanospheres

The seed of the third chemical space is the Au nanosphere sample (L2-12-2) from chemical space 2. The five-dimensional input chemical space was defined by volumes of hexadecyl-trimethylammonium chloride (CTAC), AgNO₃, HAuCl₄, ascorbic acid and HCl. As a result, we managed to synthesise Au nanostars with different size and tip length.

### 3.4.1 Experimental Details

### Synthesis of the Seed

Au spheres (L2-12-2) were reproduced using R4 (as labelled as L1-5 in the manuscript) as the seed with the same conditions in the second chemical space by leaving it at 30° C. to grow around 16 hours.

### Synthetic Procedure

Each reaction was performed with the following order of addition by the platform, using volumes provided by the algorithm:
1. CTAC (0.2 M) added.
2. HAuCl₄ (1.0 mM) added.
3. AgNO₃ (0.25 mM) added.
4. HCl (0.1 M) added.
5. Ascorbic acid (13.1 mM) added.
6. 10 seconds allowed for the reduction to complete.
7. Total volume constrained by addition of water if necessary.
8. 0.50 mL of the Au nanospheres solution (L2-12-2) as prepared in the second chemical space added.

The overall volume of the synthesised sample was constrained to 12.00 mL by adding Type I water, while the seed solution volume was kept as 0.50 mL. Note the concentration of ascorbic acid was around four times as that in chemical space 1.

### UV-Vis Characterisation and Data Processing:

As described above for Chemical Space 1.

### TEM

As described above for Chemical Space 1.

### Stability of the System:

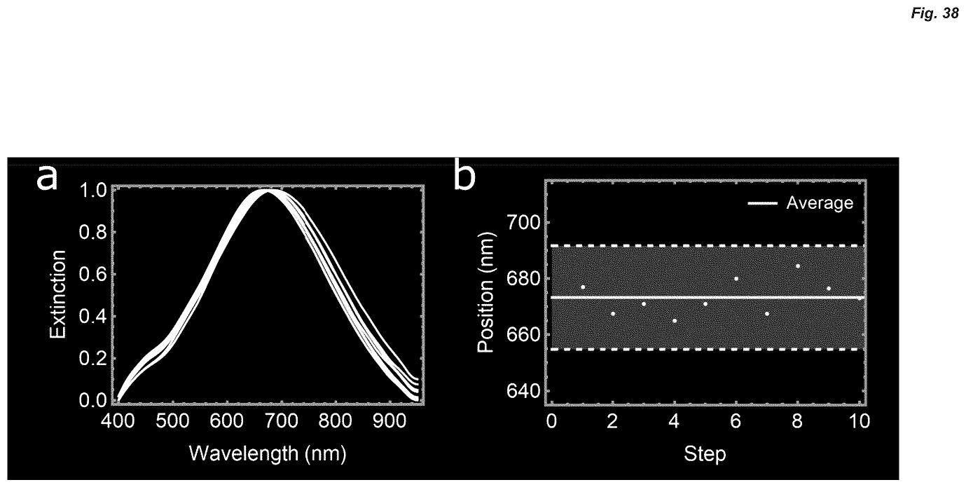

The standard samples were set to track the stability of the system. Unlike chemical space 1 and 2, no standard was set in the first step of random sampling due to the lack of knowledge of this chemical space. The stability of the system from the second step onwards were checked by selecting one sample from the first step as the standard sample. The UV-Vis spectra are shown in FIG. 38. The synthetic condition for the standard sample is listed in Table 3-7.

TABLE 3-7

| Stan- | CTAC | HAuCl₄ | AgNO₃ | HCl | Ascorbic | Water | Seed |
|---|---|---|---|---|---|---|---|
| dard | (mL) | (mL) | (mL) | (mL) | acid (mL) | (mL) | (mL) |
| 3 | 1.57 | 2.86 | 1.37 | 0.60 | 5.00 | 0.10 | 0.50 |

The input parameters as the standard to test the stability of the autonomous platform in chemical space 3. The concentrations of the reagents are available in the same section above. Note in chemical space 3, the concentration of ascorbic acid was around four times as that in chemical space 1.

### 3.4.2 Data Processing and Algorithm

The settings in chemical space 3 is almost the same as those in chemical space 2 with only modifications on the initial sampling number and classes which will be discussed below.

### Input Space, Boundary Conditions and Sampling Strategy

As in chemical space 1, a linear transformation is applied for the conversion between volumes of chemicals and variables in the algorithm. These variables are always normalized between 0 and 1. The volume range for individual chemicals and boundary conditions are listed below:
CTAC (0.2 M): 0 to 11.50 mL
HAuCl₄ (1.0 mM): 0 to 11.50 mL
AgNO₃ (0.25 mM): 0 to 11.50 mL
Ascorbic acid (13.1 mM): 0 to 11.50 mL
HCl (0.1 M): 0 to 11.50 mL
Boundary conditions: Σᵢⁿvᵢ≤1 for vᵢ∈[0,1]

where vᵢ is the normalized variable from a linear transformation from the volume of reagent i (CTAC, HAuCl₄, AgNO₃, ascorbic acid and HCl). The volume of the seed solution is fixed at 0.50 mL and extra water solution is added to constrain the overall volume (including seed solution) to be 12.00 mL.

### Algorithm Parameters

The initial random sampling number was 24 with no standard sample in the first step. The parameters for crossover, mutation and random sampling are the same as those in the chemical space 2.

### Exploration of the Chemical Space

Only the single-peak system was explored by 10 steps including the initial first random sampling step.

### Classes and Fitness

Only the single-peak system in this chemical space was explored. The classes were defined according to the subregion the single peak is located in, which is similar to the procedure described above in chemical space 2. The subregions were determined by putting 400 nm to 550 nm as a single subregion, discretizing 550 nm to 800 nm with an interval of 25 nm, and 800 nm to 950 nm with an interval of 50 nm respectively. This setting put more resources in exploring samples with a single peak in the range between 550 to 800 nm. The reason for choosing this approach is simply that the starting seed began with a signal at 530 nm, and therefore the overgrowth of this shape is most likely to exist within this range.

The fitness function is defined via Eq. (31) by setting w=50 nm, k₁=1 and k₂=0.002 respectively.

### 3.4.3 Results and Discussions

The results of exploring this chemical space are shown in FIG. 39. By enabling the diversity of UV-Vis spectra, Au nanostars with different sizes and tip length were found. The UV-Vis peak positions spread from 560 nm to 800 nm. The total elite number at the conclusion of this exploration was 11. The synthetic conditions for the elites are listed in Table 3-8. Elite 1 was found where almost no reductant (0.06 mL) was added, thus it was not characterised by TEM. The rest of the elites were characterised by TEM. Among them, Elite 2 and 3 showed a tendency to create octahedral features, and a small portion of Au octahedra were observed in Elite 3. These octahedral features will be further facilitated in the later optimisation discussed in next section.

TABLE 3-8

| Class | CTAC | HAuCl₄ | AgNO₃ | HCl | Ascorbic | Water | Seed |
|---|---|---|---|---|---|---|---|
| Index | (mL) | (mL) | (mL) | (mL) | acid (mL) | (mL) | (mL) |
| 1 | 0.04 | 6.27 | 3.46 | 0.98 | 0.06 | 0.69 | 0.50 |
| 2 (L3-1) | 2.66 | 0.32 | 0.00 | 0.74 | 5.25 | 2.53 | 0.50 |
| 3 | 0.21 | 0.29 | 0.42 | 2.18 | 3.82 | 4.58 | 0.50 |
| 4 | 2.24 | 1.54 | 4.01 | 2.18 | 0.33 | 1.20 | 0.50 |
| 5 | 2.24 | 1.54 | 0.02 | 2.18 | 3.82 | 1.70 | 0.50 |
| 6 (L3-2) | 0.37 | 1.04 | 0.11 | 7.02 | 0.48 | 2.48 | 0.50 |
| 7 (L3-3) | 6.81 | 1.92 | 0.03 | 1.19 | 0.60 | 0.95 | 0.50 |
| 8 (L3-4) | 1.10 | 1.39 | 0.27 | 6.50 | 1.10 | 1.14 | 0.50 |
| 9 | 5.09 | 1.29 | 0.19 | 4.52 | 0.41 | 0.00 | 0.50 |
| 10 (L3-5) | 3.78 | 2.49 | 0.80 | 3.58 | 0.54 | 0.31 | 0.50 |
| 11 | 7.72 | 2.24 | 0.12 | 0.28 | 1.14 | 0.00 | 0.50 |

The input parameters of the elites after exploration in chemical space 3. Note in chemical space 3, the concentration of ascorbic acid was around 4 times as that in chemical space 1.

### 3.5 Estimating the Possible Experiments in Exploration

The total number of possible experiments in exploring the three hierarchically-linked chemical space was estimated. We assume for a thorough grid search, one hundred different values for the volume of one chemical reagent are necessary. Regarding the pH variable, it is limited by the control decimal with an error tolerance of ±0.2. Considering the pH range from 4 to 8 we used, the possible different values of pH variable can be estimated as

$$\frac{8 - 4}{0.2} = 20 .$$

In calculating the possible combination number, the boundary condition we put to constrain the overall volume should be considered as well.

1. In chemical space 1, the freedom of the variables is four. Four reagent volumes (CTAB, HAuCl₄, AgNO₃ and ascorbic acid) can change freely with a linear constrain so that their total volume should be no larger than 11.5 mL. Thus, the number of the total combinations is

$${( {10}^{2} )}^{4} \times \frac{1}{2} \times \frac{1}{3} \times \frac{1}{4} ,$$

where

$$\frac{1}{2} \times \frac{1}{3} \times \frac{1}{4}$$

considers the influence from the constrain. In this estimation, the minimal volume interval is

$$\frac{11.5}{100} = 0.115 mL .$$

2. In chemical space 2, the freedom of the variables is five. Four reagent volumes (CTAB, HAuCl₄, AgNO₃ and hydroquinone) and the pH variable can change. The linear constrain is that the total volume of CTAB and hydroquinone should be no larger than 7.0 mL. Thus, the number of the total combinations is

$${( {10}^{2} )}^{4} \times 20 \times \frac{1}{2} ,$$

where ½ considers the influence from the constrain. In this estimation, the minimal volume interval is

$$\frac{5}{100} = 0.05 mL .$$

3. In chemical space 3, the freedom of the variables is five. Five reagent volumes (CTAB, HAuCl₄, AgNO₃, ascorbic acid and HCl) can change. The linear constrain is that the total volume of these five reagents should be no larger than 11.5 mL. Thus, the number of the total combinations is

$${( {10}^{2} )}^{5} \times \frac{1}{2} \times \frac{1}{3} \times \frac{1}{4} \times \frac{1}{5} ,$$

where

$$\frac{1}{2} \times \frac{1}{3} \times \frac{1}{4} \times \frac{1}{5}$$

considers the influence from the constrain. In this estimation, the minimal volume interval is

$$\frac{11.5}{100} = 0.1 \; 15 mL .$$

Since the chemical spaces were hierarchically-linked and any solution (regardless their morphology and polydispersity) in principle can be used as the seed, the number of the total possible combinations in the exploration is

$${( {10}^{2} )}^{4} \times \frac{1}{2} \times \frac{1}{3} \times \frac{1}{4} \times {( 1 {0}^{2} )}^{4} \times 20 \times \frac{1}{2} \times {( 1 {0}^{2} )}^{5} \times \frac{1}{2} \times \frac{1}{3} \times \frac{1}{4} \times \frac{1}{5} \approx 3.5 \times 1 {0}^{23} .$$

### 3.6 Mutation, Crossover and Random Sampling in the Exploration Algorithm

### MAP-Elites Serves Two Purposes in Exploration:

Facilitated the diversity of the samples and find new elites (classes) that did not exist before. Improve the performance of the existing elites.

During our experimental implementation, they are realised by three operations including crossover, mutation and random sampling. Crossover and mutation are evolutionary operations mimicking the natural evolution process. Compared to random sampling, these operations constrained the sampling points either near the current elites or as a combination of the input variables from the elites, while random sampling can search the space uniformly/without preference.

When the desired classes are distributed uniformly in the space, all three operations help to find the new elites. When crossover/mutation trapped the exploration due to their sampling constraint, random sampling help to add new input features. This is demonstrated by the observations of obtaining new classes through random sampling during the various exploration processes.

However, if the desired classes are within small subregions in the input space, replying on random sampling is inefficient. In this case, crossover/mutation, which maintain certain input features from the existing elites, help to target at these subregions efficiently. It is demonstrated by the exploration of the single-peak system in chemical space 2, where the new classes (Elite 6-8) emerged in very low concentration of CTAB. This feature is passed from the parent set via evolutionary operations and not easily available by random sampling.

To improve the performance of existing elites, the evolutionary operations played a dominant role over random sampling. The times of finding a better elite from evolutionary operations and random sampling in the open-ended exploration in the three chemical spaces are listed in Table 3-9.

TABLE 3-9
The times of improving the existing elite's performance
between evolutionary operations and random sampling in
the open-ended exploration of three chemical spaces.
Note the exploration process for multiple-peak and
single-peak systems in chemical space 2 are shown separately.

| | Chemical | Chemical | Chemical | |
|---|---|---|---|---|
| | | Space 2 | Space 2 | Chemical |
| Space | Space 1 | (Multiple-peak) | (Single-peak) | Space 3 |
| Ratio | 30:2 | 35:5 | 8:0 | 15:0 |

Considering the frequency of evolutionary operations and the random sampling in designing experiments (which is 20:3), the evolutionary operations are more likely to improve the performances of the elites. More importantly, it was at the early stage of the exploration that random sampling worked. When the performances of the elites were moderately good, improving the performances further with random sampling was unlikely to happen, which was consistent with the observation in benchmarking the algorithm in Section 2.5.

### 3.7 Monodispersity Analysis During Exploration

In the previous sections, TEM images for the discovered nanostructures showed their optimal monodispersity. Here we quantitively analysed the monodispersity of the discovered nanospheres and nanorods to give a performance metric of the results. The diameter of the nanospheres, and the width, length, and aspect ratio of the nanorods were analysed using their TEM images with at least 100 nanoparticles (see below). The polydispersity index (PDI) of the measured geometric parameter was also calculated via Eq. (33):

$$PDI ( x ) = {( \frac{{σ}_{x}}{E [ x ]} )}^{2} \qquad ( 33 )$$

where is the geometric parameter, E[x] is its average value from the measurement and ox the standard deviation.

### Chemical Space 1

### Nanospheres (L1-1):

The average size of the nanospheres (L1-1) was estimated by measuring the diameters of 500 nanospheres from the TEM images of a smaple. The mean and standard deviation of the diameters were 14.77 nm and 1.36 nm, respectively. The PDI from the measured diameters was calculated as 0.008.

### Nanorods (L1-2 to L1-6):

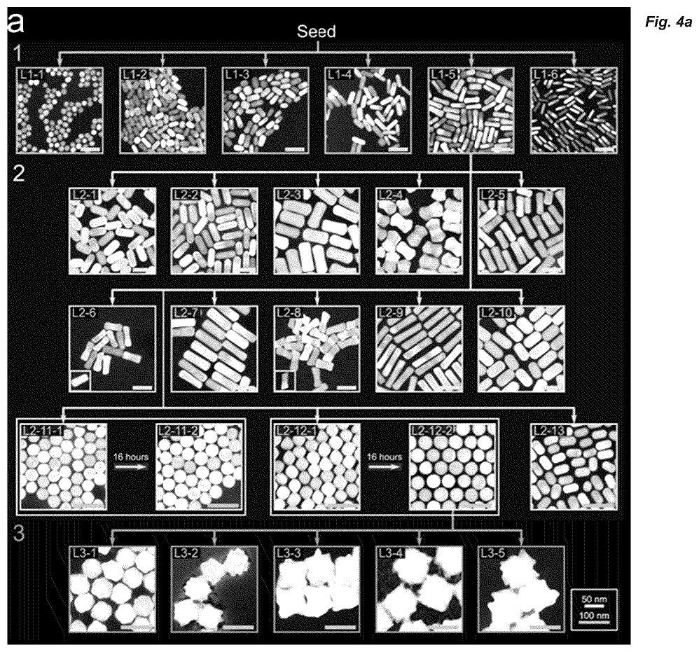

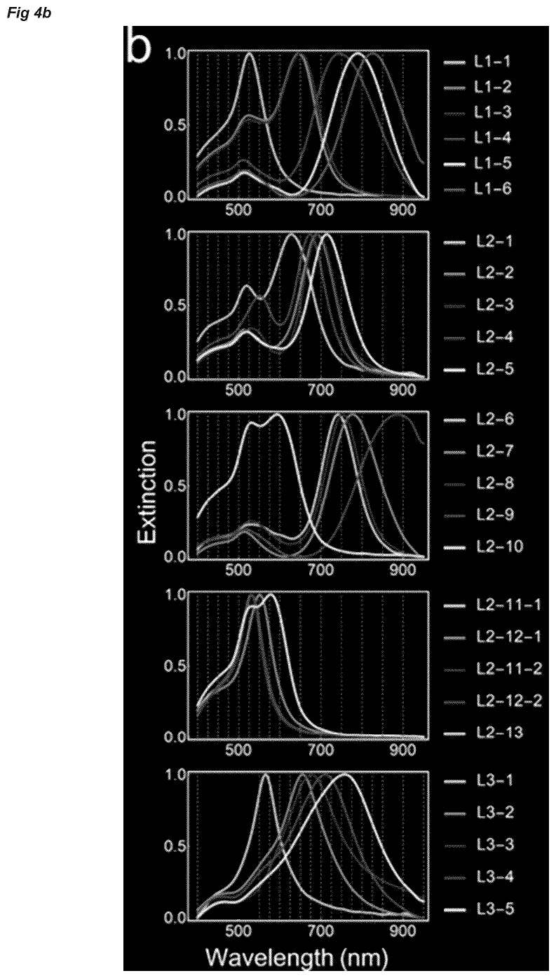

Three geometric parameters including the width, length and aspect ratio of the nanorods (L1-2 to L1-6) were measured, with the corresponding PDI summarized in Table 14. The histograms of the distributions of these three parameters corresponding to different samples (L1-2 to L1-6) is not shown here. L1-2 and L1-3 showed very similar distributions for every geometric parameter, which is consistent with their similar UV-Vis spectra as shown in FIG. 4b. The increased aspect ratio from L1-2/L1-3 to L1-6 is also consistent with the red-shifted longitudinal peak as observed in the corresponding UV-Vis spectra.

TABLE 14

| | Width | PDI | Length | PDI | | PDI |
|---|---|---|---|---|---|---|
| Sample | (nm) | (Width) | (nm) | (Length) | Aspect ratio | (Aspect ratio) |
| L1-2 | 12.68 ± 1.20 | 0.009 | 28.37 ± 2.72 | 0.009 | 2.26 ± 0.32 | 0.020 |
| L1-3 | 13.16 ± 1.60 | 0.015 | 27.99 ± 3.34 | 0.014 | 2.15 ± 0.33 | 0.024 |
| L1-4 | 11.67 ± 1.67 | 0.020 | 35.45 ± 3.25 | 0.008 | 3.08 ± 0.37 | 0.014 |
| L1-5 | 11.18 ± 1.69 | 0.023 | 39.13 ± 4.43 | 0.013 | 3.57 ± 0.61 | 0.029 |
| L1-6 | 7.59 ± 0.73 | 0.009 | 30.45 ± 3.34 | 0.012 | 4.03 ± 0.48 | 0.014 |

The geometric parameters and their corresponding PDI of nanorod samples L1-2, L1-3, L1-4, L1-5 and L1-6. The width, length and aspect ratio are shown in the format of mean ± standard deviation. The corresponding PDI for every parameter was calculated via Eq.(33).

### Chemical Space 2

### Nanospheres (L2-11-2 and L2-12-2):

The average sizes for both nanosphere samples (L1-11-2 and L1-12-2) were estimated. The means and standard deviations of the diameters and the corresponding PDI are summarized in Table 15. The histograms of the distributions of the diameters of L1-11-2 and L1-12-2 are not shown here.

TABLE 15
The measured diameters and the
corresponding PDI of L2-11-2
to L2-12-2. The diameters are shown
in the format of mean ±
standard deviation.

| | Diameter | PDI |
|---|---|---|
| Sample | (nm) | (Diameter) |
| L2-11-2 | 42.48 ± 2.31 | 0.003 |
| L2-12-2 | 45.02 ± 2.18 | 0.002 |

[symbol]

[symbol]

indicates data missing or illegible when filed

### Nanorods (L2-1 to L2-10, and L2-13):

Three geometric parameters including the width, length, and aspect ratio of the nanorods (L2-1 to L2-10, as well as L2-13) were measured and summarized in Table 16. The histograms of the distributions of these three parameters corresponding to different samples (L2-1 to L2-10, together with L2-13) are not shown here. Au nanorods with spherical caps showed a red-shifted longitudinal peak with the increased aspect ratio (L2-13, L2-10, L2-1, L2-2). A similar phenomenon was observed for Au nanorods with rectangular caps (L2-3, L2-5, L2-7 and L2-9). Although L2-1 and L2-3 have similar aspect ratios, the longitudinal peak of Au nanorods with rectangular caps (L2-3) is more red-shifted compared to the ones with spherical caps (L2-1). This is consistent with the previous studies about the effects of the end-cap shape of Au nanorods on the extinction spectrum (57).

TABLE 16

| | Width | PDI | Length | PDI | | PDI |
|---|---|---|---|---|---|---|
| Sample | (nm) | (Width) | (nm) | (Length) | Aspect ratio | (Aspect ratio) |
| L2-1 | 19.04 ± 1.72 | 0.008 | 45.86 ± 5.18 | 0.013 | 2.43 ± 0.36 | 0.022 |
| L2-2 | 18.08 ± 1.27 | 0.005 | 51.00 ± 4.45 | 0.008 | 2.84 ± 0.36 | 0.016 |
| L2-3 | 29.73 ± 1.72 | 0.003 | 70.26 ± 6.37 | 0.008 | 2.37 ± 0.25 | 0.011 |
| L2-4 | 28.21 ± 3.03 | 0.012 | 54.69 ± 4.92 | 0.008 | 1.95 ± 0.22 | 0.013 |
| L2-5 | 19.98 ± 1.58 | 0.006 | 61.57 ± 5.67 | 0.008 | 3.11 ± 0.41 | 0.017 |
| L2-6 | 15.66 ± 1.81 | 0.013 | 46.63 ± 4.48 | 0.009 | 3.01 ± 0.42 | 0.019 |
| L2-7 | 22.33 ± 2.09 | 0.009 | 75.13 ± 6.92 | 0.008 | 3.40 ± 0.48 | 0.020 |
| L2-8 | 16.52 ± 1.48 | 0.008 | 48.52 ± 5.20 | 0.011 | 2.95 ± 0.37 | 0.016 |
| L2-9 | 16.65 ± 1.18 | 0.005 | 75.64 ± 7.40 | 0.010 | 4.57 ± 0.61 | 0.018 |
| L2-10 | 26.36 ± 1.53 | 0.003 | 51.95 ± 4.60 | 0.008 | 1.98 ± 0.21 | 0.011 |
| L2-13 | 28.27 ± 1.71 | 0.004 | 48.67 ± 4.94 | 0.010 | 1.73 ± 0.20 | 0.013 |

The geometric parameters and their corresponding PDI of nanorod samples L2-1 to L2-10, together with L2-13. The width, length and aspect ratio are shown in the format of mean ± standard deviation. The corresponding PDI for every parameter was calculated via Eq.(33).

### Time Cost Analysis During Exploration

To give a detailed insight into the time cost during the autonomous exploration, we recorded the time cost of individual operations (including solution preparation, waiting for a complete growth and UV-Vis characterisation and cleaning) during the exploration of chemical space 2 and 3 (30 steps, 720 experiments in total). In both cases, the waiting time for growth of a batch of samples was set as 60 minutes. The UV-Vis analysis and cleaning took 98 minutes through the whole 30 steps, regardless of the required synthetic conditions.

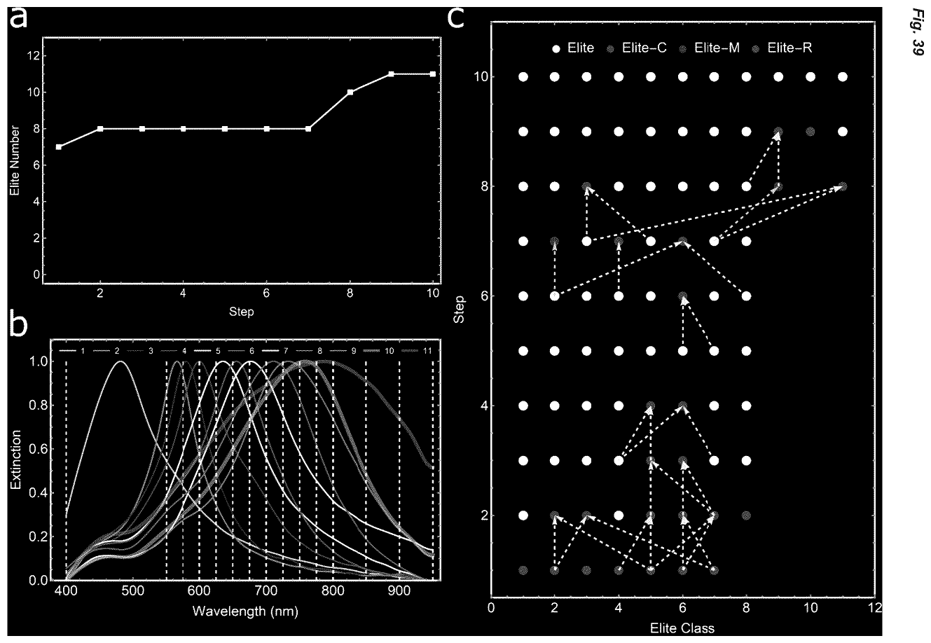

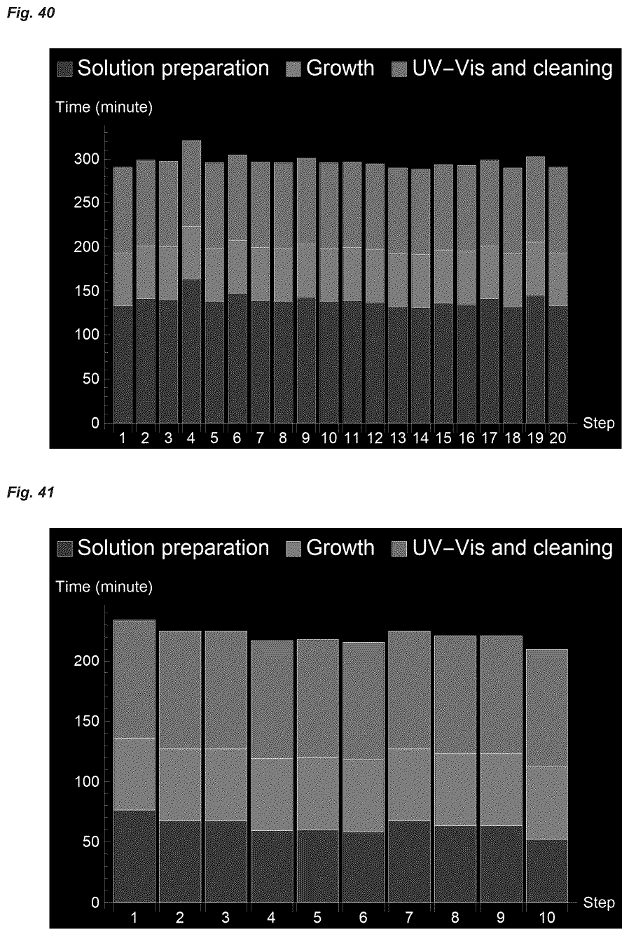

The time cost is higher in exploring the chemical space 2 due to the waiting time for a stable pH read-out and the process to control the pH. The time cost for liquid dispensing/pH control varied in every step, depending on the synthetic conditions generated for that step (139±7 minutes, see FIG. 40). Thus, the total time cost for a single step including 24 experiments took 297±7 minutes (ca. five hours). During the exploration of chemical space 3, only liquid dispensing was required, and the preparation time was decreased to 63±7 minutes (see FIG. 41), thus giving a total 221+7 minutes (ca. three and a half hours) for one complete step.

### 4. Optimisation Towards Specific Optical Properties

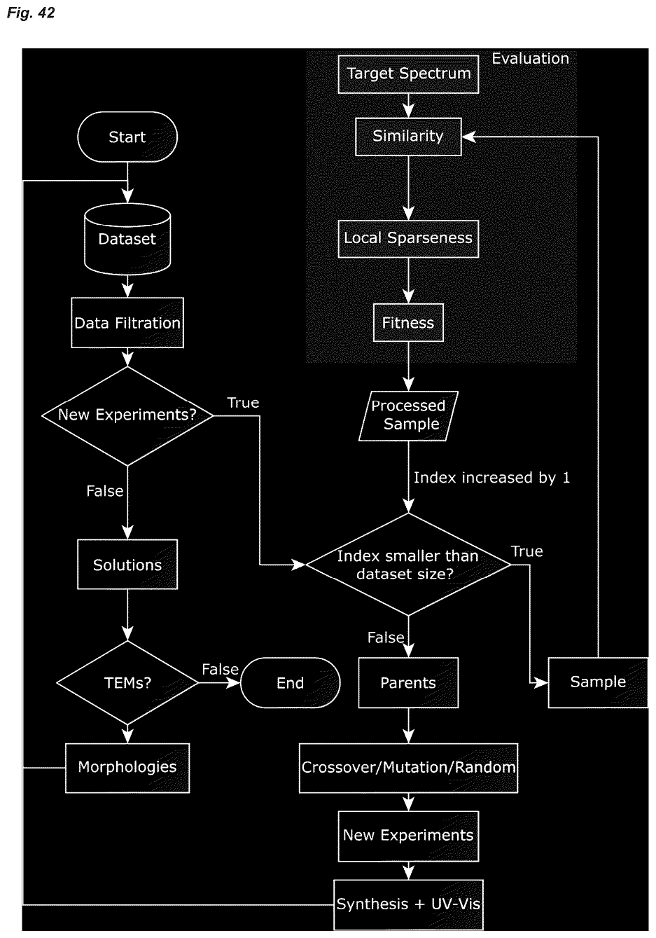

The experimental implementation of the optimisation strategy based on GS-LS will be introduced in this section. The flow diagram of the strategy is shown in FIG. 42. The procedure for the optimisation was demonstrated as following:
1. After exploring the chemical space, a simulated target spectrum can be reasonably generated from the available nanostructures via discrete-dipole-approximation simulation. The aim is to find multiple solutions with similar UV-Vis features as this target, but with distinct synthetic conditions.
2. All data available from previous exploration of this given chemical space is collected.
3. Data filtration:
   a. The UV-Vis is tested to make sure it is not too noisy (same as described in the exploration).
   b. Confirm that there is at least one peak present.
   c. The prominence of at least one peak is larger than a threshold (0.2).

In this work, we are concerned with nanostructures large enough to exhibit UV-Vis peaks therefore both the target and relevant experimental spectra should contain peaks. This is the reason for criteria (b and c) above. Sampling points with spectra failing to meet either/both of these criteria are discarded.
4. For every sample in the dataset that passing the filtration in step 3:
   a. The sample's UV-Vis will be compared with the target spectrum to measure their similarity.
   b. The local sparseness near the sample is calculated using the filtrated data.
   c. The fitness function is defined by the linear summation of the similarity and local sparseness.
5. The samples with the top N highest fitness are selected as the parents and new experiments will be designed by the crossover and mutation within the parents, together with a small portion of random sampling.
6. The autonomous platform conducts the new experiments and updates the dataset.

Steps 3-6 above were iterated until the optimisation was over. The local sparseness of the sample was updated during the iteration. The final solutions, with the highest similarity metric among its K-nearest neighbours, will be selected from the dataset. They are further reproduced and characterised with TEMs to check the resulting morphologies.

The optimisation strategy was demonstrated with two cases. In this first case, the UV-Vis from Au nanorod of a specific size was set as the target spectrum. After optimisation two samples with almost identical UV-Vis spectra but separated in the input chemical space were found to match the target. Both of them correspond to Au nanorods. In the second case, the target spectrum is simulated from Au octahedra. Multiple solutions with different morphologies including normal octahedra, concave octahedra, smooth polyhedra and mixtures were found to match the target spectrum.

### 4.1 Similarity, Local Sparseness, and Fitness

The optimisation algorithm was implemented based on GS-LS using evolutionary algorithm (EA) as the optimiser. After setting the target spectrum, the similarity (M_{S}) and local sparseness(S) metrics were defined as discussed before (Eq. (1)-Eq. (3)).

$$S ( x ) = \frac{1}{K} {\sum }_{i = 1}^{K} dist ( x , {x}_{i}^{′} ) \qquad ( 1 )$$

where dist(x, x′ᵢ) measures the Euclidean distance between x and x′ᵢ, and x′ᵢ is the i^{th} closest sample to x. In both optimisation cases, we evaluated the ten-nearest neighbours for local sparseness. Note that multiple points may have the same input condition, so the duplicated input points were deleted before calculating the local spareness.

$${M}_{S} = - \vert p - {p}_{t a r g e t} \vert - {k}_{1} {\sum }_{i} \vert {I}_{x , i} - {I}_{t a r g e t , i} \vert + {k}_{2} \qquad ( 2 )$$

where p and p_{target} are the peak positions of the highest peak in the UV-Vis spectra of the sample and the target. I_{x,i} and I_{target,i} are the i^{th} intensity of the UV-Vis data of the sample and the target respectively. We set k₁=0.2 and k₂=0 in the optimisation. It should be noted the unit of |p−p_{target}| here is set as nanometer. This setting of fitness function tends to find samples with the same peak position while maintaining spectral similarity.

$$F = {M}_{S} + {k}_{3} S \qquad ( 3 )$$

where S is the local sparseness term and k₃ was set as 100.

The top five samples with the highest fitness (F) defined by Eq. (3) were selected as the parents for crossover and mutation to generate the new experiments. The ten nearest unique neighbours (including itself) are used to calculate the local sparseness. The local sparseness term was updated from step to step. T here are 23 samples generated per step and among these 23 samples, 10 samples were mutated from the parent set, 10 samples were from crossover among parents with a further 40% chance of mutation and 3 samples were randomly generated in the input chemical space. The multi-Gaussian distribution in the mutation was set with a mean of 0 and a standard deviation of 0.08 for all dimensions. The boundary condition was maintained in a similar way as discussed above (FIG. 29). The procedure to process the UV-Vis signal including smoothing, normalizing, and discarding spectra is the same as described in exploring chemical space 1 (Section 3.2). Note all the UV-Vis spectra were normalized before data processing.

After the optimisation, the solutions were selected by comparing the similarity metric with its six nearest neighbours (including itself). Only if the sample's similarity metric is no less than these neighbours', the sample is regarded as a local maximum in the observation set and returned as a solution.

### 4.2 Target UV-Vis from Rods

After the exploration of chemical space 1, a target spectrum was set considering the existence of nanorods. The target spectrum was from the DDA simulation of cylindrical Au nanorods with a diameter of 11 nm and a length of 33 nm. The experimental details and boundary conditions in optimisation towards Au nanorods are the same as those in the chemical space 1 (Section 3.2). The linear transformation between input variables and reagent volumes is the same as that in Section 3.2. The initial data set is from the exploration of chemical space 1 (16 steps in total including the first step of random sampling). The optimisation was run for 5 steps with the initial dataset.

### Stability of the System

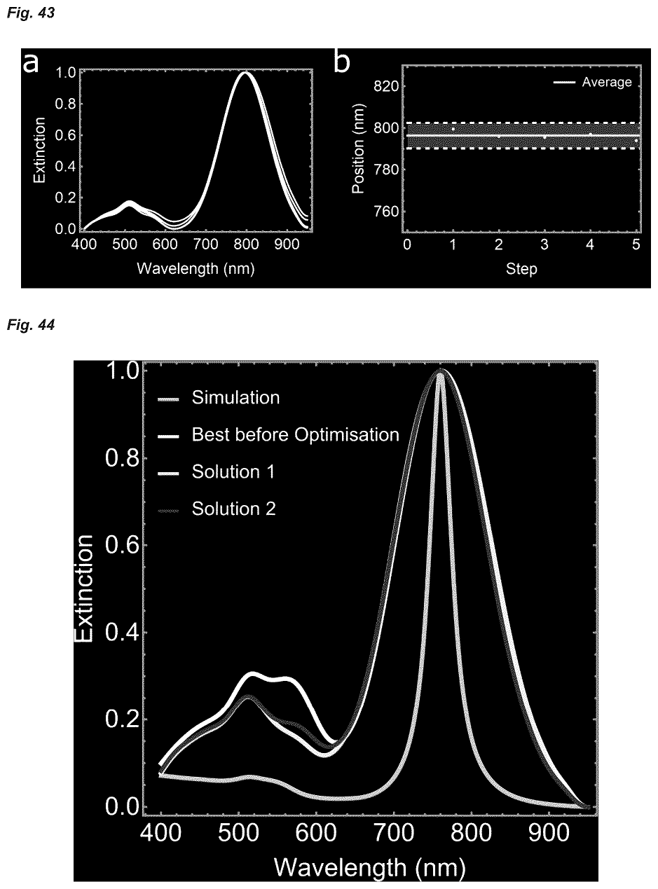

During the optimisation, standard samples with unchanged condition were synthesised (Table 4-1 and FIG. 43) to track the stability of the platform.

TABLE 4-1

| | CTAB | HAuCl₄ | AgNO₃ | Ascorbic | Water | Seed |
|---|---|---|---|---|---|---|
| Standard | (mL) | (mL) | (mL) | acid (mL) | (mL) | (mL) |
| 4 | 4.40 | 2.50 | 1.80 | 1.10 | 1.80 | 0.50 |

The input parameters as the standard to test the stability of the autonomous platform in the optimisation.

TABLE 4-2

| | CTAB | HAuCl₄ | AgNO₃ | Ascorbic | Water | Seed |
|---|---|---|---|---|---|---|
| Solution | (mL) | (mL) | (mL) | acid (mL) | (mL) | (mL) |
| B | 4.63 | 2.59 | 2.88 | 1.40 | 0.00 | 0.50 |
| 1 | 5.41 | 2.79 | 1.12 | 1.51 | 0.67 | 0.50 |
| 2 | 4.63 | 2.08 | 1.39 | 1.40 | 2.00 | 0.50 |

The synthetic conditions of solutions for the rod target. The reagents used here have the same concentrations as those in chemical space 1. B indicates the best solution before the optimisation.

After the optimisation, we selected the top two solutions (Table 4-2) with highest similarity for TEM characterisation. Before the optimisation, polyhedral by-products were clearly present in this sample of the best solution as indicated in FIG. 44 (shape yield of rods is ca. 57% by counting ca. 900 nanoparticles). The peak around 570 nm was due to these by-products in the sample. After the optimisation, the multiple solutions showed a decreased by-product peak with similar desired UV-Vis features. TEM characterisation indicates the UV-Vis features were due to Au nanorods. The solution 1 shows the higher yield of target nanorods (shape yield of rods is ca. 95% by counting ca. 1100 nanoparticles), which is consistent with the small by-product peak as observed in the UV-Vis. Solution 2, despite exhibiting close similarity to the target and a marginally higher fitness than solution 1, was revealed to have the lesser nanorods of the two solutions. This reveals the strength of this method, in that it will provide multiple solutions to a problem, increasing the robustness of producing a truly optimised sample.

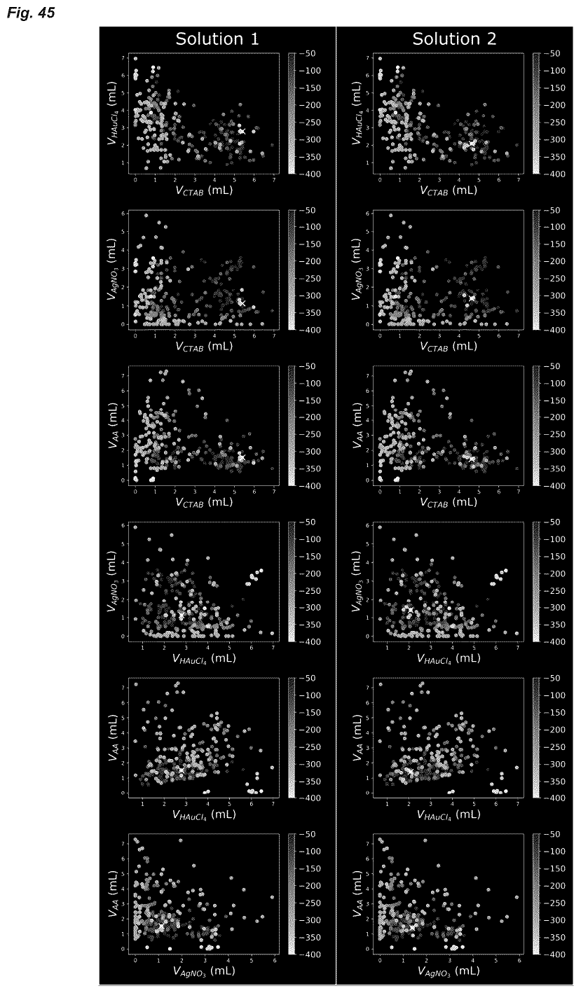

These two solutions were selected by the criteria of K-nearest neighbours so that they have the highest similarity metric comparing to its six nearest neighbours (including itself). In calculating the distance between sampling points, the normalized variables in the range from 0 to 1 instead of the reagent volumes were used. The synthetic conditions of both solutions together with their nearest neighbours were labelled as cross and red points respectively in FIG. 45. The similarity metric distribution of the rest samples with various synthetic conditions are shown as well.

### 4.3 Target UV-Vis from Octahedra

After exploration of chemical space 3, the emergence of octahedral features was observed in Au nanostars (L3-1,), and octahedral nanoparticles were observed in another sample (Elite 3). Thus, a target spectrum from the DDA simulation of Au octahedra with a longer axis length of ca. 80 nm (edge length of 80/\sqrt{2}≈56.5 nm) was set. The optimisation was expected to amplify the octahedral feature in this chemical space. The experimental details and boundary conditions in the optimisation towards octahedra are the same as those in the chemical space 3 (Section 3.4) except for the concentration of HAuCl₄ which is halved to 0.5 mM. This is because after exploring chemical space 3, all the available data were compared with the target spectrum. The volumes of HAuCl₄ used in the best five samples are all below 1.00 mL, as a result the chemical space was shrunk in the optimisation.

The linear transformation between input variables in the algorithm and volumes of the reagents is the same as described in Section 3.4. The only difference was the concentration of HAuCl₄, which means the maximum concentration of HAuCl₄ in the growth solution was halved compared to that during exploring chemical space 3. Thus, the chemical space where the optimisation will happen was shrunk. As a result only data from the original chemical space 3 dataset, within the bounds of the shrunk chemical space were used. The optimisation was run for 5 steps starting with the initial dataset.

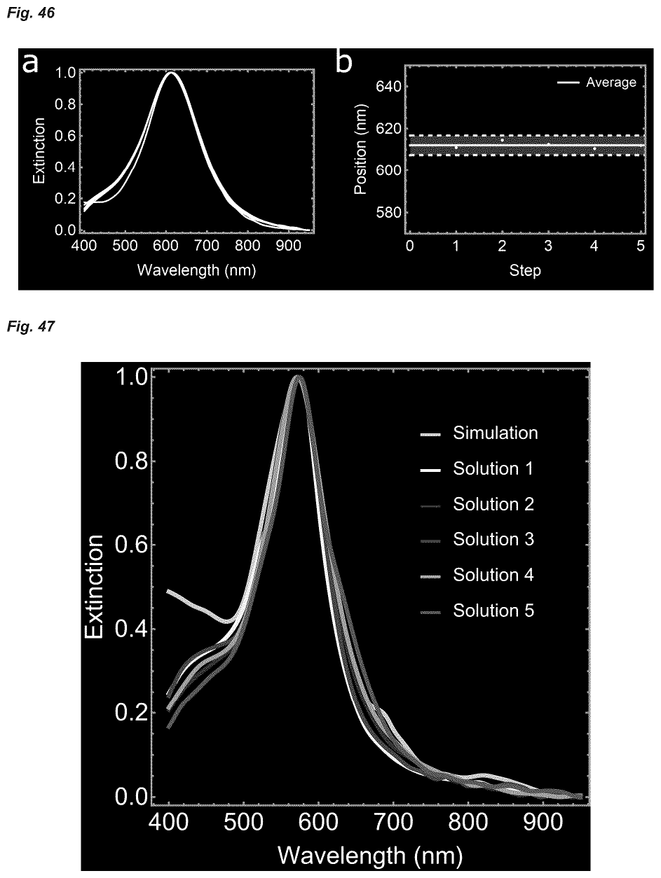

Again, standard samples with unchanged synthetic condition in the same shrunk chemical space were used to track the stability of the autonomous platform (Table 4-3 and FIG. 46).

TABLE 4-3

| | CTAC | HAuCl₄ | AgNO₃ | HCl | Ascorbic | Water | Seed |
|---|---|---|---|---|---|---|---|
| Standard | (mL) | (mL) | (mL) | (mL) | acid (mL) | (mL) | (mL) |
| 5 | 1.57 | 1.43 | 1.37 | 0.60 | 5.00 | 1.53 | 0.50 |

The input parameters as the standard to test the stability of the autonomous platform in the optimisation.

TABLE 4-4

| | CTAC | HAuCl₄ | AgNO₃ | HCl | Ascorbic | Water | Seed |
|---|---|---|---|---|---|---|---|
| Solution | (mL) | (mL) | (mL) | (mL) | acid (mL) | (mL) | (mL) |
| 1 | 0.75 | 0.50 | 4.15 | 2.25 | 1.24 | 2.61 | 0.50 |
| 2 | 2.43 | 1.26 | 0.19 | 0.31 | 5.51 | 1.80 | 0.50 |
| 3 | 0.12 | 0.63 | 2.08 | 5.08 | 0.59 | 3.00 | 0.50 |
| 4 | 1.22 | 1.26 | 0.19 | 0.31 | 5.51 | 3.01 | 0.50 |
| 5 | 4.24 | 0.91 | 0.76 | 2.57 | 2.89 | 0.13 | 0.50 |

The synthetic conditions of the solutions for octahedral target. The reagents used here have the same concentrations as those in chemical space 3 except that the concentration of HAuCl₄ was decreased to half (0.5 mM).

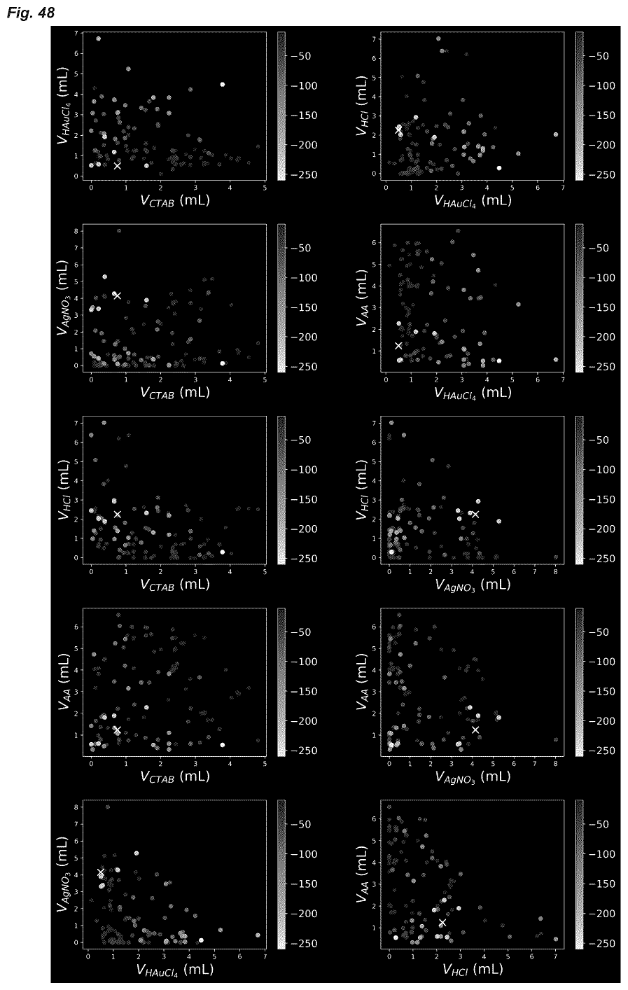

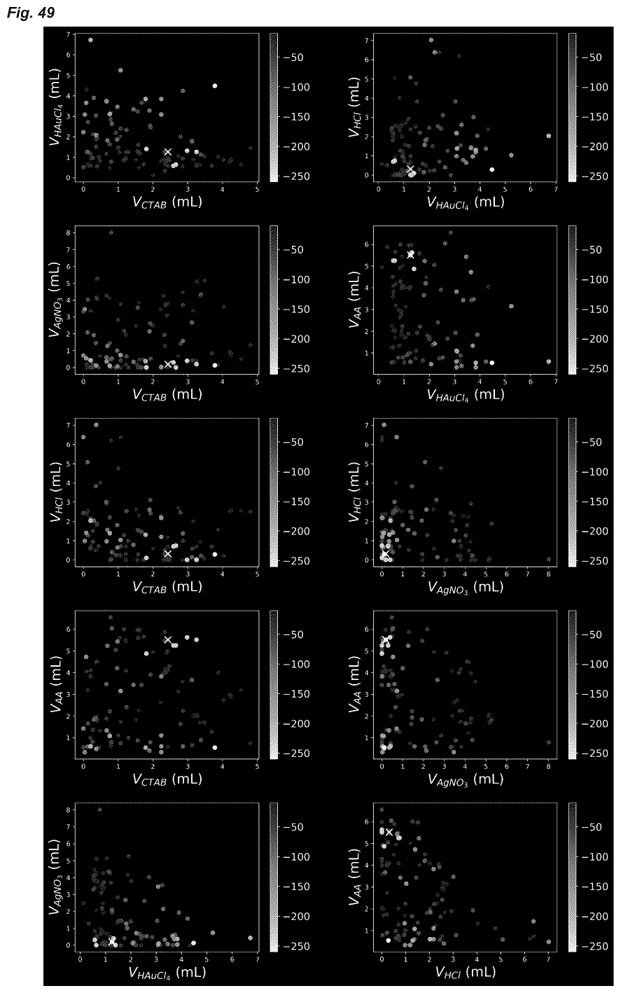

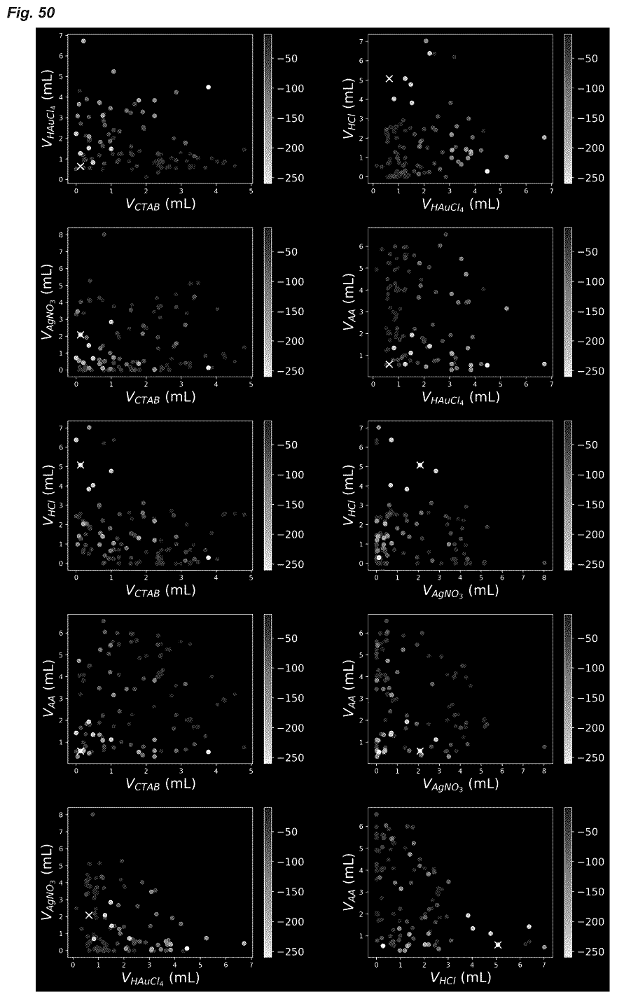

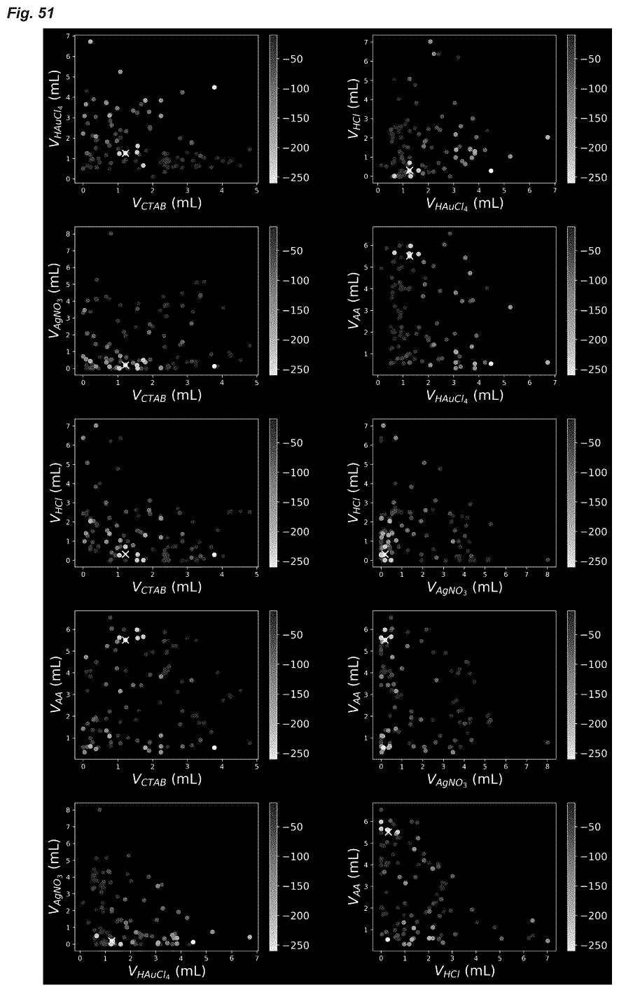

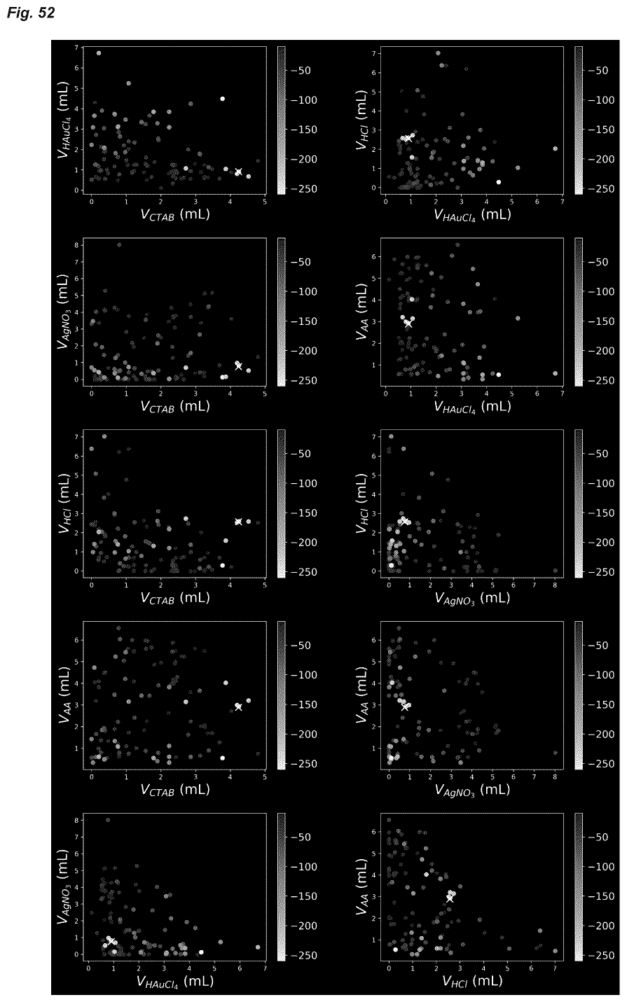

After the optimisation, all the data (from both exploration and optimisation) in the shrunk chemical space were used further to give the final solutions. Again, the K-nearest neighbour criteria was implemented to find local maxima regarding the observation set. Each solution was selected so that its similarity metric was no less than its six nearest neighbours (including itself). The synthetic conditions of the top five solutions with the highest similarity and their corresponding UV-Vis spectra are shown in Table 4-4 and FIG. 47. The distribution of the five solutions in the chemical space and their corresponding neighbours are shown in FIGS. 48-FIG. 52.

### 5. Autonomous Synthesis of Nanoparticles Via Multistep Growth

In this section, the fully autonomous synthesis of multiple batches of Au NPs is introduced, where many of the NPs are both products and required as seeds for nanoparticles of higher complexity. Their unique digital signatures would be generated after the synthesis.

Considering the synthesis of nanoparticles, three steps are necessary:
1. Define the desired nanoparticles and their hierarchical relationship as a synthetic network.
2. Design many identical reactions that are needed to satisfy the multiple requirements of a single nanoparticle. Several samples would be needed for analysis, for seeding material for the next reaction level and for potential applications. Repeated parallel reactions to generate enough volume of one nanoparticle must therefore exist.
3. Define the necessary operations that are required to conduct the chemical reactions on the autonomous platform.

Directed graphs can handle complicated networks and are easy to visualize. Thus, they are used to satisfy the three steps above.

### 5.1 the Directed Synthesis Graph, Reaction Graph and Hardware Graph

The three directed graphs were defined as following:
1. The directed synthesis graph. A graph is created according to the synthetic routines for multiple nanoparticles. The nanoparticles are represented by nodes. The hierarchical relationship between nanoparticles, which is defined by using one nanoparticle as the seed for another, is indicated by the directed edges among the nodes.
2. The directed reaction graph. A graph is automatically generated using the synthesis graph to determine the experimental design. In designing the experiments, the multiple repeated synthesis of the same nanoparticle for its UV-Vis characterisation or seeding of new reactions are considered. The node in the reaction graph represents one sample of a nanoparticle, while the directed edges indicate which samples will be used as the seeds for new reactions.
3. The directed hardware graph. A graph is generated from the reaction graph and used to define the operations that will be conducted in the platform. The available experimental resources from the hardware are distributed among the parallel synthesis of multiple samples. In our system, this graph represents the distribution of samples in the available vials on the wheel, defines the seed transfer operation among the samples, and assigns samples for UV-Vis characterisation. All the operations to be executed on the platform are defined by the hardware graph.

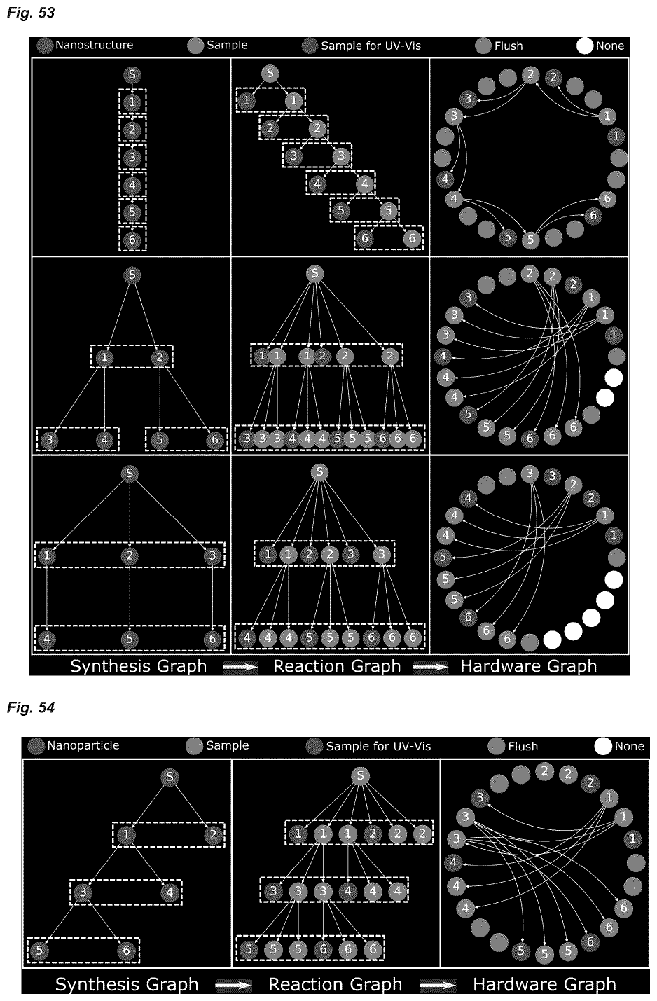

With the directed graph structure, the platform can handle various synthetic networks containing multiple nanoparticles performed in parallel. The synthesis graphs of different synthetic networks are shown in FIG. 53, left column. The chemical reactions to achieve multiple nanoparticles in different networks were designed according to the synthesis graph and recorded in the reaction graph (FIG. 53, middle column). Finally, the hardware graph was generated according to the reaction graph. The samples to be synthesised were mapped to the available slots on the wheel. The seed transfer operation from one sample to others were then defined (FIG. 53, right column).

The number and durations of the growth steps to reach different nanoparticles can vary. Depending on the number of steps, the nanoparticles are divided into different batches, which are indicated by the different layers in the synthesis graph or reaction graph (FIG. 53, left and middle columns). The synthesis was conducted batch by batch. Between batches, UV-Vis was used to validate the sample reproducibility before using it as the seeds. Since multistep synthesis may require long growth time from batch to batch, pre- and post-reaction wash/flush routines were performed after each batch of reactions to ensure no contamination in transfer. Note that depending on the synthesis graph, not all of the vial slots are used in the complete process. See the hardware graphs in FIG. 53 middle and bottom rows for examples.

The graph representation of the synthesis of many nanoparticles with clearly defined chemical reactions and the set-up of the hardware offers a generic way to both present and set up the multistep synthesis in our system. The source code to generate the graphs is available at https://github.com/croningp/NanomatDiscovery.

### 5.2 The Autonomous Synthesis of Desired Nanoparticles

To validate the directed graph strategy described above and the reproducibility of the autonomous platform, we synthesised six nanoparticles of varying shapes including rods, sphere and stars, which were all discovered in the exploration. They are labelled from N1 to N6 (which correspond to L1-5, L1-1, L2-12-12-2, L2-7, L3-3 and L3-1 as discussed before). N1 and N2 are from chemical space 1 which correspond to small Au nanorods and nanospheres; N3 and N4 are from chemical space 2 that correspond to large Au nanorods and nanospheres; and N5 and N6 are from chemical space 3 that correspond to Au nanostars. Up to three steps of growth was required to complete this series which can be seen in FIG. 54.

### 5.2.1 Experimental Details

### Chemicals and Stock Solutions:

Type I ultrapure water. (18.4 MΩ·cm)
Hexadecyltrimethylammonium bromide, >99%, Acros Organics, CTAB. (0.2M)
Hexadecyltrimethylammonium chloride, >99%, Acros Organics, CTAC. (0.2M)
Gold (III) chloride trihydrate, 99.9%, Sigma-Aldrich, HAuCl₄.
(1.0 mM for overgrowth solution and 0.5 mM for seed preparation)
Ascorbic acid, 99.9%, Sigma-Aldrich (13.1 mM)
Silver nitrate, 99.9999%, Sigma-Aldrich, AgNO₃ (0.25 mM)
Hydroquinone, 99.5%, Acros Organics. (13.1 mM)
Hydrochloric acid, ACS Reagent, 37%, Honeywell Fluka™, diluted with Type I water. (0.1 M)
Sodium hydroxide, 98%-100.5%, Honeywell Fluka™, NaOH. (0.1 M)

The initial 2 nm Au seed was synthesised as described in Section 3.2. Note ascorbic acid (13.1 mM) was used in the multistep synthesis and the volume of the reagents were changed accordingly to maintain their concentrations to the original synthetic conditions. Three batches of N1-N2, N3-N4 and N5-N6 were aged for 2, 16 and 1 hours, respectively. Every nanoparticle was repeated 3 times, and one of the repeats was used for the UV-Vis characterisation. Here, the water reference of the UV-Vis was taken before pumping in the samples, where the flow cells were cleaned and filled with Type I water. The synthetic conditions of these six nanoparticles are listed in Table 5-1.

TABLE 5-1
Batch 1 (growth for 2 hours)

| | CTAB | HAuCl₄ | AgNO₃ | Ascorbic | Water | Seed |
|---|---|---|---|---|---|---|
| Label | (mL) | (mL) | (mL) | acid (mL) | (mL) | (mL) |
| N1 (L1-5) | 4.66 | 2.83 | 1.27 | 0.29 | 2.45 | 0.50 |
| N2 (L1-1) | 1.51 | 2.53 | 0.09 | 1.78 | 5.59 | 0.50 |

Batch 2 (growth for 16 hours)

| | CTAB | Hydroquinone | Water | | AgNO₃ | HAuCl₄ | Seed |
|---|---|---|---|---|---|---|---|
| Label | (mL) | (mL) | (mL) | pH | (mL) | (mL) | (mL) |
| N3 (L2-12-2-2) | 1.85 | 1.07 | 4.08 | 6.68 | 0.07 | 3.09 | 0.50 |
| N4 (L2-7) | 4.17 | 2.54 | 0.29 | 4.22 | 2.30 | 2.70 | 0.50 |

Batch 3 (growth for 1 hour)

| | CTAC | HAuCl₄ | AgNO₃ | | Ascorbic | Water | Seed |
|---|---|---|---|---|---|---|---|
| Label | (mL) | (mL) | (mL) | HCl (mL) | acid (mL) | (mL) | (mL) |
| N5 (L3-3) | 6.81 | 1.92 | 0.03 | 1.19 | 0.60 | 0.95 | 0.50 |
| N6 (L3-1) | 2.66 | 0.32 | 0.00 | 0.74 | 5.25 | 2.53 | 0.50 |

The synthetic conditions of N1 to N6. Note N1 is the seed for N3 and N4, and N3 is the seed for N5 and N6. When reproducing N3 and N4, the operations of adding acid or base sequentially were conducted as recorded in preparing the original solutions without measuring the pH. Note ascorbic acid (13.1 mM) was used.

### 5.2.2 The Graph Representation of N1 to N6

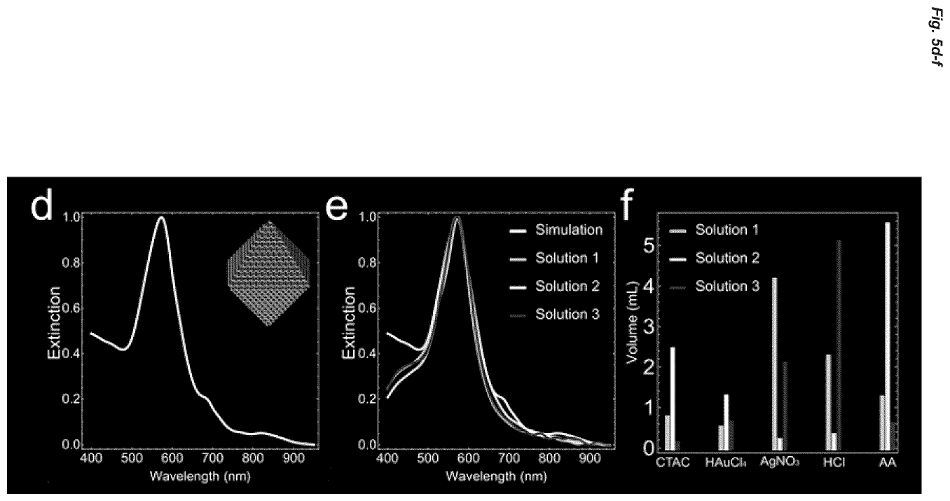

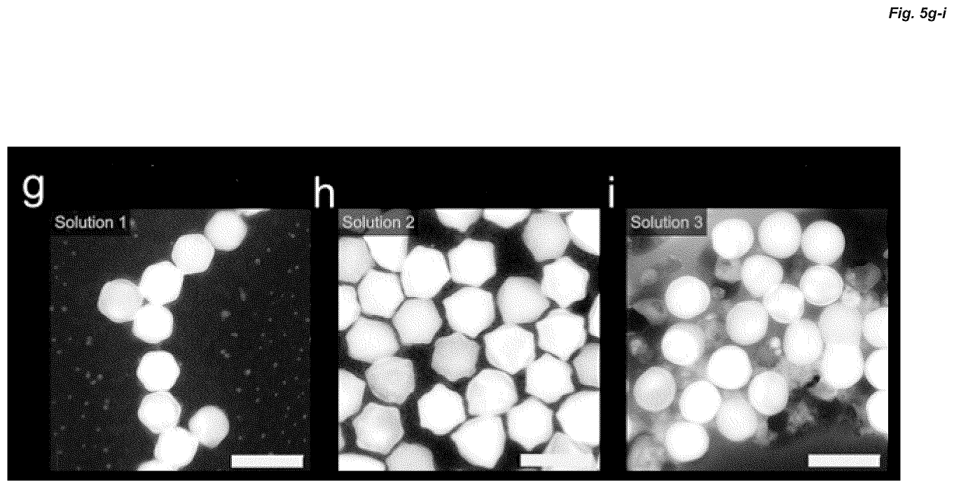

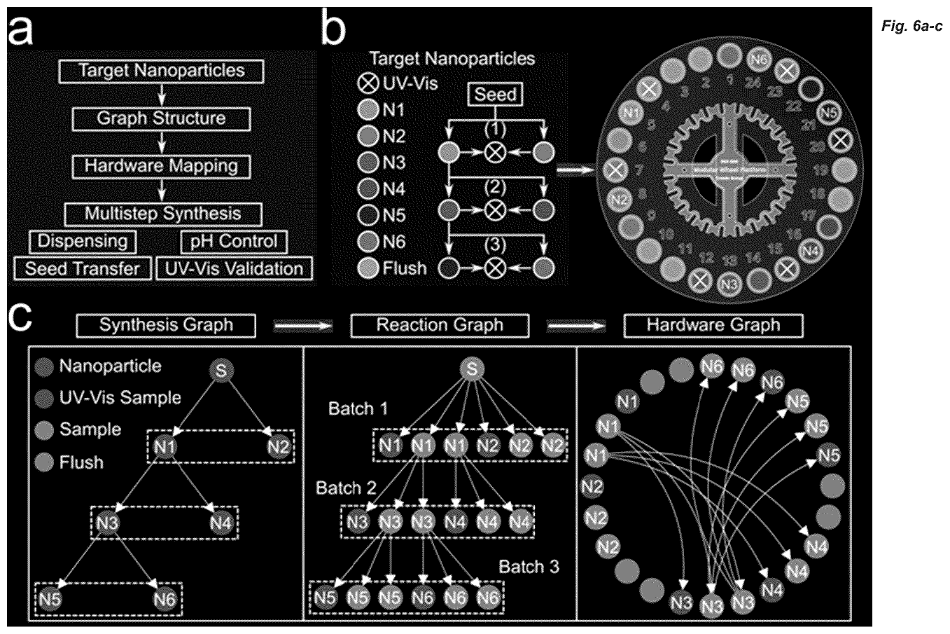

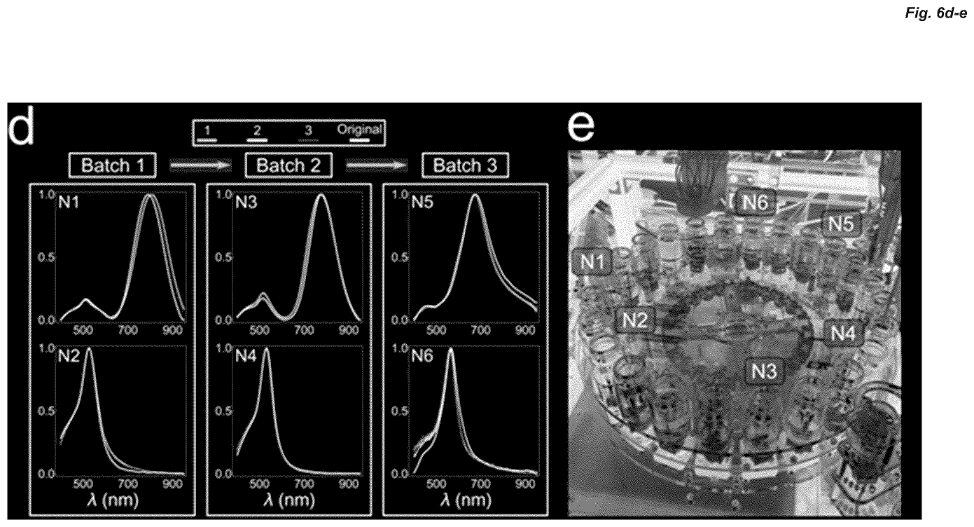

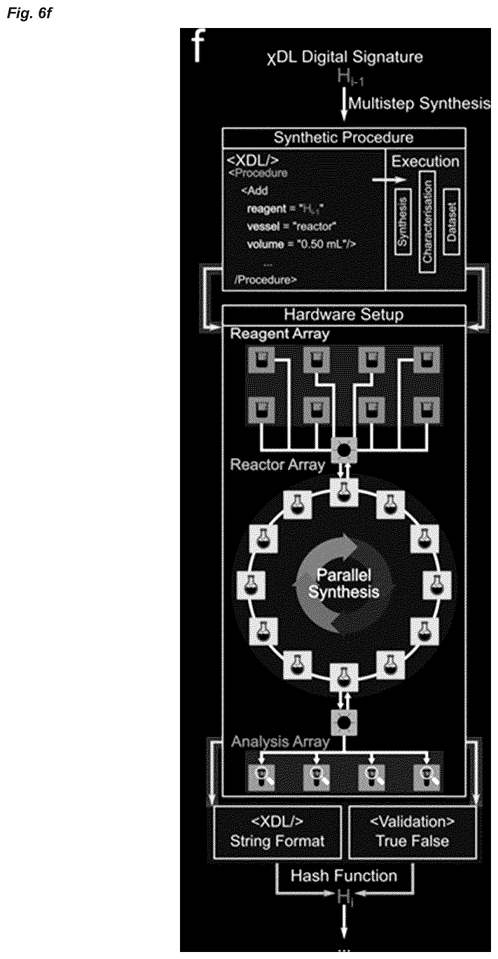

The corresponding synthesis graph, reaction graph and hardware graph are shown in FIG. 54. The autonomous multistep synthesis was repeated three times independently to validate the stability of the system. The average highest peak position (including three repeats and the original spectrum from exploration) with their standard deviation are shown in Table 5-2. These spectra were only normalized to the range from 0 to 1 without being processed by the low-pass filter to reflect the possible detection noise. The comparison of UV-Vis spectra between the target and the three repeats are shown in FIG. 6, which demonstrates the high synthetic reproducibility offered by the autonomous platform.

TABLE 5-2
The average peak position and also the
standard deviation of the highest peak for
N1 to N6.

| | | Standard |
|---|---|---|
| | Average Peak | Deviation |
| Label | Position (nm) | (nm) |
| N1 (L1-5) | 799.6 | 6.5 |
| N2 (L1-1) | 525.9 | 0.9 |
| N3 (L2-12-2-2) | 530.0 | 1.2 |
| N4 (L2-7) | 777.0 | 6.9 |
| N5 (L3-3) | 673.4 | 2.6 |
| N6 (L3-1) | 561.0 | 3.4 |

### 5.3 The Unique Digital Signature of Nanoparticles

It is important to create the unique digital signatures of nanoparticles from various synthetic conditions regarding their wide applications. Here we introduce how to create unique digital signatures using the information of synthetic procedure as well as the validation of synthesised nanoparticles. Since there are multiple ways to describe the same synthetic procedure, we implemented the universal chemical programming language, χDL¹¹, to describe the procedure. Depending on the type of nanoparticles, various techniques such as electron microscopy, dynamic light scattering, and small-angle X-ray scattering can be used to validate the synthesis of nanoparticles. Considering the plasmonic effect of Au NPs, in-line UV-Vis was used to validate the synthesis.

The digital signature of one nanoparticle sample can be generated using hash function as following:
1. Both the chemicals and the steps to obtain nanoparticles are described using χDL.
2. The chemical synthesis of the sample is conducted and the product is validated.
3. A “True” or “False” statement is appended to the string format of χDL depending if the sample is reproduced as desired.
4. The string including χDL as well as the True/False statement is encoded with UTF-8, and further converted to the unique digital signature using the hash function of SHA-256.

All the chemicals were represented using their CAS numbers, with corresponding concentrations in the unit of M (molar per litre). In the synthesis where a nanoparticle solution was used as the seed, its digital signature was used in the χDL description, and would participate in hashing the new nanoparticle.

### Discussion

The present invention relates to a system for the Autonomous Intelligent Exploration, DIScovery, and Optimisation of Nanomaterials (AI-EDISON), which aims for both discovery and reproducible multistep synthesis of novel nanomaterials, with their unique digital signatures derived from physical properties and synthetic procedures⁴⁶.

The experimental architecture performs parallel synthesis of nanomaterials together with real-time spectroscopic characterisation and is assisted by ML algorithms and an extinction spectrum simulation engine. AI-EDISON uses state-of-the-art quality-diversity algorithms to explore high-dimensional combinatorial synthetic space to perform open-ended exploration, and then conducts targeted optimisation to search optimal synthetic conditions for nanomaterials with finely tuned optical properties. It can be further used to perform multistep synthesis of any desired nanoparticles it has found with a resource efficient, directed graph strategy coupled with real-time characterization.

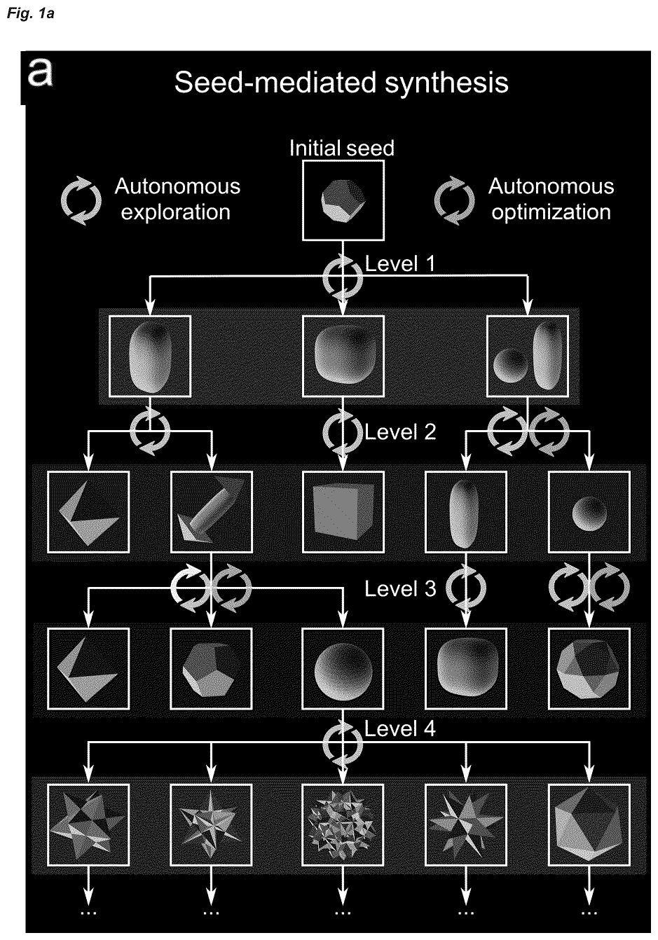

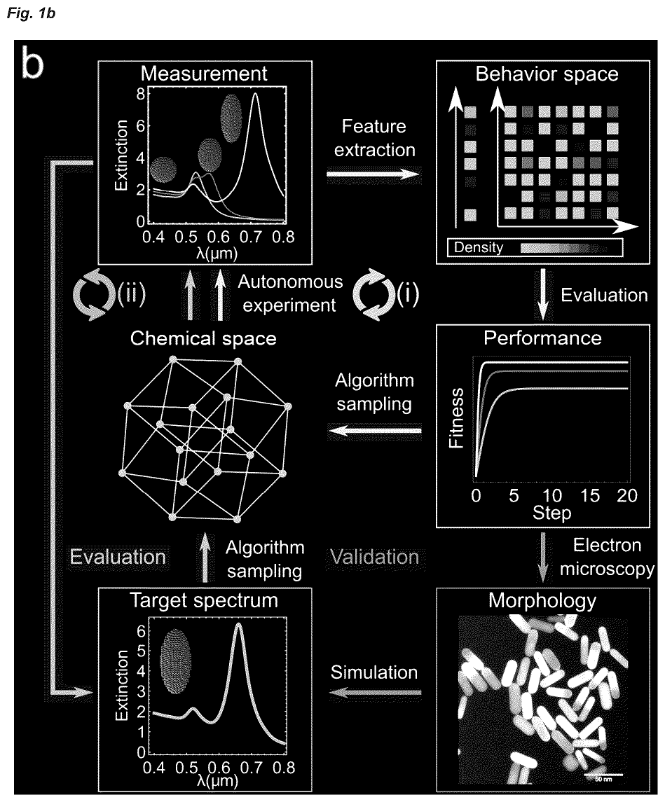

Using the directed graph approach, the complete multistep nanoparticle synthesis can be efficiently represented as a robust digital procedure, avoiding irreproducibility due to operation errors. With AI-EDISON, we investigated three chemical synthetic spaces connected by the seed-mediated synthesis of gold nanoparticles (AuNPs), where nanoparticles synthesised from the lower-level space were used as seeds in the higher-level space. By using UV-Vis spectroscopy as a primary characterisation technique, we started with the hypothesis that increasing the diversity in the spectra could lead to the efficient exploration of the chemical space with distinct nanostructures. After exploration, a simulation engine was used to create the targets to further optimise the optical properties of AuNPs. These linked chemical spaces initialised from a single physical seed with intermediate exploration and optimisation steps at various levels are represented in FIG. 1a.

The overall closed-loop algorithmic scheme used for the discovery of nanomaterials has two different modes: exploration and optimisation, see FIG. 1b. For each complete closed loop, AI-EDISON performs three different operations including nanoparticle synthesis, UV-Vis characterisation, and designing new experiments using ML algorithms. In the exploration mode, the structural diversity of the nanoparticles is achieved by searching for diversity in the behaviour space. This behaviour diversity is derived from the features observed in the UV-Vis spectra, such as peak number and position. The fitness, which is a numerical indication of the sample's performance, is evaluated based on peak prominence and broadness that correlate with yield and monodispersity of the nanoparticles. A new batch of experiments is generated from previous synthetic conditions leading to higher-performance samples and diversified features. The process including the three operations iterates until the exploration is completed. After the exploration, transmission electron microscopy (TEM) is used as a secondary characterisation technique to check nanoparticle morphologies with high-performance UV-Vis features. In the optimisation mode, a target spectrum is defined by the extinction spectrum simulation of the nanoparticle with the shape derived from electron micrographs. This strategy with the extinction spectrum simulation extends the optimisation targets to nanostructures with features that are not directly available in the exploration. Because of the lack of one-to-one mapping between UV-Vis spectra and nanostructures, various morphologies could lead to the similar spectra to the target. Hence, the algorithm considers similarity to the target spectrum and also the sampling density in the synthetic space to find multiple optimal conditions as solutions to the optimisation problem.

### AI-EDISON: Autonomous Nanomaterials Synthesis Robot and Characterisation

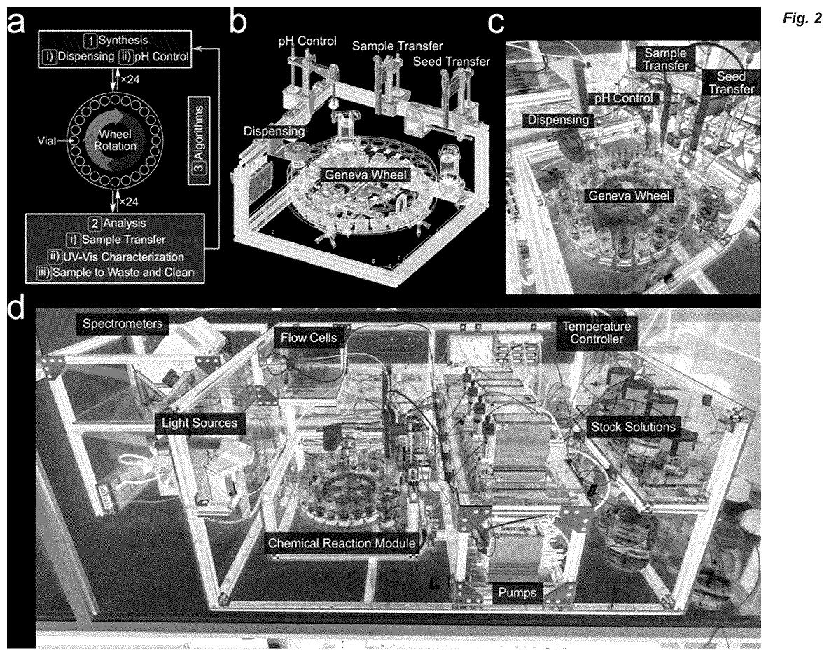

The core robotic hardware comprises a chemical reaction module capable of performing parallel synthesis up to 24 reactors²¹. The modular architecture utilises the rotation of the reactors which is synchronized with both parallel/sequential liquid dispensing and stirring of reagents to conduct the synthesis efficiently. Using a combination of precision syringe pumps, the control system performs liquid handling, mixing, cleaning, dynamic pH control, sample extraction/transfer and in-line spectroscopic analysis. Except for spectrometers and light sources, the chemical reaction module together with stock solutions are contained in a temperature-controlled box for the fine tuning of the reaction conditions to ensure reproducibility. The module is equipped with a seed extraction system for sample storage to run new reactions from the previously synthesised nanoparticles. For the discovery of AuNP, the closed loop incorporates three steps including (1) parallel seed-mediated synthesis for a batch of reaction conditions suggested by algorithms that requires liquid dispensing and dynamic pH control, (2) spectroscopic analysis of the products together with cleaning steps to prepare for the next synthesis, and (3) data analysis involving feature extraction to generate new reaction conditions using ML algorithms. The complete iteration cycle, chemical reaction module and overall experimental platform are shown in FIG. 2. Full details of the platform design, construction and operations are described above.

### AI-EDISON: Quality-Diversity Algorithms for Nanomaterials Discovery

The exploration and optimisation strategies to discover different nanostructures. They are based on MAP-Elites and global search with local sparseness algorithms, respectively. In the exploration, AI-EDISON aims to facilitate the diversity in the observation space, which is derived from the UV-Vis spectra of the nanoparticles obtained from various synthetic conditions. Inspired by the MAP-Elites algorithm³⁶, the complete behaviour space is discretized into finite intervals called as classes. Each sampling point corresponding to an experiment is classified, and a pre-defined fitness function is evaluated. The sampling points with the highest fitness in each class are defined as elites, which are then used as the parent set to generate new sampling points via mutation, crossover, and random sampling that are commonly used in evolutionary algorithms. In the context of our exploration, the sampling points represent the synthetic conditions, and the spectral wavelength range (400-950 nm) is discretized into multiple intervals.

To increase the diversity of the UV-Vis spectra, a set of fitness functions are defined to facilitate relative prominences of spectral signals, e.g., to leas to spectra with a single dominant peak or two prominent peaks. By combining the intervals where dominant UV-Vis peaks are located and together with the selected fitness function, the sampling points are classified. Hence, the synthetic conditions are classified into the individual classes based on the extracted peak positions and the selection of fitness functions. As a final step, the fitness functions of the sampling points are evaluated to select the highest-performance sample from each class. The selected samples which form the elite set can be used as the parents to create the new synthetic conditions that will be further evaluated. The emergence of samples with high fitness values in various classes enables the search of synthetic conditions for the preparation of nanoparticles with both diversified and optimal morphologies. This complete process iterates until the exploration is finished.

In the optimisation mode, AI-EDISON searches synthetic conditions to produce samples toward a pre-defined target spectrum. The target spectrum can be the available spectrum from literature, or the simulated spectrum of an estimated nanostructure from electron micrographs. The later strategy uses the structural information from exploration and offers more practical targets. Due to the lack of unique linkage between the morphology and UV-Vis spectrum, multiple nanostructures sharing similar spectral features can be fabricated in the same synthetic space with varied conditions. AI-EDISON searches multiple synthetic conditions by considering the similarity metric that quantifies from the difference between sample and target spectra, together with the local sparseness of sampling points in the synthetic space. The local sparseness indicates the local sampling density and is calculated by estimating the average Euclidean distance between the sampling point and its K-nearest neighbours. To enable the global search, the fitness function for a sampling point is defined by using a linear combination of the similarity metric and local sparseness. The top-N sampling points with the highest fitness are selected as parents and new synthetic conditions are generated via mutation, crossover, and random sampling.

### In Silico Evaluation of the Performance of Exploration and Optimisation Algorithms

The exploration and optimisation algorithms in AI-EDISON were benchmarked in a simulated chemical space with calculated spectral properties. The simulated space contains parameters describing the three-dimensional solid mimicking the nanoparticle shape, metal composition (Au/Ag), and yield, see FIG. 3a. The input chemical space comprises five parameters (v₁, v₂, v₃, v₄, v₅), where (v₁, v₂, v₃) describes the nanoparticle shape using superellipsoid as the shape descriptor, 14 represents relative silver concentration, and v₅ describes the nanoparticle yield in the presence of octahedral Au—Ag bimetallic NPs as by-products. The observation space of UV-Vis spectra was generated the extinction spectrum simulation. The scheme for the exploration to facilitate the UV-Vis diversity and to optimize spectral features is available in FIG. 3b. The exploration algorithm demonstrates a very efficient discovery of high-performance and diverse samples in the simulated chemical space outperforming random search, and the average fitness of the highest-performance samples from different classes eventually reaches 98% to the estimated maximum. The complete flow diagram, interconnected classes of various phase volumes and the algorithm's accelerated performance as compared to Random Search are shown in FIG. 3b-d.

The optimisation strategy towards a target spectrum continued based on the dataset gathered during exploration. Considering the non-uniqueness of the UV-Vis spectra to a specific morphology of nanostructure, the optimisation is set up to find multiple sampling points corresponding to global and local maxima in the similarity landscape (see FIG. 3d). Without setting an explicit target, it is unlikely that the exploration algorithm can accidentally find a solution with similar UV-Vis to the target, as shown in FIGS. 3e and 3f. Thus, a fitness function is crucial to guide the directive optimization. The fitness function (F) is defined by the linear combination of the similarity metric (α) and the local sparseness (β) by F=α+kβ, where the linear coefficient k tunes the weights between enabling similarity to the target and sampling in the less explored region. To evaluate the performance of the optimisation algorithm, the input space was first explored for 11 steps, then a target spectrum was assigned, and optimisation was performed by setting different values of the linear coefficient k. The selection of the optimal k value is crucial for the efficiency of the optimisation. At the lower k values of 0 and 50, the optimisation strategy attempted to find a global maximum with a high probability of getting trapped in a local maximum. At higher values such as k=100, the optimisation algorithm found the global maximum efficiently. However, further increasing k≥200 led to exploration in the sparsely sampled space with a less preference for similarity, causing a decrease in optimisation efficiency. The complete flow diagram, optimisation target, optimisation performance and optimised solutions are shown in FIGS. 3e-h.

### Exploration of Chemical Spaces of Au NPs Using AI-EDISON

With AI-EDISON, three hierarchically linked chemical spaces with potential 1023 experiments were explored with varyied synthetic conditions, where diversified morphological features emerged in the seed-mediated synthesis. A single exploration step has 23 reactions unless explicitly mentioned, each with experimental constraints such as constant total volume, temperature, and synthesis interval. An additional well-defined experiment was performed at each step to verify the stability and precision of the control hardware and characterisation. Because to different observations in UV-Vis spectra in the three chemical spaces, the fitness functions and definition of classes were modified accordingly.

### Chemical Space 1: Seed-Mediated Synthesis on Cuboctahedron Single Crystals

In the first chemical space, exploration toward diverse nanostructures was performed using a single crystal cuboctahedron seed (ca. 2 nm), which was prepared from the fast reduction of HAuCl₄ using NaBH₄ in the presence of hexadecyltrimethylammonium bromide (CTAB)⁴⁷. The parameters for the first chemical space were defined by the volumes of CTAB, AgNO₃, HAuCl₄ and ascorbic acid while keeping the volume of seed solution fixed at 0.5 mL. A total volume of 12 mL was introduced as an additional constraint by adding water if required. At each exploration step, the sampling points were distributed to single or multiple-peak features based on the analysis of their spectra. With peak number, peak positions, and the selection of fitness functions, they were classified into respective classes. Based on the exploration criteria towards a single dominant and two prominent peaks, the set of fitness functions leading to different classes are described in Section 3 above.

Starting with random sampling at the first step, the observed highest-performance samples within various classes with both single-peak and multiple-peak features were used as the parents to generate new sampling points in synthetic space. The samples of classes that did not exist after the initial random sampling, can be generated by mutation and crossover from the parent set as well as random sampling. The exploration ran for 10 steps with a total of 230 experiments. During the exploration, the best samples in the parent set were updated up to 42 times via crossover and mutation operations. Only four events (<10%) were observed, where a new elite with higher-performance or belonging to a previously nonexistent class was generated via crossover or mutation from previous parents with different peak numbers to it, indicating relatively weak interactions between single and multiple peak features. At this stage, electron micrographs obtained from the secondary characterisation of TEM confirmed the presence of Au nanospheres and nanorods. Hence, as an additional phase, we extended the exploration further by orienting towards constrained exploration and exploitation using a new fitness function to improve the performance observed in diversified samples. The new fitness function was selected to primarily increase the contribution of a single peak and lower the secondary peak, where the absorption from the by-products was considered explicitly. Six additional steps were performed with different coefficients in the fitness function, which were used among steps of 11-14 and 15-16. A total of six distinct Au NPs with synthetic conditions leading to high yield and monodispersity were discovered, see FIG. 4 L1-1 to L1-6. The exploration in this chemical space found symmetry-breaking as a key phenomenon towards the emergence of observed nanospheres and nanorods.

### Chemical Space 2: Overgrowth of Au Nanorods

In the second chemical space, the sample of Au nanorods (L1-5) found from the previous chemical space was selected as seed due to its relatively high aspect ratio and presence of the concave features on the surface. Hydroquinone (HQ) was used as the reductant and the pH of the growth solution was introduced as an additional variable. Due to the relatively weak interactions between single and multiple-peak features as observed during the exploration in the first chemical space, these features were explored sequentially. Starting with multiple peak features, the exploration was performed towards a single dominant and two comparable peaks by utilising peak position and relative prominences similarly to previous chemical space. The exploration ran for 10 steps (total 230 reactions) excluding single peak outcomes, and three classes of nanorods were discovered: (a) rods with spherical caps, (b) rods with rectangular caps, and (c) irregular rods resembling dog bones. The total of 10 discovered nanorods of different sizes and belonging to three classes are shown in FIG. 4 L2-1 to L2-10.

To increase further diversity in single peak feature, the exploration of single peak feature ran for 10 steps initialised with the data from the multiple-peak exploration. New single peak classes were defined by discretizing the wavelength of 400-600 nm with 25 nm interval and 600-950 nm with 50 nm interval. Previously, there were four classes found from the initial dataset and the total number of discovered classes increased to 10 at the end of exploration. With the single peak feature, the synthetic conditions for three additional morphologies of (a) spherical polyhedra, (b) bicones and (c) rods with low aspect-ratios were found. The spherical polyhedra and bicones were transformed into highly monodispersed spheres after being aged for 16 hours. See FIG. 4 L2-11 to L2-13 for these nanostructures and the respective UV-Vis spectra. The discovery of various anisotropic and symmetric nanoparticles seeded from smaller nanorods demonstrates the existence of different overgrowth phenomena leading to the emergence of diverse nanostructures.

### Chemical Space 3: Overgrowth of Au Nanospheres

In the third chemical space, the sample of spherical nanoparticles (L2-12-2) was selected as the seed due to their high monodispersity and smooth surface. The five-dimensional input chemical space was defined by volumes of hexadecyltrimethylammonium chloride (CTAC), AgNO₃, HAuCl₄, ascorbic acid and HCl. The volume of seed solution used was fixed to 0.5 mL and the total volume was constrained to 12 mL. The exploration algorithm ran for 10 steps (231 experiments with 24 from the initial random sampling) focusing only on the single peak feature while sampling points leading to multiple peaks were discarded.

The classes were defined by defining the region between 400-550 nm as a single class, as well as discretizing 550-800 nm with 25 nm interval and 800-950 nm with 50 nm interval. The algorithm found 11 high-performance samples of different classes and discovered synthetic conditions leading to a series of nanostars with sizes ranging between 60-95 nm and various tip features, see Figures L3-1 to L3-5 and the corresponding UV-Vis spectra. The morphology of L3-1 comprises a 60 nm core with tiny tips on the surface leading to lower peak absorbance (ca. 560 nm). The peak position redshifts with the increase in core size as evident by absorbance peaks of UV-Vis spectra of nanostars. The algorithmic discovery of the existence of nanostars with variable core sizes and tip features with high yield and monodispersity occurred due to the presence of distinct peak absorbances in the UV-Vis spectra with optimal broadness.

The successful search of a variety of uniquely-shaped Au NPs with high yield and monodispersity using AI-EDISON validates the initial hypothesis that the structural diversity of nanostructures can be achieved by increasing the diversity of the spectra. It also demonstrates that amplifying UV-Vis features like prominence or broadness can improve the yield and monodispersity of the synthesised nanostructures.

### Targeted Optimisation in the Explored Chemical Space

The exploration algorithm discovered synthetic conditions of nanoparticles with distinct UV-Vis behaviours in a coarse-grained way, which can be limited by class intervals without a specific target. To efficiently search synthetic conditions towards finely tuned optical properties utilising the previously explored dataset, the optimisation algorithm with specific targets should be used. Considering the non-uniqueness of UV-Vis spectra towards a single nanostructure, the fitness function is defined by a combination of local sparseness and similarity towards the target spectrum. The target spectrum can be defined either from a literature report or generated in-silico after creating a three-dimensional nanostructure derived from electron micrographs, which offers the more practical targets in the chemical space. To demonstrate efficient optimisation in high-dimensional space with multiple possible solutions, two target spectra were generated in-silico in the first and third chemical space.

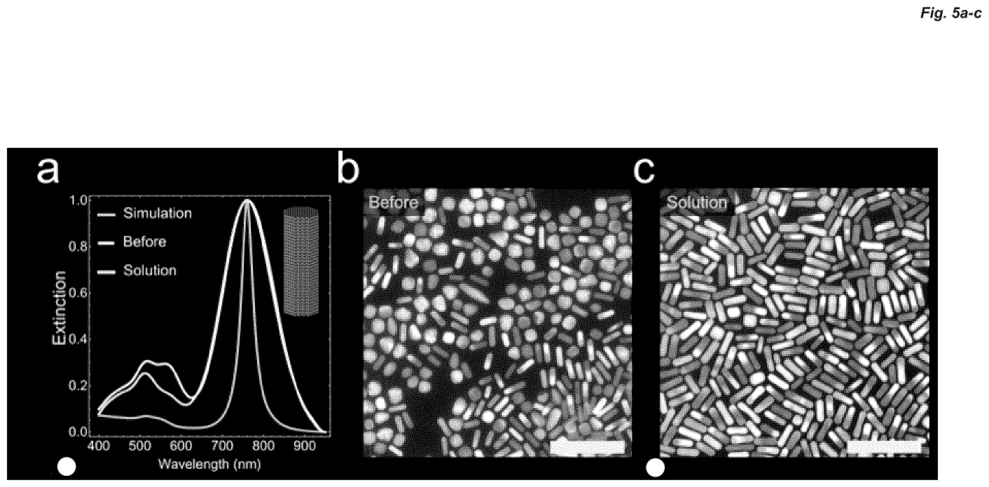

In the first case, the existence of Au nanorods was observed in chemical space 1 during exploration. The target spectrum was simulated using a cylindrical Au nanorod with diameter and length of 11 and 33 nm respectively to precisely control the longitudinal peak. After the exploration, although the sample with the highest similarity shared the same longitudinal peak position, the presence of a shoulder peak around 570 nm indicated the existence of by-products. The optimisation ran for five steps (115 reactions) and two synthetic conditions leading to a smaller by-product peak were obtained (FIG. 44). They showed similar morphological features of nanorods due to the relatively unique transverse and longitudinal UV-Vis peaks (spectra not shown). FIG. 5a shows the UV-Vis spectra of the target, the best solution before optimisation, and one of the solutions with the highest nanorod yield after optimisation. The corresponding electron micrographs are shown in FIGS. 5b-c, indicating the increase of shape yield in the solution from 57% to 95%.

In the second case, during the exploration in the third chemical space, although only a small portion of Au octahedra was observed (spectra not shown), the structural features of the sample as well as L3-1 suggested a high propensity towards the emergence of octahedral nanoparticles. Hence, Au octahedra with edge length ca. 57 nm were created and the target spectrum was simulated. The spectrum has an intrinsically broad peak due to its geometry and is independent of the size distribution. The presence of this intrinsic broadness decreases the uniqueness of UV-Vis towards a specific nanostructure. A variety of nanostructures including spheres, octahedra, other polyhedral shapes of various sizes and their mixtures can share similar UV-Vis features. Hence, it is necessary for the optimisation strategy to search for multiple solutions well-separated in a high dimensional chemical space, which are likely to correspond to different structures. The synthetic space for the optimisation was selected similar to chemical space 3 during exploration except for the concentration of HAuCl₄, which was halved. This reduction was based on the observation that the top five sampling points in the combinatorial space with the highest similarity to the target after the exploration had a small volume of HAuCl₄ (<1.00 mL). The optimisation algorithm ran for 5 steps (115 reactions) and multiple solutions with distinct synthetic conditions but high spectral similarities to the target were found. These synthetic conditions corresponded to local maxima in the fitness landscape and resulted in different nanostructures including octahedral, concave octahedral and smooth polyhedral nanoparticles (see FIGS. 5d-i), which demonstrated successful search of distinct nanostructures with similar UV-Vis spectra to the target through the optimization strategy.

### Multistep Nanoparticle Synthesis Using AI-EDISON Architecture

The modularity of the platform allows conducting parallel multistep synthesis using a generic directed graph structure to easily access any discovered nanoparticles, together with required characterisation at each step to ensure synthesis reproducibility. The abstract synthesis of nanoparticles is represented by a synthesis graph, where each node represents a unique nanoparticle with directed edges representing the hierarchical relationship among various nanoparticles. To map the synthesis graph to the robotic platform, a reaction graph that constitutes the required robotic operations is prepared. Furthermore, a hardware graph is derived from the reaction graph to allocate the available resources of the chemical reaction module. Each node in the reaction and hardware graph represents an actual sample to be prepared, and the directed edges represent the transfer steps required for seeding from one sample to another. The number of generated samples is estimated based on the volume required for seeding, characterisation, and desired final volume.

Six samples of uniquely-shaped AuNPs from the previous exploration of the chemical spaces were chosen and a synthesis graph was generated based on their hierarchical relationship. The parallel synthesis of all six nanoparticles was repeated three times to demonstrate reproducibility. The observed standard deviation in the UV-Vis spectra is 3-4 nm with a maximum deviation around 7 nm. For every nanoparticle, all the peak positions of the repeats are within two standard deviations from their mean values. The unique digital signatures of AuNPs were created from their synthetic procedures and validation of the products through a hash function. The universal chemical programming language χDL⁴⁶, which is independent of hardware, was utilised as the standard way to describe the synthetic procedures, which ensured the reliable synthesis of nanoparticles with expected properties either in any suitable robot or even manually on demand. The validation of the products can reply on various techniques, and UV-Vis was selected for the system of AuNPs due to their plasmonic effect.

The synthetic flow scheme, synthesis/reaction/hardware graphs, original and reproduced UV-Vis spectra of all six Au NPs, parallel synthesis on the chemical reaction module and the procedure to create the digital signatures are shown in FIG. 6. See Section 5 for a complete description of the implementation.

### SUMMARY

The present invention provides a unified architecture AI-EDISON that includes a fully autonomous closed-loop synthesis robot that incorporates state-of-the-art ML algorithms and an extinction spectrum simulation engine. Using quality-diversity algorithms, we explored three linked chemical spaces and discovered AuNPs including spheres, rods, spherical polyhedra, bicones, and stars with diversified features. Although UV-Vis cannot offer detailed structural information of nanoparticles like crystallographic phases or electron density distributions compared to electron microscopy, it was sufficient to target distinct plasmonic nanostructures. By exploration with UV-Vis as a primary characterization tool, we proved our hypothesis that structural diversity can be achieved by increasing the spectral diversity and that demonstrated nanoparticles with high yield and monodispersity can be obtained by amplifying specific spectral features. After coarse-grained exploration of high-dimensional chemical space, the system performed optimisation for finely tuned optical properties with a target spectrum generated using electron microscopy and extinction spectrum simulation. The optimisation strategy discovered multiple synthetic conditions that lead to distinct nanostructures with high yield, monodispersity and similar UV-Vis features, which were not directly available from the exploration. Using the modularity and capacity to perform parallel operations and synthesis on AI-EDISON, we demonstrated a highly efficient and fully digitised approach toward the complex multistep synthesis of nanomaterials with their unique digital signatures derived from χDL.

### REFERENCES

A number of publications are cited above in order to more fully describe and disclose the invention and the state of the art to which the invention pertains. Full citations for these references are provided below. The entirety of each of these references is incorporated herein.
1. Cheng et al. Chem. Rev. 114, 10869-10939 (2014).
2. Radisavljevic et al. Nat. Nanotechnol. 6, 147-150 (2011).
3. Cao et al. Chem. Soc. Rev. 45, 4747-4765 (2016).
4. Hoang et al. Nano Lett. 16, 270-275 (2016).
5. Sosa et al. J. Phys. Chem. B 107, 6269-6275 (2003).
6. Yu et al. Mater. Chem. Phys. 136, 334-340 (2012).
7. Yang et al. ACS Nano 12, 4605-4614 (2018).
8. Shepherd et al. Chem. Commun. 55, 11880-11883 (2019).
9. Scarabelli et al. J. Phys. Chem. Lett. 6, 4270-4279 (2015).
10. Ali et al. Nanotechnol. Sci. Appl. 9, 49-67 (2016).
11. Marlett et al. Ann. N. Y. Acad. Sci. 125, 12-24 (1965).
12. Esumi et al. Langmuir 11, 3285-3287 (1995).
13. Lee et al. Nat. Commun. 11, 1-10 (2020).
14. Nikoobakht et al. Chem. Mater. 15, 1957-1962 (2003).
15. Wu et al. Inorg. Chem. 50, 8106-8111 (2011).
16. Ojea-Jiménez et al. J. Phys. Chem. C 115, 15752-15757 (2011).
17. Mountrichas et al. J. Phys. Chem. C 118, 22754-22759 (2014).
18. Baber et al. Nanoscale 9, 14149-14161 (2017).
19. Lee et al. Nat. Commun. 6, (2015).
20. Steiner et al. Science (80). 363, (2019).
21. Salley et al. ACS Cent. Sci. 6, 1587-1593 (2020).
22. Li et al. Science (80). 347, 1221 LP-1226 (2015).
23. Burger et al. Nature 583, 237-241 (2020).
24. Chang et al. Sci. Rep. 10, 1-9 (2020).
25. Epps et al. Adv. Mater. 32, 1-9 (2020).
26. Langner et al. Adv. Mater. 32, (2020).
27. Tao et al. Adv. Funct. Mater. 2106725, 1-9 (2021).
28. Raccuglia et al. Nature 533, 73-76 (2016).
29. Oliynyk et al. Chem. Mater. 28, 7324-7331 (2016).
30. Salley et al. Nat. Commun. 11, 1-7 (2020).
31. Tao et al. Nat. Rev. Mater. 6, 701-716 (2021).
32. Li et al. Nat. Commun. 11, 1-10 (2020).
33. Kusne et al Nat. Commun. 11, 1-11 (2020).
34. Pugh et al. Front. Robot. AI 3, 1-17 (2016).
35. Lehman et al. Proc. 13th Genet. Evol. Comp. Conf 211-218 (2011).
36. Mouret, J.-B. & Clune, J. Illuminating search spaces by mapping elites. 1-15 (2015). 36.
37. Arulkumaran et al. GECCO 2019 Companion—Proc. 2019 Genet. Evol. Comput. Conf. Companion 314-315 (2019) doi: 10.1145/3319619.3321894.
38. Kaushik et al. Front. Robot. AI 6, (2020).
39. Verhellen et al Chem. Sci. 11, 11485-11491 (2020).
40. Grizou et al. Sci. Adv. 6, (2020).
41. Rao, A. et al. J. Phys. Conf. Ser. 61, 971-976 (2007).
42. Vladár et al. Characterization of Nanoparticles: Measurement Processes for Nanoparticles (Elsevier Inc., 2019). doi: 10.1016/B978-0-12-814182-3.00002-X.
43. Wang et al. J. Phys. Chem. B 104, 1153-1175 (2000).
44. Zheng et al. ACS Appl. Mater. Interfaces 8, 21585-21594 (2016).
45. Li et al. Chem. Rev. 116, 11128-11180 (2016).
46. Hessam et al. Science (80). 370, 101-108 (2020).
47. Walsh et al. ACS Nano 9, 715-724 (2015).

### References from Sections 1.1. to 5.3

1. Salley et al. ACS Cent. Sci. 6, 1587-1593 (2020).
2. Mouret, J.-B. & Clune, J. Illuminating search spaces by mapping elites. 1-15 (2015).
3. Lehman et al. Proc. 13th Genet. Evol. Comp. Conf 211-218 (2011).
4. Gui et al. J. Nanomater. 2015, (2015).
5. Marcheselli et al. J. Chem. Theory Comput. 16, 3807-3815 (2020).
6. Draine et al. J. Opt. Soc. Am. A 11, 1491 (1994).
7. Piller et al. IEEE Trans. Antennas Propag. 46, 1126-1137 (1998).
8. Concerning, R. & Law, T. H. E. C. Remarks concerning the clausius-mossotti law. 49, (1929).
9. Wang et al. Sci. Adv. 3, 1-9 (2017).
10. Thambi et al. ACS Omega 4, 13733-13739 (2019).
11. Hessam et al. Science (80) 370, 101-108 (2020).

### Other References

U.S. Pat. No. 5,463,564
WO 2009/053691
WO 2013/175240

1. A method for the exploration of chemical space, the method comprising a multigenerational series of synthetic stages, including:
(i) a first stage, where a first series of reactions is performed, and an analysis of the reaction product for each reaction in the first series is performed, and a first product from the first series of reactions is selected; and
(ii) a second stage, where a second series of reactions is performed, where the selected product from the first stage is provided as a chemical input to each of the reactions in the second series, and an analysis of the reaction product for each reaction in the second series is performed, and a product from the second series of reactions is optionally selected,
wherein the first and second stages are performed autonomously, and the selection of a product in the first stage comprises the comparison of the products from the first series of reactions against a fitness function, where the selected product has a superior fitness compared with one or more other products in the first series, and
each reaction in the first series differs in one or more chemical and/or physical inputs, and each reaction in the second series differs in one or more chemical and/or physical inputs.

2. The method of claim 1, wherein:
(i) in the first stage, a first series of reactions is performed, and an analysis of the reaction product for each reaction in the first series is performed, and a first and second product from the first series of reactions is selected; and
(ii) in the second stage, a second series of reactions is performed, where the first selected product from the first stage is provided as a chemical input to each of the reactions in the second series, and an analysis of the reaction product for each reaction in the second series is performed, and a product from the second series of reactions is optionally selected, and
the method further comprising:
(iii) a third stage, where a third series of reactions is performed, where the second selected product from the first stage is provided as a chemical input to each of the reactions in the third series, and an analysis of the reaction product for each reaction in the third series is performed, and a product from the third series of reactions is optionally selected,
wherein the first, second and third stages are performed autonomously, and the selection of the first and second products in the first stage comprises the comparison of the products from the first series of reactions against a fitness function, where the selected products have a superior fitness compared with one or more other products in the first series, and
each reaction in the first series differs in one or more chemical and/or physical inputs, each reaction in the second series differs in one or more chemical and/or physical inputs, and each reaction in the third series differs in one or more chemical and/or physical inputs.

3. The method of claim 1, wherein the method further comprises:
(iv) an additional optimisation stage, where a series of reactions is performed, where the selected product from an earlier stage, such as a selected product from the second stage, or a selected product from a third stage, where present, is provided as a chemical input to each of the reactions in the additional series, and an analysis of the reaction product for each reaction in the additional series is performed, and a product from the additional series of reactions is optionally selected,
wherein the additional stage is performed autonomously, and the selection of a product in the additional stage comprises the comparison of the products from the additional series of reactions against a fitness function, where a selected product has a superior fitness compared with one or more other products in the first series, and
each reaction in the additional series differs in one or more chemical and/or physical inputs.

4. The method according to claim 3, wherein the method comprises two or more, such as three or more, additional optimisation stages in generation, wherein each subsequent additional optimisation stage uses the selected product from the previous optimisation stage.

5. The method according to claim 1, wherein the method proceeds continuously within each stage.

6. The method according to claim 1, wherein the method proceeds continuously from one stage to a following stage.

7. The method according to claim 1, wherein a part of a later series of reactions may be performed coincident with the performance of a part of an earlier series of reactions.

8. The method according to claim 1, wherein the fitness function is determined against a spectroscopic property of each product.

9. The method according to claim 8, wherein the spectroscopic property is measured by UV-vis or IR spectroscopy.

10. The method according to claim 1, wherein a selected product is an elite product.

11. The method according to claim 1, wherein the method is for the preparation of products that are particles, rods or fibres, such as nanoproducts that are nanoparticles, nanorods or nanofibres.

12. The method according to claim 1, wherein the first stage of the method uses a starter reagent as a chemical input to each of the reactions in the first series.

13. The method according to claim 2, wherein the selection of first and second products from the first reaction stage comprises selecting first and second products having structural or chemical dissimilarity.

14. An automated exploration apparatus for performing a method for the exploration of chemical space, and the apparatus comprises a controller, and a chemical synthesiser and an analytical unit which are operable by the controller, wherein:
(i) the chemical synthesiser having a plurality of reaction spaces; a supply of chemical inputs, optionally together with physical inputs for supplying to the chemical inputs, optionally together with physical inputs to a reaction space; a product separator for at least partial separation of a reaction product from a reaction product mixture in a reaction space, and for supply of the reaction product to a separate reaction space;
(ii) the analytical unit for analysing a product of a reaction performed in a reaction space of a chemical synthesiser; and
(iii) the controller is suitably programmed to operate the chemical synthesiser and the analytical unit; to receive analytical information from the analytical unit; and to compare the analytical information for reaction products against a fitness function; to make a selection of a reaction product for the product separator to supply to a separate reaction space; and to make a selection of chemical inputs, optionally together with physical inputs, to the reaction spaces
and the controller is adapted to operate the chemical synthesiser and the analytical unit autonomously.

15. The automated exploration apparatus according to claim 14, wherein the plurality of reaction spaces is the plurality of reaction spaces with a Geneva wheel.

16. The automated exploration apparatus according to claim 14, wherein the analytical unit comprises a UV-vis spectrometer or an IR spectrometer.

17. The automated exploration apparatus according to claim 14, wherein the controller is suitably programmed with an AI algorithm to analyse analytical data and to select products for use in a series of reactions, and to select future chemical inputs, optionally together with physical inputs, for use with the selected product a series of reactions.

18. A library which is a collection of a plurality of products obtained or obtainable from the methods of claim 1, such as the collection of products obtained or obtained from each series of reactions.

19. The library of claim 18, wherein the plurality of products is a collection of the selected products from each stage in the method.
